(19) United States
(12) Patent Application Publication
(10) Pub. No.: US 2020/0362368 A1
(43) Pub. Date: Nov. 19, 2020

(54) METHODS OF REDOSING GENE THERAPY VECTORS

(71) Applicant: BIOMARIN PHARMACEUTICAL INC., Novato, CA (US)

(72) Inventors: Peter COLOSI, Fairfax, CA (US); Justin ISHIDA, San Rafael, CA (US); Silvia RAMIREZ, Windsor, CA (US)

(73) Assignee: BIOMARIN PHARMACEUTICAL INC., Novato, CA (US)

(21) Appl. No.: 15/931,180

(22) Filed: May 13, 2020

Related U.S. Application Data
(60) Provisional application No. 62/847,908, filed on May 14, 2019.

(51) Int. Cl. C12N 15/86; A61P 7/00

(52) U.S. Cl. CPC C12N 15/86; A61P 7/00; C12N 2750/14121; C12N 2750/14122; A61K 45/06; C12N 2750/14143; C12N 2830/008; C12N 2830/48; C12N 2830/85; C12N 2750/14142

(57) ABSTRACT

The present disclosure relates, in general, to methods for readministering, or redosing, a subject having undergone a first gene therapy regimen with a second, or subsequent, administration of a gene therapy regimen, wherein the first gene therapy vector and second gene therapy vector comprise different AAV capsids but carry a transgene or polynucleotide useful to treat the same disease or disorder.

### CROSS-REFERENCE TO RELATED APPLICATIONS

This application claims the benefit of U.S. Provisional Application No. 62/847,908, filed May 14, 2019, which is incorporated by reference herein in its entirety.

### REFERENCE TO SEQUENCE LISTING SUBMITTED ELECTRONICALLY

This application incorporates by reference a Computer Readable Form (CRF) of a Sequence Listing in ASCII text format submitted with this application, entitled “11808-459-999_SEQ_LISTING.txt”, was created on May 11, 2020, and is 1,065,489 bytes in size.

### FIELD OF DISCLOSURE

The present disclosure relates to methods for re-administration or redosing of gene therapy vectors, for example AAV vectors, in order to avoid the immune response to the initial vector, or to evade the immune response to the first AAV vector and enable therapeutic expression of a subsequent dose of AAV vector.

### BACKGROUND

Adeno-associated virus (AAV) is considered as one of the most promising viral vectors for human gene therapy. AAV has the ability to efficiently infect dividing as well as non-dividing human cells. The wild-type AAV viral genome integrates into a single chromosomal site in the host cell's genome, and most importantly, even though AAV is present in many humans it has not been associated with any disease. In view of these advantages, recombinant adeno-associated virus (rAAV) is being evaluated in numerous gene therapy clinical trials for a variety of different clinical indications.

Adeno-associated virus (AAV), a member of the Parvovirus family, is a small replication-deficient, nonenveloped, icosahedral virus with single-stranded linear DNA genomes of 4.7 kilobases (kb) to 6 kb. The nucleotide sequence of the AAV serotype 2 (AAV2) genome is presented in Srivastava et al., J. Virol., 45: 555-564 (1983) as corrected by Ruffing et al., J. Gen. Virol., 75: 3385-3392 (1994). The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy and expressing therapeutic peptides/polypeptides and polynucleotides. AAV infection of cells in culture is non-cytopathic, and natural infection of humans and other animals is silent and asymptomatic. Moreover, AAV infects many mammalian cells allowing the possibility of targeting many different tissues in vivo. The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of rAAV-vectors less critical. AAV may even be lyophilized. Finally, AAV-infected cells are not resistant to superinfection.

One major challenge for a successful administration of AAV vector is to overcome the presence of neutralizing antibodies (immunoglobulins) (NAb) that have developed following exposure to wild-type AAV or AAV-based vectors. In both cases, the neutralizing serotype-specific antibodies directed towards the viral capsid proteins can reduce the efficiency of gene transfer with AAV of the same serotype. (Scallon et al., Blood. 107:1810-7, 2006; Hurlbut et al., Mol Ther. 18:1983-1994, 2010) AAV is commonly found in the environment and natural, pre-existing immunity such as neutralizing antibodies to many known AAV exists in the majority of the human population, including children (Fu et al., Hum Gene Ther Clin Dev. 28:187-196, 2017). Hence, the general consensus in the field is to avoid treating patients having NAb titers altogether. Thus, the current practice in the clinic with regard to pre-existing immunity involves the screening of human patients for exclusion should patients have neutralizing antibodies against the AAV capsid (Brimble et al. Expert Opin Biol Ther 2016, 16(1):79-92 and Boutin et al. Hum Gene Ther 2010, 21:704-712). Immunosuppressive regimens have been tried in order to reduce the formation of NAb upon first administration to allow for a second administration (Corti et al., Mol Ther-Meth Clin Dev (2014) 1, 14033; Mingozzi et al. Mol Ther vol. 20 no. 7, 1410-1416; McIntosh et al. Gene Ther 2012, 19, 78-85)). Furthermore, strategies have been suggested to overcome pre-existing antibodies which include plasma exchange and the use of immunosuppressive regimens (e.g. Chicoine et al., Mol Ther 2014, vol. 22 no. 2 338-347; Hurlbut et al. Mol Ther 2010, vol. 18 no. 11 1983-1984 and Mingozzi et al. Mol Ther vol. 20 no. 7, 1410-1416). These strategies have been tested in animal models obtaining limited success.

Another challenge for the AAV vector gene transfer platform is the durability of the effect. Despite the exciting results to date in human clinical gene therapy trials with AAV demonstrating durable expression at therapeutic levels when targeting tissues like the liver (George et al., N. Engl. J. Med. 377, 2215-2227 (2017); Manno et al., Nat. Med. 12, 342-347 (2006); Miesbach et al., Blood 131, 1022-1031 (2018); Nathwani et al., N. Engl. J. Med. 371, 1994-2004 (2014); Nathwani et al., N. Engl. J. Med. 365, 2357-2365 (2011); Rangarajan et al., N. Engl. J. Med. 377, 2519-2530 (2017)), motor neurons (Mendell et al., N. Engl. J. Med. 377, 1713-1722 (2017)), and the retina (Russell et al., Lancet 390, 849-860 (2017)), for many metabolic and degenerative diseases, treatment is critically needed early in life (Fagiuoli et al., J. Hepatol. 59, 595-612 (2013); Soriano et al., J. Pediatr. Gastroenterol. Nutr. 35(Suppl 1), S51-S54 (2002)), prior to the onset of irreversible tissue damage. However, because of their non-integrative nature, systemic gene therapy with AAV vectors in pediatric patients is expected to be limited by tissue proliferation associated with organ growth, which results in significant vector dilution over time (Bortolussi et al., Hum. Gene Ther. 25, 844-855 (2014); Ronzitti et al., Mol. Ther. Methods Clin. Dev. 3, 16049 (2016); Wang et al., Hum. Gene Ther. 23, 533-539 (2012)). Thus, maintaining the possibility to re-administer AAV is an important goal to achieve sustained therapeutic efficacy over time in pediatric patients. In addition, vector readministration in both pediatric and adult patients would be desirable to enable vector titration, to increase the proportion of patients that achieve therapeutic levels of the transgene expression, while avoiding supra-physiological transgene expression and potential toxicities associated with large vector doses (Hinderer et al., Hum. Gene Ther. 29, 285-298 (2018)).

However, vector immunogenicity represents a major limitation to re-administration of AAV vectors (Mingozzi et al., Annu Rev. Virol. 4, 511-534 (2017)). Persistent high-titer neutralizing antibodies (NAbs) are triggered following vector administration (Nathwani et al., N. Engl. J. Med. 371, 1994-2004 (2014)), which abolishes any benefit of repeated AAV-based treatments. In addition, experience in human trials has shown that induction of capsid-specific CD8+ T cell responses can lead to clearance of AAV vector-transduced cells (Manno et al., Nat. Med. 12, 342-347 (2006); Nathwani et al., N. Engl. J. Med. 371, 1994-2004 (2014); Nathwani et al., N. Engl. J. Med. 365, 2357-2365 (2011); Mingozzi et al., Nat. Med. 13, 419-422 (2007)).

Thus, there is a need in the art to enable the administration of rAAV gene therapy vectors in human patients that have, or may be suspected to have, AAV neutralizing antibodies. Furthermore, safe and effective strategies aimed at reducing AAV vector immunogenicity that allow for stable transgene expression and vector re-dosing are urgently needed.

### SUMMARY OF THE DISCLOSURE

The disclosure provides for methods of readministering or redosing subjects with a gene therapy vector, wherein the vector for the second or subsequent dosing event comprises a different AAV capsid than the first gene therapy AAV dosing regimen. It is hypothesized that use of first and second AAV vectors that have low capsid sequence homology to each other, and are phylogenetically distinct, will not be effected by the humoral immune response to the first AAV vector compared to readministration of the same gene therapy vector, thereby permitting better transduction efficiency and transgene expression in the subject.

Provided herein is a method of treating a subject with multiple doses of a recombinant adeno-associated virus (rAAV) vector, the method comprising administering to a subject a first rAAV vector comprising a transgene comprising a therapeutic molecule and a first capsid, and administering to a subject a second rAAV vector comprising a transgene comprising a therapeutic molecule and a second capsid, wherein the transgene in the second capsid comprises the same therapeutic molecule or a different therapeutic molecule.

In a specific embodiment, provided herein is a method of treating a subject with multiple doses of an rAAV vector, the method comprising: administering to a subject a first rAAV vector comprising a transgene comprising a therapeutic molecule and a first capsid, and administering to a subject a second rAAV vector comprising a transgene comprising a therapeutic molecule and a second capsid, wherein the transgene in the second rAAV vector comprises the same therapeutic molecule or a different therapeutic molecule as transgene in the first rAAV vector.

Also contemplated herein is a method of treating a disease or disorder in a subject in need thereof with multiple doses of a recombinant adeno-associated virus (rAAV) vector, the method comprising: administering to a subject a first rAAV vector comprising a transgene comprising a therapeutic molecule useful for treating the disease or disorder and a first capsid, and administering to a subject a second rAAV vector comprising a transgene comprising a therapeutic molecule useful for treating the disease or disorder and a second capsid, wherein the transgene in the second capsid comprises the same therapeutic molecule or a different therapeutic molecule useful to treat the disease or disorder.

In a specific embodiment, provided herein is a method of treating a disease or disorder in a subject in need thereof with multiple doses of an rAAV vector, the method comprising: administering to a subject a first rAAV vector comprising a transgene comprising a therapeutic molecule useful for treating the disease or disorder and a first capsid, and administering to a subject a second rAAV vector comprising a transgene comprising a therapeutic molecule useful for treating the disease or disorder and a second capsid, wherein the transgene in the second rAAV vector comprises the same therapeutic molecule or a different therapeutic molecule useful to treat the disease or disorder as the transgene in the first rAAV vector.

In various embodiments, the therapeutic molecule is a therapeutic protein, an inhibitory RNA, an mRNA, or a CRISPR/Cas guide polynucleotide. It is contemplated that the therapeutic molecule in the first capsid can be selected from a therapeutic protein, an inhibitory RNA, an mRNA, or a CRISPR/Cas guide polynucleotide. It is further contemplated that the therapeutic molecule in the second capsid is selected from a therapeutic protein, an inhibitory RNA, an mRNA, or a CRISPR/Cas guide polynucleotide.

In various embodiments, the first and second capsids are phylogenetically distinct. In various embodiments, the phylogenetic difference is based on a threshold level of sequence homology. In various embodiments, the threshold level of sequence homology is approximately less than or equal to 90% sequence homology over the capsid amino acid sequence, or over any one of the VP1, VP2 or VP3 capsid proteins. In various embodiments, the first and second capsids have amino acid sequence homology that is less than or equal to about 90%. In various embodiments, the first and second capsids have less than or equal to about 90% homology in a VP1 capsid protein, have less than or equal to about 90% homology in a VP2 capsid protein and/or less than or equal to about 90% homology in a VP3 capsid protein. In various embodiments, the sequence homology of the capsids, or capsid proteins, may be less than or equal to 90%, 89%, 88%, 87%, 86%, 85%, 84%, 83%, 82%, 81%, 80%, 79%, 78%, 77%, 76%, 75% or lower. In various embodiments, the sequence homology of the capsids, or capsid proteins, may be from about 30% to 90% homologous, from about 45% to 87% homologous, from about 40% to 86% homologous, from about 50% to 85% homologous, or from about 60% to 80% homologous, or from about 65% to 75% homologous.

In various embodiments, the first capsid and/or second capsid exhibit low pre-existing immunity in the subject. In a specific embodiment, a subject exhibits low pre-existing immunity to either the first capsid, the second capsid or both the first and second capsids. In some embodiments, an in vitro assay to measure neutralizing antibody to AAV capsid is used to determine if a subject exhibits pre-existing immunity to the first capsid, the second capsid, or both the first and second capsids

In various embodiments, the subject is human. In various embodiments, the subject is human and is immunologically naïve to the first and/or second AAV vector. In various embodiments, the subject has less than 1:2, 1:5, 1:10, 1:20, 1:50, 1:100, 1:200, 1:300, 1:400, 1:500 or 1:1000 neutralizing antibody to the first or second AAV vector in serum. In certain embodiments, the subject has less than 1:2, 1:5, or 1:10 anti-first AAV vector neutralizing antibody titer or less than 1:100 total anti-first AAV vector-IgG titer in a biological sample (e.g., blood, sera or plasma) from the subject. In certain embodiments, the subject has less than 1:2, 1:5, or 1:10 anti-second AAV vector neutralizing antibody titer or less than 1:100 total anti-second AAV vector-IgG titer. In some embodiments, the subject has less than 1:10, 1:20, 1:50, 1:80, 1:100, 1:200, 1:300, 1:400, or 1:500 total anti-first AAV vector-IgG titer in a biological sample (e.g., blood, sera or plasma) from the subject. In ceratin embodiments, the subject has less than 1:10 anti-second AAV vector neutralizing antibody titer or less than 1:10, 1:20, 1:50, 1:80, 1:100, 1:200, 1:300, 1:400, or 1:500 total anti-second AAV vector-IgG titer. In various embodiments, the neutralizing antibody levels are measured in a neutralizing antibody assay.

In various embodiments, the first capsid is selected from the group consisting of AAV1, AAV2, AAV3, AAV4, AAV5, AAV6, AAV7, AAV8, AAV9, AAV10, AAV11, AAV12, AAV13, AAV3b, LK03, rh74.j, rh10, AAVbo (also referred to herein as “bovine”), AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, Table 6 and/or a variant thereof. In various embodiments, the second capsid is selected from the group consisting of AAV1, AAV2, AAV3, AAV4, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, Table 6, and/or a variant thereof, wherein there is sufficient phylogenetic distance between the viruses that there is not significant cross-reactivity of any preexisting immunogenicity against the first capsid protein toward the second capsid protein.

In some embodiments, anti-first capsid protein antibody(ies) present in a biological sample (e.g., blood, sera or plasma) from a subject does not bind to the second capsid protein as assessed by techniques known in the art, e.g., ELISA, Western blot, biolayer interferometry, FACS or BIACore, or described herein. In certain embodiments, anti-first capsid antibody(ies) present in a biological sample (e.g., blood, sera or plasma) from a subject has a 5 fold, 10 fold, 15 fold, 20 fold, 25 fold or greater fold affinity for the first capsid protein than the second capsid protein as assessed by techniques known in the art, e.g., ELISA, Western blot, biolayer interferometry, FACS or BIACore, or described herein. In some embodiments, anti-second capsid protein antibody(ies) present in a biological sample (e.g., blood, sera or plasma) from a subject does not bind to the first capsid protein as assessed by techniques known in the art, e.g., ELISA, Western blot, biolayer interferometry, FACS or BIACore, or described herein. In certain embodiments, anti-second capsid antibody(ies) present in a biological sample (e.g., blood, sera or plasma) from a subject has a 5 fold, 10 fold, 15 fold, 20 fold, 25 fold or greater fold affinity for the second capsid protein than the first capsid protein as assessed by techniques known in the art, e.g., ELISA, Western blot, biolayer interferometry, FACS or BIACore, or described herein.

In some embodiments, anti-first rAAV vector antibody(ies) present in a sample (e.g., blood, sera or plasma) from a subject does not bind to the second rAAV vector as assessed by techniques known in the art, e.g., ELISA, Western blot, biolayer interferometry, flow cytometry or BIACore, or described herein. In certain embodiments, anti-first rAAV vector antibody(ies) present in a sample (e.g., blood, sera or plasma) from a subject has a 5-fold, 10-fold, 15-fold, 20-fold, 25-fold or greater-fold affinity for the first rAAV vector than the second rAAV vector as assessed by techniques known in the art, e.g., ELISA, Western blot, biolayer interferometry, flow cytometry or BIACore.

In some embodiments, anti-second rAAV vector antibody(ies) present in a sample (e.g., blood, sera or plasma) from a subject does not bind to the first rAAV vector as assessed by techniques known in the art, e.g., ELISA, Western blot, biolayer interferometry, flow cytometry or BIACore, or described herein. In certain embodiments, anti-second rAAV vector antibody(ies) present in a sample (e.g., blood, sera or plasma) from a subject has a 5-fold, 10-fold, 15-fold, 20-fold, 25-fold or greater-fold affinity for the second rAAV vector than the first rAAV vector as assessed by techniques known in the art, e.g., ELISA, Western blot, biolayer interferometry, flow cytometry or BIACore.

In various embodiments, the first capsid and/or second capsid is an engineered or chimeric capsid.

In various embodiments, the first or second capsid comprises a chimeric capsid protein having a VP1 amino acid sequence of a recipient backbone AAV capsid comprising variable regions I, II, III, IV, V, VI, VII, VIII and IX, except wherein one or more of variable regions I, II, III, IV, V, VI, VII, VIII, or IX is replaced by the corresponding variable region from one or more donor AAV capsids. In various embodiments, the one or more variable regions from the recipient AAV capsid is replaced by the corresponding variable region from the donor AAV capsid. In various embodiments, the recipient AAV capsid sequence is any one of SEQ ID NOS:1-89 or 158-164 and the donor AAV capsid sequences are selected from the group of sequences consisting of SEQ ID NOS:1-89 or 158-164, and the recipient AAV capsid sequence and the donor AAV capsid sequences are different. In various embodiments, the chimeric capsid protein comprises the amino acid sequence of any one of SEQ ID NOS:90-157 (see, e.g., Table 7, below).

In various embodiments, the first or second capsid comprises an amino acid sequence that is at least 95% identical to (i) any one of SEQ ID NOs:15-89 or 158-164, (ii) the VP2 region of any one of SEQ ID NOs: 15-89 or 158-164, or (iii) the VP3 region of any one of SEQ ID NOs: 15-89 or 158-164. In various embodiments, the first or second capsid protein comprises the amino acid sequence of (i) any one of SEQ ID NOS:15-89, (ii) the VP2 region of any one of SEQ ID NOS:15-89, or (iii) the VP3 region of any one of SEQ ID NOS:15-89.

In various embodiments, the first capsid protein comprises an amino acid sequence that is at least 95% identical to (i) any one of SEQ ID NOS:158-164, (ii) the VP2 region of any one of SEQ ID NOS: 158-164, or (iii) the VP3 region of any one of SEQ ID NOS: 158-164. In various embodiments, the first capsid protein comprises an amino acid sequence of (i) any one of SEQ ID NOS:158-164, (ii) the VP2 region of any one of SEQ ID NOS: 158-164, or (iii) the VP3 region of any one of SEQ ID NOS: 158-164.

In various embodiments, the second capsid protein comprises an amino acid sequence that is at least 95% identical to (i) any one of SEQ ID NOS:158-164, (ii) the VP2 region of any one of SEQ ID NOS: 158-164, or (iii) the VP3 region of any one of SEQ ID NOS: 158-164. In various embodiments, the second capsid protein comprises an amino acid sequence of (i) any one of SEQ ID NOS:158-164, (ii) the VP2 region of any one of SEQ ID NOS: 158-164, or (iii) the VP3 region of any one of SEQ ID NOS: 158-164.

In various embodiments, the first and second capsid proteins comprise an amino acid sequence that is at least 95% identical to (i) any one of SEQ ID NOS:158-164, (ii) the VP2 region of any one of SEQ ID NOS: 158-164, or (iii) the VP3 region of any one of SEQ ID NOS: 158-164. In various embodiments, the first and second capsid proteins comprise an amino acid sequence of (i) any one of SEQ ID NOS:158-164, (ii) the VP2 region of any one of SEQ ID NOS: 158-164, or (iii) the VP3 region of any one of SEQ ID NOS: 158-164.

In various embodiments, the vector comprises a nucleic acid sequence encoding an adeno-associated virus (AAV) capsid protein having an amino acid sequence that is at least 95% identical to (i) any one of SEQ ID NOS:15-89 or 158-164, (ii) the VP2 region of any one of SEQ ID NOS:15-89 or 158-164, or (iii) the VP3 region of any one of SEQ ID NOS:15-89 or 158-164. In various embodiments, the vector comprises a nucleic acid sequence encoding an adeno-associated virus (AAV) capsid protein having an amino acid sequence of (i) any one of SEQ ID NOS:15-89 or 158-164, (ii) the VP2 region of any one of SEQ ID NOS:15-89 or 158-164, or (iii) the VP3 region of any one of SEQ ID NOS:15-89 or 158-164. In some embodiments, the nucleic acid sequence is operably linked to a heterologous regulatory element that controls expression of the capsid protein in a host cell.

In various embodiments, the host cell is a liver cell or muscle cell.

In various embodiments, the first capsid and/or the second capsid is a muscle-targeting capsid. In particular embodiments, the muscle-targeting capsid is selected from the group consisting of Anc110_9 VR, Bba.26, Bba.41, Bba.42, Bba.43, and Bba.44. In specific embodiments, the muscle-targeting capsid is Bba.41. In various embodiments, the muscle-targeting capsid protein comprises an amino acid sequence that is at least 95% identical to (i) any one of SEQ ID NOS:16, 28, 29, 30, or 31, (ii) the VP2 region of any one of SEQ ID NOS:16, 28, 29, 30, or 31, or (iii) the VP3 region of any one of SEQ ID NOS:16, 28, 29, 30, or 31. In various embodiments, the muscle-targeting capsid comprises an amino acid sequence of (i) any one of SEQ ID NOS:16, 28, 29, 30, or 31, (ii) the VP2 region of any one of SEQ ID NOS:16, 28, 29, 30, or 31, or (iii) the VP3 region of any one of SEQ ID NOS:16, 28, 29, 30, or 31. In specific embodiments, the muscle-targeting capsid protein comprises an amino acid sequence of (i) SEQ ID NOS:28, (ii) the VP2 region of SEQ ID NO:28, or (iii) the VP3 region of SEQ ID NO:28.

In various embodiments, the first capsid and/or the second capsid is a liver-targeting capsid. In particular embodiments, the liver-targeting capsid is selected from the group consisting of Bba.45, Bba.46, Bba.47, Bba.48, Bba.49, Bba.50 and Bba.51. In specific embodiments, the liver-targeting capsid is Bba.47. In specific embodiments, the liver-targeting capsid is Bba.49. In various embodiments, the liver-targeting capsid protein comprises an amino acid sequence that is at least 95% identical to (i) any one of SEQ ID NOS:158-164, (ii) the VP2 region of any one of SEQ ID NOS:158-164, or (iii) the VP3 region of any one of SEQ ID NOS:158-164. In various embodiments, the liver-targeting capsid comprises an amino acid sequence of (i) any one of SEQ ID NOS:158-164, (ii) the VP2 region of any one of SEQ ID NOS:158-164, or (iii) the VP3 region of any one of SEQ ID NOS:158-164. In specific embodiments, the liver-targeting capsid protein comprises an amino acid sequence of (i) SEQ ID NOS:160, (ii) the VP2 region of SEQ ID NO:160, or (iii) the VP3 region of SEQ ID NO:160. In specific embodiments, the liver-targeting capsid protein comprises an amino acid sequence of (i) SEQ ID NOS:162, (ii) the VP2 region of SEQ ID NO:162, or (iii) the VP3 region of SEQ ID NO:162.

In various embodiments, the first or second capsid is selected from the group consisting of AAV5, Bba.49, Bba.47 and bovine. In various embodiments, the first capsid is selected from the group consisting of AAV5, Bba.49, Bba.47 and bovine and the second capsid is selected from the group consisting of AAV5, Bba.49, Bba.47 and bovine. In various embodiments, the first or second capsid is selected from the group consisting of LK03, AAV5, Bba.49 and bovine. In various embodiments, the first capsid is selected from the group consisting of LK03, AAV5, Bba.49 and bovine, and the second capsid is selected from the group consisting of LK03, AAV5, Bba.49 and bovine. In various embodiments, the first capsid or second capsid is selected from the group consisting of rh10, AAV5, Bba.49 and bovine. In various embodiments, the first capsid is selected from the group consisting of Rh10, AAV5, Bba.49 and bovine, and the second capsid is selected from the group consisting of rh10, AAV5, Bba.49 and bovine. In various embodiments, the first or second capsid is selected from the group consisting of AAV8, AAV5, Bba.49 and bovine. In various embodiments, the first capsid is selected from the group consisting of AAV8, AAV5, Bba.49 and bovine, and the second capsid is selected from the group consisting of AAV8, AAV5, Bba.49 and bovine. In various embodiments, the first capsid or second capsid is selected from the group consisting of AAV9 and Bba.41. In various embodiments, the first capsid is selected from the group consisting of AAV9 and Bba.41 and the second capsid is selected from the group consisting of AAV9 and Bba.41.

In various embodiments, the heterologous protein expressed by the transgene in the subject is maintained at a therapeutically effective level.

In various embodiments, the heterologous protein is selected from the group consisting of Factor VIII, Factor IX, ATP7B protein, C1 esterase inhibitor (C1-INH), alpha 1 antitrypsin, and galactose-1-phosphate uridyl transferase (GALT), dystrophin, a mini-dystrophin, microdystrophin, phenylalanine hydroxylase (PAH), alpha-galactosidase A, and glucocerebrosidase.

In various embodiments, expression of the heterologous protein is sufficient to treat a disorder or disease selected from the group consisting of hemophilia A, hemophilia B, Wilson's disease, hereditary angioedema (HAE), alpha 1 antitrypsin deficiency, galactosemia, Duchenne's Muscular Dystrophy or other muscular dystrophies, phenylketonuria (PKU), Fabry Disease, and Gaucher Disease. In various embodiments, the method involves delivering a transgene to a muscle cell or liver cell.

In various embodiments, the method utilizes a first capsid and/or a second capsid that is a muscle-targeting capsid. In particular embodiments, the method utilize a muscle-targeting capsid that is selected from the group consisting of Anc110_9 VR, Bba.26, Bba.41, Bba.42, Bba.43, and Bba.44. In specific embodiments, the method utilizes a Bba.41 muscle-targeting capsid. In various embodiments, the method utilizes a muscle-targeting capsid protein that comprises an amino acid sequence that is at least 95% identical to (i) any one of SEQ ID NOS:16, 28, 29, 30, or 31, (ii) the VP2 region of any one of SEQ ID NOS:16, 28, 29, 30, or 31, or (iii) the VP3 region of any one of SEQ ID NOS:16, 28, 29, 30, or 31. In various embodiments, the method utilizes a muscle-targeting capsid that comprises an amino acid sequence of (i) any one of SEQ ID NOS:16, 28, 29, 30, or 31, (ii) the VP2 region of any one of SEQ ID NOS:16, 28, 29, 30, or 31, or (iii) the VP3 region of any one of SEQ ID NOS:16, 28, 29, 30, or 31. In specific embodiments, the method utilizes a muscle-targeting capsid protein comprises an amino acid sequence of (i) SEQ ID NOS:28, (ii) the VP2 region of SEQ ID NO:28, or (iii) the VP3 region of SEQ ID NO:28. In various embodiments, the method utilizes a first capsid and/or a second capsid that is a liver-targeting capsid. In particular embodiments, the method utilizes a liver-targeting capsid that is selected from the group consisting of Bba.45, Bba.46, Bba.47, Bba.48, Bba.49, Bba.50 and Bba.51. In specific embodiments, the method utilizes a Bba.47 liver-targeting capsid. In specific embodiments, the method utilizes a Bba.49 liver-targeting capsid. In various embodiments, the method utilizes a liver-targeting capsid protein that comprises an amino acid sequence that is at least 95% identical to (i) any one of SEQ ID NOS:158-164, (ii) the VP2 region of any one of SEQ ID NOS:158-164, or (iii) the VP3 region of any one of SEQ ID NOS:158-164. In various embodiments, the method utilizes a liver-targeting capsid that comprises an amino acid sequence of (i) any one of SEQ ID NOS:158-164, (ii) the VP2 region of any one of SEQ ID NOS:158-164, or (iii) the VP3 region of any one of SEQ ID NOS:158-164. In specific embodiments, the method utilizes a liver-targeting capsid protein that comprises an amino acid sequence of (i) SEQ ID NOS:160, (ii) the VP2 region of SEQ ID NO:160, or (iii) the VP3 region of SEQ ID NO:160. In specific embodiments, the method utilizes a liver-targeting capsid protein that comprises an amino acid sequence of (i) SEQ ID NOS:162, (ii) the VP2 region of SEQ ID NO:162, or (iii) the VP3 region of SEQ ID NO:162.

In various embodiments, the subject is administered an immunosuppressant, prior to or subsequent to administration of the second gene therapy vector. In various embodiments, the immunosuppressant is selected from the group consisting of T cell inhibitors, calcineurin inhibitors, mTOR inhibitor and steroids. In various embodiments, the immunosuppressant is anti-thymocyte globulin (ATG), tacrolimus, cyclosporine, mycophenolate mofetil, mycophenolate sodium, azathioprine, sirolimus (rapamycin), or prednisone. In various embodiments, the immunosuppressant is delivered via a delivery vehicle, such as a liposome or nanoparticle.

In various embodiments, the subject is administered intravenous immunoglobulins (IVIG) prior to or subsequent to administration of the second gene therapy vector.

In various embodiments, the second gene therapy vector is administered 6 months, 1 year, 1.5 year, 2 years, 2.5 years, 3 years, 4 years, 5 years or 6 years or more after the first administration of the first gene therapy vector.

The disclosure also contemplates use of an adeno-associated virus (AAV) having a capsid protein having an amino acid sequence that is at least 95% identical to (i) any one of SEQ ID NOs: 15-89 or 158-164, (ii) the VP2 region of any one of SEQ ID NOs: 15-89 or 158-164, or (iii) the VP3 region of any one of SEQ ID NOs: 15-89 or 158-164, and further having a transgene where the transgene is composed of a heterologous gene operably linked to regulatory sequences that control expression of the heterologous gene in a host cell. In another embodiment the capsid protein has the amino acid sequence of (i) any one of SEQ ID NOs: 15-89 or 158-164, (ii) the VP2 region of any one of SEQ ID NOs: 15-89 or 158-164, or (iii) the VP3 region of any one of SEQ ID NOs: 15-89 or 158-164. In yet another embodiment, the AAV has an AAV inverted terminal repeat sequence. In further embodiments, the AAV are mixed with a physiologically compatible carrier.

In various embodiments, the disclosure provides for use of an isolated adeno-associated virus (AAV) capsid protein in the methods, wherein the capsid protein comprises (i) an amino acid sequence that is at least 95%, 96%, 97%, 98%, or 99% identical to the VP1 amino acid sequence of any one of SEQ ID NOS: 15-89 or 158-171 or the VP2 or VP3 region of any one of SEQ ID NOS: 15-89 or 158-164 or (ii) a VP1 amino acid sequence comprising any one of SEQ ID NOS: 15-89 or 158-164 or the VP2 or VP3 region of any one of SEQ ID NOS: 15-89 or 158-164. In certain embodiments, the capsid protein is linked to a heterologous amino acid sequence. The disclosure also provides for non-naturally occurring AAV particles having or comprising any of these capsid proteins. In certain embodiments, the non-naturally occurring AAV particle comprising any of the above described VP1, VP2 or VP3 capsid proteins comprises a nucleic acid having AAV inverted terminal repeats and a transgene comprising a heterologous gene operably linked to regulatory sequences which direct expression of the heterologous gene in a host cell. In other embodiments, the non-naturally occurring AAV particle comprising any of the VP1, VP2 or VP3 capsid sequences described herein comprises a heterologous transgene operably linked to regulatory sequences that control transgene expression in a host cell. As used herein, the terms “heterologous gene” or “heterologous regulatory sequence” means that the referenced gene or regulatory sequence is not naturally present in the AAV vector or particle and is artificially introduced therein. The term “transgene” refers to a nucleic acid that comprises both a heterologous gene and regulatory sequences that are operably linked to the heterologous gene that control expression of that gene in a host cell. It is contemplated that the transgene herein comprises a therapeutic molecule, which can be a therapeutic protein, a therapeutic RNA, an inhibitory RNA (RNAi), mRNA, micro RNA, or a CRISPR/Cas guided endonuclease system.

The disclosure also provides for use of a polynucleotide comprising a nucleotide sequence encoding an adeno-associated virus (AAV) capsid protein, wherein the capsid protein comprises (i) an amino acid sequence that is at least 95%, 96%, 97%, 98% or 99% identical to the VP1 amino acid sequence of any one of SEQ ID NOS: 15-89 or 158-164 or the VP2 or VP3 region of any one of SEQ ID NOS: 15-89 or 158-164 or (ii) a VP1 amino acid sequence comprising any one of SEQ ID NOS: 15-89 or 158-164 or the VP2 or VP3 region of any one of SEQ ID NOS: 15-89 or 158-164, wherein the polynucleotide is operatively linked to a heterologous regulatory control sequence. As such, it is understood that the polynucleotides of described herein are non-naturally occurring. The disclosure also provides for AAV vectors comprising any of these polynucleotide sequences operably linked to a heterologous regulatory sequence and compositions comprising these AAV vectors, including pharmaceutical compositions.

In another embodiment, the disclosure provides an isolated adeno-associated virus (AAV) vector comprising a polynucleotide sequence encoding a capsid protein and a heterologous transgene sequence, wherein the capsid protein comprises (i) an amino acid sequence that is at least 95%, 96%, 97%, 98% or 99% identical to the VP1 amino acid sequence of any one of SEQ ID NOS: 15-89 or 158-164 or the VP2 or VP3 region of any one of SEQ ID NOS: 158-164 or (ii) a VP1 amino acid sequence comprising any one of SEQ ID NOS: 15-89 or 158-164 or the VP2 or VP3 region of any one of SEQ ID NOS: 15-89 or 158-164. The disclosure also provides for compositions comprising these AAV vectors, including pharmaceutical compositions.

In various embodiments, the amino acid sequences of mammalian-derived AAV capsid VP1 proteins useful in the methods herein are set out as SEQ ID NOS:15-89 or 158-164, and the associated locations of the respective VP2 and VP3 sequences are also herein described. In addition, the disclosure provides for novel engineered chimeric AAV capsid proteins which have a backbone amino acid sequence derived from one AAV capsid sequence and fragments of capsid protein sequence derived from at least one different AAV capsid sequence. The amino acid sequences of exemplary engineered chimeric AAV capsid VP1 proteins are set out as SEQ ID NOS: 90-157. Collectively, the capsid proteins are referred to herein as “AAV capsid proteins.” The term “non-naturally occurring” when used in regards to any composition of matter described herein means that the composition is not a product of nature, but rather is artificially synthesized by recombinant or other means. The term “non-naturally occurring” when used in regards to any composition of matter described herein means that the composition is not a product of nature, but rather is artificially synthesized by recombinant or other means.

In another embodiment the disclosure provides for use of a vector and an AAV having a chimeric capsid protein where the chimeric capsid protein has a VP1 amino acid sequence of a recipient backbone AAV capsid having variable regions I, II, III, IV, V, VI, VII, VIII, and IX, except where one or more of the variable regions I, II, III, IV, V, VI, VII, VIII, and IX is replaced by the corresponding variable region from one or more donor AAV capsids. In another embodiment, only one variable region of the recipient capsid is replaced by the corresponding variable region from the donor capsid. In a further embodiment, two or more variable regions of the recipient capsid are replaced by the corresponding variable regions from a single donor AAV capsid. In yet another embodiment, two or more variable regions of the recipient AAV capsid are replaced by the corresponding variable regions from two or more donor AAV capsids. In another embodiment, all nine variable regions of the recipient AAV capsid are replaced by the corresponding variable regions from a single donor capsid. In yet another embodiment the recipient AAV capsid has a GBS region or a GH loop region and the GBS region or the GH loop region is replaced by the corresponding region from one or more donor AAV capsids. In a further embodiment all nine variable regions and the GBS region of the recipient AAV capsid are replaced by the corresponding variable regions and GBS region from one or more donor AAV capsids. In yet another embodiment all nine variable regions and the GBS region of the recipient AAV capsid are replaced by the corresponding regions and GBS region from two or more donor AAV capsids. In another embodiment the GH loop of the recipient AAV capsid is replaced by the corresponding GH loop region from a donor AAV capsid. In a further embodiment all nine variable regions and the GH loop region of the recipient AAV capsid are replaced by the corresponding variable regions and GH loop region from one or more donor AAV capsids. In one embodiment the recipient AAV capsid sequence is any one of SEQ ID NOS:1-14 and the donor AAV capsid sequences are selected from any one of SEQ ID NOS:1-14 and where the recipient AAV capsid and the donor AAV capsid are different. In another embodiment the recipient AAV capsid sequence is any one of SEQ ID NOS:1-89 or 158-164 and the donor AAV capsid sequences are selected from any one of SEQ ID NOS:1-89 or 158-164 and where the recipient AAV capsid and the donor AAV capsid are different. In yet another embodiment the chimeric capsid has the amino acid sequence of any one of SEQ ID NOS:90-157.

In another embodiment, the disclosure provides a method of delivering a transgene to a cell involving the step of contacting the cell with any AAV disclosed herein. In another embodiment, the disclosure provides a method of treating a subject from a disorder or disease associated with abnormal activity of an endogenous protein involving the step of administering to the subject an effective amount of an AAV disclosed herein where the AAV has a transgene that encodes a biologically active copy of the protein, or a transgene that provides a therapeutic polynucleotide such as a mRNA, inhibitory RNA, micro RNA or CRISPR/CAs guide polynucleotide.

In an embodiment, the disclosure provides for use of a composition comprising a vector or AAV disclosed herein for delivery of a transgene to a cell. In another embodiment, the disclosure provides for use of a composition comprising an effective amount of a vector or AAV disclosed herein for the treatment of a disorder or disease associated with abnormal activity of an endogenous protein, wherein the vector of AAV has a transgene that encodes a biologically active copy of a protein useful for treating the disease or disorder, or a transgene that provides a therapeutic polynucleotide such as a mRNA, inhibitory RNA, micro RNA or CRISPR/CAs guide polynucleotide. In certain embodiments, the composition delivers a transgene to a muscle cell or liver cell.

The disclosure also provides for use of a vector or AAV disclosed herein for the preparation of a medicament effective to treat a subject suffering from a disorder or disease associated with abnormal activity of an endogenous protein, wherein the vector or AAV has a transgene that encodes a biologically active copy of the protein as described herein, or a transgene that provides a therapeutic polynucleotide such as a mRNA, inhibitory RNA, micro RNA or CRISPR/CAs guide polynucleotide. In yet another embodiment, the medicament delivers a transgene to a muscle cell or liver cell. In various embodiments, the medicament is useful for redosing a gene therapy vector to treat a disease or disorder set out herein, e.g., hemophilia A, hemophilia B, Wilson's disease, hereditary angioedema (HAE), alpha 1 antitrypsin deficiency, galactosemia, Duchenne's Muscular Dystrophy or other muscular dystrophies, phenylketonuria (PKU), Fabry Disease, and Gaucher Disease.

The disclosure provides for use of fragments of any of the AAV capsid proteins disclosed herein that retain a biological activity of an AAV capsid protein. Exemplary fragments include VP2 and VP3 spliced variants of the capsid proteins, and fragments comprising one or more of the variable regions (VR) of the capsid protein and/or the glycan binding sequence (GBS) of a capsid protein and/or the GH loop. The disclosure also provides for novel, non-naturally occurring AAV particles comprising a capsid protein fragment and those comprising a capsid protein fragment having at least 90, 91, 92, 93, 94, 95, 96, 97, 98 or 99% sequence identity to a specifically defined capsid protein fragment.

Also useful in the methods is the use of chimeric AAV for delivery of and redosing of a gene therapy vector to a subject. AAV VP1 capsid sequences comprise nine different variable regions, a GBS region and a GH loop region, and replacing one of more of these regions in one AAV VP1 capsid sequence with the corresponding region(s) from an at least second, different AAV VP1 capsid sequence can generate chimeric AAV capsids whose associated AAVs are functional, are capable of transducing cells and delivering heterologous transgenes, and that have unique properties that may be recombinantly engineered into the chimeric AAV. In regards to the various variable regions and the GBS and GH loop regions, the term “corresponding” means the same region between two different AAV capsid sequences. For example, the region “corresponding” to VR I in a first AAV capsid sequence is the same region (i.e., the VR I region) in a second different AAV capsid sequence. The term “chimeric” in relation to an AAV capsid sequence refers to the fact that the AAV capsid sequence of interest comprises amino acid sequences derived from two or more different AAV capsid sequences.

As such, the present disclosure also provides for use of an isolated, non-naturally occurring chimeric adeno-associated virus (AAV) capsid protein, wherein the chimeric capsid protein comprises an amino acid sequence derived from a first AAV capsid sequence having at least one variable region substituted with a variable region from a second AAV capsid sequence that is different from the first AAV capsid sequence. The first AAV capsid sequence (referred to herein as the “recipient”) provides the backbone amino acid sequence into which one or more variable regions are swapped or substituted by one or more variable regions from the second AAV capsid sequence (referred to herein as the “donor”). The second AAV capsid sequence is different from the first AAV capsid sequence and will provide the sequence of the variable region(s) which is/are substituted or inserted into the sequence of the backbone or recipient capsid sequence. The disclosure also provides for non-naturally occurring AAV virus or AAV particles that comprise any of the chimeric capsid proteins herein described. In certain embodiments, the non-naturally occurring AAV particles that comprise any of the chimeric capsid proteins herein described also comprise a heterologous transgene operably linked to regulatory sequences that control transgene expression in a host cell.

The “variable regions” refer to the nine variable regions within the VP1 sequence of an AAV capsid protein. Optionally, the variable region may be swapped from a donor AAV capsid sequence into a recipient backbone capsid sequence. The variable regions (VR) are referred to herein as VR I, VR II, VR III VR IV, VR V, VR VI, VR VII, VR VIII and VR IX and their respective locations in various VP1 sequences are herein described. The VR exhibit the highest sequence and structural variation within the AAV VP1 capsid sequence and may also have roles in receptor attachment, transcriptional activation of transgenes, tissue transduction and antigenicity.

The “glycan binding sequence (GBS)” or ‘GBS domain” or “GBS region” refer to the amino acid sequence located between VR IV and VR V that governs the glycan binding specificity of the viral capsid. The locations of the GBS regions in various AAV VP1 amino acid sequences are herein described, and those from other AAV VP1 amino acid sequences are known in the art and/or may be routine identified.

The “GH loop” refers to a loop sequence that is flanked by β-strand G and β-strand H within the internal β-barrel of the capsid protein. The “GH loop” sequence comprises variable region VR IV through VR VIII, including the encompassed GBS sequence and all interspersed conserved backbone sequence from the donor. The locations of the GH loop regions in various AAV VP1 amino acid sequences are herein described and those from other AAV VP1 amino acid sequences may be routinely identified.

In regard to the herein described locations of the VR, GBS and GH loop regions, it is noted that the location of the N-terminal and/or C-terminal ends of those regions may vary by from up to 1 amino acid, 2 amino acids, 3 amino acids, 4 amino acids or 5 amino acids from the amino acid locations of those regions as they are described herein (particularly in Table 4 or Table 5). Capsid sequence comprising substituted VR, GBS and/or GH loop region(s) that vary from up to 5 amino acids on the N-terminal and/or C-terminal end as herein defined are encompassed by the present disclosure.

In one embodiment, the disclosure provides for use of an isolated, non-naturally occurring, chimeric adeno-associated virus (AAV) capsid protein, wherein the chimeric capsid protein comprises an amino acid sequence derived from a first AAV capsid sequence having at least one variable region substituted by a variable region derived from an at least second different AAV capsid sequence. Additional disclosure of chimeric capsids contemplated for use in the methods are set out in the Detailed Description.

The disclosure further provides for methods of producing a recombinant adeno-associated virus (AAV) particle comprising the steps of: culturing a cell that has been transfected with any of the AAV vectors of the invention and recovering recombinant AAV particle from the supernatant of the transfected cell. In addition, the disclosure provides for viral particles comprising any of the viral vectors or capsid proteins of the invention and cells comprising these viral vectors.

One embodiment of the disclosure provides a method of producing any of the recombinant AAV described herein by culturing a viral production cell into which has been introduced a first nucleic acid vector having 5′ and 3′ AAV inverted terminal repeat sequences flanking a transgene having a heterologous gene operably linked to regulatory sequences that control expression of the heterologous gene in a host cell, and a second nucleic acid vector having AAV rep and cap nucleic acids sequences. In various embodiments, said cap nucleic acid sequence encodes an AAV capsid that is at least 95% identical to any of SEQ ID NOs: 15-164; and recovering the AAV from the supernatant of the viral production cell culture. In various embodiments, the viral production cell is a mammalian. In a preferred embodiment the mammalian cell is a HEK293 cell. In various embodiments the viral production cell is an insect cell. In a preferred embodiment the insect cell is an Sf9 cell.

In a further embodiment the first nucleic acid vector is introduced into the viral production cell by infection of the viral production cell by a baculovirus containing the first nucleic acid vector. In yet another embodiment the first and second nucleic acid vectors are introduced into the viral production cell by infection of the viral production cell by a first baculovirus containing the first nucleic acid vector and a second baculovirus containing the second nucleic acid vector. In further embodiments the invention AAV produced by the production methods provided herein.

In another embodiment, the disclosure provides for methods of treating a patient suffering from a disorder or disease comprising administering to the patient an effective amount of any of the AAV vectors or virus described herein.

In a further embodiment, the disclosure provides for use of any of the AAV vectors or virus of the invention for preparation of a medicament for the treatment of a disorder or disease. The invention also provides for compositions comprising any of the AAV vectors or virus of the invention for the treatment of a disease or disorder.

In yet another embodiment, the disease or disorder in a subject is associated with abnormal activity of an endogenous protein. As used herein “endogenous protein” means a protein or gene product encoded by the genome of the subject suffering from the disease or disorder.

An “AAV virion” or “AAV viral particle” or “AAV vector particle” or “AAV virus” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle. As one of ordinary skill in the art would readily appreciate, any AAV referred to herein may be a recombinant AAV (rAAV).

The disclosure also provides for cells comprising any of the AAV vectors described herein, and viral particles produced by these cells.

The term “inverted terminal repeat (ITR)” as used herein refers to the art-recognized regions found at the 5′ and 3′ termini of the AAV genome which function in cis as origins of DNA replication and as packaging signals for the viral genome. AAV ITRs, together with the AAV rep coding region, provide for efficient excision and rescue from a plasmid vector, and integration of a nucleotide sequence interposed between two flanking ITRs into a host cell genome. Sequences of certain AAV-associated ITRs are disclosed by Yan et al., J. Virol. 79(1):364-379 (2005) which is herein incorporated by reference in its entirety.

The phrase “helper functions for generating a productive AAV infection” as used herein refers to AAV-derived coding sequences that can be expressed to provide AAV gene products that, in turn, function in trans for productive AAV replication. Thus, AAV helper functions include the rep and cap regions. The rep expression products have been shown to possess many functions, including, among others: recognition, binding and nicking of the AAV origin of DNA replication; DNA helicase activity; and modulation of transcription from AAV (or other heterologous) promoters. The cap expression products supply necessary packaging functions. AAV helper functions are used herein to complement AAV functions in trans that are missing from AAV vectors. Helper functions for generating a productive AAV infection also may include certain helper functions from baculovirus, herpes virus, adenovirus, or vaccinia virus.

In some embodiments, the viral construct comprises a nucleotide sequence encoding AAV rep and cap genes.

The term “AAV rep gene” as used herein refers to the art-recognized region of the AAV genome which encodes the replication proteins of the virus which are required to replicate the viral genome and to insert the viral genome into a host genome during latent infection. For a further description of the AAV rep coding region, see, e.g., Muzyczka et al., Current Topics in Microbiol. and Immunol. 158:97-129 (1992); Kotin et al., Human Gene Therapy 5:793-801 (1994), the disclosures of which are incorporated herein by reference in their entireties. The rep coding region, as used herein, can be derived from any viral serotype, such as the AAV serotypes described above. The region need not include all of the wild-type genes but may be altered, e.g., by the insertion, deletion or substitution of nucleotides, so long as the rep genes retain the desired functional characteristics when expressed in a suitable recipient cell.

The term “AAV cap gene” as used herein refers to the art-recognized region of the AAV genome which encodes the coat proteins of the virus which are required for packaging the viral genome. For a further description of the cap coding region, see, e.g., Muzyczka et al., Current Topics in Microbiol. and Immunol. 158:97-129 (1992); Kotin et al., Human Gene Therapy 5:793-801 (1994), the disclosures of which are incorporated herein by reference in their entireties. The AAV cap coding region, as used herein, can be derived from any AAV serotype, as described above. The region need not include all of the wild-type cap genes but may be altered, e.g., by the insertion, deletion or substitution of nucleotides, so long as the genes provide for sufficient packaging functions when present in a host cell along with an AAV vector.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual, Cold Spring Harbor Laboratories, New York (1989); Davis et al., Basic Methods in Molecular Biology, Elsevier (1986); Chu et al., Gene 13:197 (1981), the disclosures of which are incorporated herein by reference in their entireties. Such techniques can be used to introduce one or more exogenous DNA moieties, such as a nucleotide integration vector and other nucleic acid molecules, into suitable host cells. The term captures chemical, electrical, and viral-mediated transfection procedures.

The viral construct is, in some embodiments, in the form of a baculoviral vector capable of productive transformation, transfection or infection in any cell type. In some embodiments, the viral construct comprises at least one nucleotide sequence encoding a heterologous protein.

In yet another aspect, described herein is an AAV particle produced by a method described herein. In some embodiments, the AAV particle comprises in its genome at least one nucleotide encoding a heterologous protein.

The term “heterologous proteins or peptides” refer to any protein that is not expressed by wild type AAV including tags such as hexahistidine, FLAG, myc, polyhistidine, or labels or immunogens, adjuvants, selection markers, therapeutic proteins or targeting proteins or peptides, to name a few.

Exemplary heterologous proteins described herein include, but are not limited to, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as α-glucosidase, imiglucarase, β-glucocerebrosidase, and alglucerase; enzyme activators, such as tissue plasminogen activator; chemokines, such as 1β-10, monokine induced by interferon-gamma (Mig), Groa/IL-8, RANTES, MIP-1α, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); Factor VIII, Factor IX, Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; and lysosomal enzymes.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1. In vitro transduction inhibition. This figure shows average pre-existing immunity to different AAV capsids in human subjects, as measured by neutralizing antibody levels in human intravenous immunoglobulin (IVIG). Human IVIG contains pooled IgG from 5000 individuals.

FIG. 2. In vitro transduction inhibition. This figures shows average pre-existing immunity to AAV2, AAV5 and AAV8 capsids compared to a novel isolate Bba.33 capsid in humans, as measured by neutralizing antibody levels in human IVIG.

FIG. 3 shows average pre-existing immunity to AAV12-like capsids compared to AAV5, AAV8 and AAV9 pre-existing immunity in humans, as measured by neutralizing antibody levels in human IVIG.

FIG. 4. This heat map illustrates the levels of pre-existing immunity to different capsids in 50 normal human donors, as measured by in vitro neutralizing titers.

FIG. 5 shows the levels of pre-existing neutralizing antibody titers of non-human primates (NHPs) to different AAV capsids. For each animal, the order of bar graphs, from left to right is Bba.41, AAV2, AAV8, Anc110_9 Vr, AAV9, AAV12 and AAV5.

FIG. 6 represents levels of neutralizing antibodies to Bba.49, in non-human primates(NHPs) pre/post-administration of an AAV5 vector. Bba.49 neutralizing titers measured 2 weeks prior to AAV5 administration (−2), 2 weeks post AAV5 administration, and 7 weeks post AAV5 administration.

FIG. 7 shows levels of neutralizing antibodies to AAV5 in NHPs, pre/post-administration of an AAV5 vector, measured 2 weeks prior to AAV5 administration (−2), 2 weeks post AAV5 administration and 7 weeks post AAV5 administration.

FIG. 8. Pre-existing Nab titer screening of NHPs for study selection; NC50 titers using a NAb neutralization assay. This figure shows levels of pre-existing neutralizing antibodies to AAV9, RN35 and Bba.41 AAV vectors in NHPs.

FIG. 9 illustrates levels of cross-reacting neutralizing antibodies to Bba.41 in NHPs dosed with AAV9, RN35 and Bba.41. Animals from the study summarized in FIG. 8 and the accompanying Example description were selected for use in this study. In particular, animals 1504447, 1504165 and 1410437 were dosed with Bba.49; animals 1505673, 1412911 and 1405945 were dosed with Rn35, and animals 1410197, 1502593 and 1410649 were dosed with AAV9.

FIG. 10 shows levels of cross-reacting neutralizing antibodies to AAV9 in NHPs dosed with AAV9, RN35, and Bba.41. Animals from the study summarized in FIG. 8 and the accompanying Example description were selected for use in this study. In particular, animals 1504447, 1504165 and 1410437 were dosed with Bba.49; animals 1505673, 1412911 and 1405945 were dosed with Rn35, and animals 1410197, 1502593 and 1410649 were dosed with AAV9.

FIG. 11 illustrates neutralizing titers to Bba.49 vector in serum from patients treated with AAV5-FVIII at approximately 1.5-2.5 yrs post-dose.

FIG. 12A-E illustrate neutralizing titers to AAV5, AAV2, AAV6, AAV8 and AAVrh10, respectively, in serum from patients treated with 6e13 vg/kg AAV5-FVIII, prior to AAV dosing, at 8 weeks and approximately 1.5-2.5 yrs post-dose.

FIG. 13A-E illustrate neutralizing titers to AAV5, AAV2, AAV6, AAV8 and AAVrh10, respectively, in serum from patients treated with 4e13 vg/kg AAV5-FVIII, prior to AAV dosing, at 8 weeks and at approximately 1.5-2.5 yrs post-dose.

FIG. 14 illustrates the phylogenetic differences between the AAV vectors and different clades of virus. AAV phylogenetic tree generated with PhyML using MSA input of VP3 sequences from MUSCLE.

FIG. 15A shows levels of bCG transgene expression in mice dosed initially with AAV5-bCG4, then with Bba.47--bCG or Bba.49-bCG four weeks after administration of AAV5-LUC. FIG. 15B shows levels of bCG transgene expression in mice dosed with AAV9-bCG or Bba.41-bCG 4 weeks after administration of AAV9-LUC.

FIG. 16 shows AAV capsid homology between different strains for the VP1 AAV capsid.

FIG. 17 shows AAV capsid homology between different strains for the VP3 AAV capsid.

### DETAILED DESCRIPTION

The disclosure provides for methods of readministration of, or redosing of, gene therapy vectors that minimize the immune response against the second administration of virus elicited by the subject receiving gene therapy. It is hypothesized that administration of a first AAV vector followed by administration of a second AAV vector that varies phylogenetically from the first vector will not be inhibited by any immune response to the first capsid, thereby permitting better transduction efficiency and transgene expression in the subject.

“Readministration” or “redosing” of a gene therapy vector refers to administration to a subject who has previously received at least one gene therapy vector administration to treat a disease or disorder, a second or subsequent administration of another, different gene therapy vector to treat the same disease or disorder. Redosing may refer to multiple doses, i.e., 2, 3, or more doses, of a gene therapy vector.

### AAV Vectors

As used herein, the term “AAV” is a standard abbreviation for adeno-associated virus. Adeno-associated virus is a single-stranded DNA parvovirus that grows only in cells in which certain functions are provided by a co-infecting helper virus. There are numerous serotypes of AAV that have been characterized, examples of which are shown below in Table 1. General information and reviews of AAV can be found in, for example, Carter, Handbook of Parvoviruses, Vol. 1, pp. 169-228 (1989), and Berns, Virology, pp. 1743-1764, Raven Press, (New York, 1990). However, it is fully expected that these same principles will be applicable to additional AAV serotypes since it is well known that the various serotypes are quite closely related, both structurally and functionally, even at the genetic level. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins such as those expressed in AAV6. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes, including encoding a therapeutic protein, RNAi, mRNA, CRISPR/Cas guide polynucleotides) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” or “AAV virus” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

AAV “rep” and “cap” genes are genes encoding replication and encapsidation proteins, respectively. AAV rep and cap genes have been found in all AAV serotypes examined to date, and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap gene in accordance with the present disclosure encodes a Cap protein which is capable of packaging AAV vectors in the presence of rep and adeno helper function and is capable of binding target cellular receptors. In some embodiments, the AAV cap gene encodes a capsid protein having an amino acid sequence derived from a particular AAV serotype, for example the serotypes shown in Table 1.

TABLE 1
AAV serotypes

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

The AAV sequences employed for the production of AAV can be derived from the genome of any AAV serotype. The AAV serotype determines the tissue specificity of infection (or tropism) of an AAV virus. A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences of AAV serotypes). Under the traditional definition, a serotype means that the virus of interest has been tested against serum specific for all existing and characterized serotypes for neutralizing activity and no antibodies have been found that neutralize the virus of interest. As more naturally-occurring virus isolates are discovered and capsid mutants generated, there may or may not be serological differences with any of the currently existing serotypes.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The phylogenetic relationship, distance or similarity between AAVs can be determined, for example, using a Neighbor—Joining algorithm and a Poisson correction distance measurement, based on the alignment of AAV amino acid sequences. The Neighbor—Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV capsid protein, one of skill in the art can readily determine whether a selected AAV is contained in one of the clades identified herein, in another Glade, or is outside these clades. See, e.g., G Gao, et al, J Virol, 2004 June; 78(10): 6381-6388. See, also, WO 2005/033321. The sequence comparisons used to establish phylogenetic relationships between different naturally occurring AAV capsids can also be used to characterize and categorize engineered/synthetic AAV capsids.

The skilled person can select an appropriate serotype, Glade, clone or isolate of AAV for use in the invention on the basis of their common general knowledge.

Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities see, for example, GenBank Accession number U89790; GenBank Accession number J01901; GenBank Accession number AF043303; GenBank Accession number AF085716; Chlorini et al., J. Vir. 71:6823-33(1997); Srivastava et al., J. Vir. 45:555-64 (1983); Chlorini et al., J. Vir. 73:1309-1319 (1999); Rutledge et al., J. Vir. 72:309-319 (1998); and Wu et al., J. Vir. 74: 8635-47 (2000).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a nucleic acid sequence encoding an AAV capsid protein is operably linked to regulatory expression control sequences for expression in a specific cell type, such as Sf9 or HEK cells. Techniques known to one skilled in the art for expressing foreign genes in insect host cells or mammalian host cells can be used to practice the disclosure. Methodology for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. A particularly suitable promoter for transcription of a nucleotide sequence encoding an AAV capsid protein is e.g. the polyhedron promoter. However, other promoters that are active in insect cells are known in the art, e.g. the p10, p35 or IE-1 promoters and further promoters described in the above references are also contemplated.

Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, the nucleic acid construct encoding AAV in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori (Bm)NPV) (Kato et al., Appl. Microbiol. Biotechnol. 85(3):459-470 (2010)). Baculoviruses are commonly used for the infection of insect cells for the expression of recombinant proteins. In particular, expression of heterologous genes in insects can be accomplished as described in for instance U.S. Pat. No. 4,745,051; Friesen et al., Curr. Top. Microbiol. Immunol. 131:31-49. (1986); EP 127,839; EP 155,476; Miller et al., Ann. Rev. of Microbiol. 42: 177-199 (1988); Carbonell et al., Gene 73(2):409-18 (1988); Maeda et al., Nature 315(6020):592-4 (1985); Lebacq-Verheyden et al., Mol. Cell. Biol. 8(8):3129-35 (1988); Smith et al., Proc. Natl. Acad. Sci. USA. 82(24):8404-8 (1985); Miyajima et al., Gene 58(2-3):273-81 (1987); and Martin et al., DNA 7(2):99-106 (1988). Numerous baculovirus strains and variants and corresponding permissive insect host cells that can be used for protein production are described in Luckow et al., Nature Biotechnology 6:47-55 (1988), and Maeda et al., Nature 315(6020):592-4 (1985).

### AAV Capsid Protein

In a first aspect, the disclosure provides for use of AAV capsid proteins that were isolated from various mammalian tissues. The AAV VP1 capsid proteins are provided as set out below and set out in SEQ ID NOs: 15-89 and 158-164 and the locations of the associated VP2 and VP3 regions are described herein.

The disclosure also provides for polynucleotides comprising a nucleotide sequence encoding these novel AAV capsid proteins. The disclosure provides the amino acid sequences of the novel AAV capsid proteins including the engineered chimeric capsid proteins described herein (referred herein collectively as the “AAV capsid proteins of the invention”), and the nucleic acid sequences encoding the AAV capsid proteins of the disclosure. Also provided are fragments of these AAV capsid nucleic acid and amino acid sequences of the disclosure. Each of these sequences may be readily utilized in a variety of vector systems and host cells. Desirable fragments of the capsid VP1 proteins include VP2, VP3 and variable regions, the GBS domain and the GH loop, and polynucleotide sequences encoding these proteins. These fragments may be readily utilized in a variety of vector systems and host cells. Such fragments may be used alone, in combination with other AAV sequences or fragments, or in combination with elements from other AAV or non-AAV viral sequences. In one particular embodiment, a vector contains the AAV capsid sequences described herein.

The AAV capsid sequences of the disclosure and fragments thereof are useful in production of rAAV, and are also useful as antisense delivery vectors, gene therapy vectors, or vaccine vectors. The disclosure further provides nucleic acid molecules, gene delivery vectors, and host cells which contain the novel AAV capsid sequences of the disclosure.

Suitable fragments can be determined using the information provided herein. “Sequence homology” can be determined by performing by alignment of two peptides or two nucleotide sequences using any of a variety of publicly or commercially available Multiple Sequence Alignment Programs, such as “Clustal W”, accessible through Web Servers on the internet. Alternatively, Vector NTI utilities are also used. There are also a number of algorithms known in the art which can be used to measure nucleotide sequence identity, including those contained in the programs described above. As another example, polynucleotide sequences can be compared using FASTA, a program in GCG Version 6.1. FASTA provides alignments and percent sequence identity of the regions of the best overlap between the query and search sequences. For instance, percent sequence identity between nucleic acid sequences can be determined using FASTA with its default parameters (a word size of 6 and the NOPAM factor for the scoring matrix) as provided in GCG Version 6.1, herein incorporated by reference. Similar programs are available for amino acid sequences, e.g., the “Clustal X” program. Additional sequence alignment tools that can be used are provided by (protein sequence alignment; (http://www.ebi.ac.uk/Tools/psa/emboss_needle/)) and (nucleic acid alignment; http://www.ebi.ac.uk/Tools/psa/emboss_needle/nucleotide.html)). Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs.

The terms “substantial identity”, “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences such as 95% identity, 96% identity, 97% identity, 98% identity and 99% identity. Preferably, the homology is over the full-length of the two sequences being compared, or an open reading frame thereof, or another suitable fragment which is at least 15 nucleotides in length. Examples of suitable fragments are described herein. Also included in the nucleic acid sequences of the disclosure are natural variants and engineered modifications of the nucleic acids encoding the AAV capsids of the disclosure and its complementary strand. Such modifications include, for example, labels which are known in the art, methylation, and substitution of one or more of the naturally occurring nucleotides with a degenerate nucleotide.

The terms “substantial identity”, “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences such as 95% identity, 96% identity, 97% identity, 98% identity and 99% identity. Preferably, the homology is over the full-length of the two sequences being compared, or a protein thereof, e.g., the external surface or surface proteins, a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

The term “percent sequence identity” or “identical” in the context of nucleic acid sequences or amino acid sequences refers to the residues in the two sequences which are the same when aligned for maximum correspondence. The length of sequence identity comparison may be over the full-length of the two sequences being compared, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, and may be up to about 700 amino acids. Examples of suitable fragments are described herein.

As described herein, the vectors of the disclosure containing or comprising the AAV capsid proteins are particularly well-suited for use in applications in which the neutralizing antibodies diminish the effectiveness of other AAV serotype based vectors, as well as other viral vectors. The rAAV vectors of the disclosure are particularly advantageous in rAAV re-administration and repeat gene therapy.

Also included within the disclosure are fragments of the nucleic acids encoding the AAV capsid proteins of the disclosure, their complementary strand, cDNA and RNA complementary thereto. Suitable fragments are at least 15 nucleotides in length, and encompass functional fragments, i.e., fragments which are of biological interest. Such fragments include the sequences encoding the three variable proteins (VP) of the capsid which are alternative splice variants: VP1, VP2 and VP3. Other suitable fragments of the nucleic acids encoding the AAV capsids of the disclosure include the fragment which contains the start codon for the capsid protein, and the fragments encoding the variable regions of the VP1 capsid protein, which are described herein.

The disclosure is not limited to the AAV capsid amino acid sequences, peptides and proteins expressed from the AAV nucleic acid sequences of the disclosure and encompasses amino acid sequences, peptides and proteins generated by other methods known in the art, including, e.g., by chemical synthesis, by other synthetic techniques, or by other methods. For example, the sequences of any of the capsids described herein can be readily generated using a variety of techniques.

Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well-known solid phase peptide synthesis methods (Merrifield, J. Am. Chem. Soc., 85:2149 (1962); Stewart and Young, Solid Phase Peptide Synthesis Freeman, (San Francisco, 1969) pp. 27-62. These and other suitable production methods are within the knowledge of those of skill in the art and are not a limitation of the present disclosure.

The AAV capsid is composed of three proteins, VP1, VP2 and VP3, which are alternative splice variants. The full-length capsid sequence is referred to as VP1 which encompasses the spliced variants referred to as VP2 and VP3. The disclosure also provides for other functional fragments of the AAV capsid proteins of the disclosure. Other desirable fragments of the capsid protein include the variable regions (VR), the constant regions which are located between the variable regions, the GBS domain, and the GH loop. Other desirable fragments of the capsid protein include the HPV themselves.

An algorithm has been developed to determine areas of sequence divergence in AAV2. (Chiorini et al, J. Virol, 73:1309-19 (1999); Rutledge et al, J. Virol., 72:309-319 (1998)). Using this algorithm and/or the alignment techniques described herein, the VR of the AAV capsid sequences are determined. Using the alignment provided herein performed using the Clustal X program at default settings, or using other commercially or publicly available alignment programs at default settings, one of skill in the art can readily determine corresponding fragments of the novel AAV capsids of the disclosure.

Suitably, fragments of an AAV capsid protein are at least 8 amino acids in length, or at least 9 amino acids in length, or at least 10 amino acids in length, or least 20 amino acids in length, or 30 amino acids in length or at least 50 amino acids in length, or at least 75 amino acids in length, or at least 100 amino acids in length or 200 amino acids in length or 250 amino acids in length or 300 amino acids in length or 350 amino acids in length or 400 amino acids in length. However, fragments of other desired lengths may be readily utilized. All fragments of the disclosure retain biological activity of a capsid AAV protein. Such fragments may be produced recombinantly or by other suitable means, e.g., chemical synthesis.

The sequences, proteins, and fragments of the disclosure may be produced by any suitable means, including recombinant production, chemical synthesis, or other synthetic means. Such production methods are within the knowledge of those of skill in the art and are not a limitation of the present disclosure.

In addition to including the nucleic acid sequences provided in the Sequence Listing, the present disclosure includes nucleic acid molecules and sequences which are designed to express the amino acid sequences, proteins and peptides of the AAV capsid proteins of the disclosure. Thus, the disclosure includes nucleic acid sequences which encode the following AAV capsid amino acid sequences and artificial AAV capsid proteins generated using these sequences and/or unique fragments thereof.

Artificial capsid or engineered capsid proteins may be generated by any suitable technique, using a AAV capsid protein sequence of the disclosure (e.g., a fragment of a VP1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

### Chimeric AAV Capsid

In one embodiment, the method contemplates use of an isolated, non-naturally occurring, chimeric adeno-associated virus (AAV) capsid protein, wherein the chimeric capsid protein comprises an amino acid sequence derived from a first AAV capsid sequence having at least one variable region substituted by a variable region derived from an at least second different AAV capsid sequence. In certain embodiments, the non-naturally occurring capsid protein is a VP1 capsid protein. In other embodiments, the chimeric capsid protein further comprises a GBS domain and/or a GH loop region from an AAV capsid sequence differing from the first recipient AAV capsid sequence. For example, the chimeric AAV capsid proteins of the disclosure have a backbone sequence derived from a first AAV capsid sequence (recipient) and at least one substituted variable region from a second different AAV capsid sequence (donor). In certain embodiments, the chimeric AAV capsid proteins of the present disclosure have one, two, three, four, five, six, seven, eight or all nine variable regions substituted by the respective variable region(s) from one or more donor AAV capsid sequence(s) that differ from the first recipient capsid sequence. In other embodiments, the AAV capsid proteins of the disclosure have a GBS domain or GH loop region sequence derived from a donor capsid sequence that differs from the recipient capsid sequence. Alternatively, the chimeric AAV capsids of the disclosure have at least one substitute variable region and a GBS from the same AAV capsid sequence which differs from the first AAV capsid sequence. The disclosure also provides non-naturally occurring AAV particles comprising any of the chimeric AAV capsid proteins described herein. Such AAVs may also comprise a heterologous transgene operably linked to a regulatory sequence controlling expression of the transgene in a host cell.

In addition, the disclosure provides for use of isolated AAV capsid proteins, wherein the capsid protein comprises an amino acid sequence from a first AAV capsid sequence which has two variable regions substituted with the respective variable regions from at least one AAV capsid sequence that differs from the first AAV capsid sequence, or least three variable regions substituted with the respective variable regions from at least one AAV capsid sequence that differs from the first AAV capsid sequence, or least four variable regions substituted with the respective variable regions from at least one AAV capsid sequence that differs from the first AAV capsid sequence, or least five variable regions substituted with the respective variable regions from at least one AAV capsid sequence that differs from the first AAV capsid sequence, or least six variable regions substituted with the respective variable regions from at least one AAV capsid sequence that differs from the first AAV capsid sequence, or least seven variable regions substituted with the respective variable regions from at least one AAV capsid sequence that differs from the first AAV capsid sequence, or least eight variable regions substituted with the respective variable regions from at least one AAV capsid sequence that differs from the first AAV capsid sequence, or all nine of the variable regions substituted with the respective variable regions from at least one AAV capsid sequence that differs from the first AAV capsid sequence. For example, the substituted variable region(s) are from the same AAV capsid sequence or the substituted variable regions are from two or more different AAV capsid sequences that differ from the first AAV capsid sequence. In addition, in any of these AAV capsid proteins of the disclosure, the GBS and/or the GH loop are also substituted and may be derived from any AAV donor capsid sequence that differs from the first AAV capsid sequence.

In any of the chimeric AAV capsids of the disclosure, the first/recipient AAV capsid sequence and the second/donor AAV capsid sequence can be any known or herein described AAV capsid sequence including, for example, capsid sequences associated with the following AAV sequences: AAV-1, AAV-2, AAV-3, AAV-3B, AAV-4, AAV-5, AAV-6, AAV-7, AAV-8, AAV-9, AAV-10, AAV-11, AAV-12, AAV-13, AAVbo, AAVmo, AAV6.2, AAVRH.8, AAV4.10, AAVanc80L65 or AAVanc110, or any of the other AAV serotypes or capsid sequences herein described. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014).

In certain embodiments, the chimeric AAV capsid proteins of the disclosure may comprise the amino acid sequence of any one of SEQ ID NOS:90-157 (see Table 7, below), each of which have at least one variable region from a donor AAV serotype swapped for the respective variable region(s) in the recipient backbone sequence.

In any of the chimeric AAV capsid proteins of the disclosure, the backbone sequence or the amino acid sequence from the first AAV capsid sequence derives from the amino acid sequence of any of SEQ ID NO:1-89, e.g., 1-73 or 15-89, or 158-164. In addition, in any of the chimeric AAV capsid proteins of the disclosure, the donor sequence or the amino acid sequence from the second AAV serotype derives from the amino acid sequence of one or more variable regions, GBS domain and/or GH loop of any of SEQ ID NO:1-89, e.g., 1-73 or 15-89, or 158-164.

In another embodiment, the disclosure provides for an isolated polynucleotide sequence comprising a nucleotide sequence encoding any of the engineered chimeric AAV capsid proteins of the disclosure. In addition, the disclosure provides for isolated AAV vectors comprising these polynucleotide sequences and AAV vectors comprising a polynucleotide sequence encoding any of the chimeric AAV capsid proteins of the disclosure. The disclosure also provides for compositions comprising these AAV vectors, including pharmaceutical compositions. The disclosure also provides for use of AAV virus comprising any of the herein described non-naturally occurring chimeric AAV capsid proteins.

Exemplary capsids include AAV1, AAV2, AAV4, AAV3, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, AAV listed in Table 6, chimeric AAV listed in Table 7 and/or variants thereof.

### Capsid Pairs for Redosing

In various embodiments, it is contemplated that the subject receiving gene therapy is administered a gene therapy vector comprising a first AAV capsid, and if necessary, the subject may be administered a second dose of gene therapy comprising administration of a gene therapy vector comprising a second AAV capsid that is different from the first AAV capsid administered to the subject. The first and second AAV capsid can be referred to as “capsid pairs”

AAV has been divided into six different clades, A-F (Gao et al., J Virol 78:6381-6388, 2004) based on genome analysis and homology or diversity of different strains of AAV to other identified strains. AAV strains having higher homology are categorized in the same clade, and believed to derive from a similar lineage. According to Gao et al. (supra), most AAV strains belong to a different clade, though AAV1 and AAV6 appear to belong to the same clade as do AAV2 and AAV4.

It is contemplated that members of capsid pairs (or first and second capsids) are phylogenetically diverse and possess a limited amount of sequence homology. For example in various embodiments, the phylogenetic difference or diversity is based on a threshold level of sequence homology. In various embodiments, the capsid pairs are categorized in different clades. In another embodiment, the capsid pairs are derived from AAV that infect different hosts, e.g., human, baboon or other non-human primate, goats, ungulates and other animal which are infected by an AAV strain.

In various embodiments, the capsid pairs exhibit a sequence homology difference in one or more shared antibody binding epitopes. For example, the capsid pairs (or first and second capsids) have homology differences in two or more, three or more, four or more antibody epitopes found in the AAV capsid. In various embodiments, the sequence homology difference is at a threshold level that minimizes antibody cross-reactivity between the two capsids.

In various embodiments, a threshold level of sequence homology is approximately less than or equal to 90% sequence homology over the capsid amino acid sequence, or over any one of the VP1, VP2 or VP3 capsid proteins. In various embodiments, the first and second capsids (capsid pairs) have amino acid sequence homology that is less than or equal to about 90%. In various embodiments, the first and second capsids have less than or equal to about 90% homology in a VP1 capsid protein, have less than or equal to about 90% homology in a VP2 capsid protein and/or less than or equal to about 90% homology in a VP3 capsid protein. In various embodiments, the sequence homology of the capsids, or capsid proteins, may be less than or equal to 90%, 89%, 88%, 87%, 86%, 85%, 84%, 83%, 82%, 81%, 80%, 79%, 78%, 77%, 76%, 75% or lower. In various embodiments, the sequence homology of the capsids, or capsid proteins, may be from about 30% to 90% homologous, from about 45% to 87% homologous, from about 40% to 86% homologous, from about 50% to 85% homologous, or from about 60% to 80% homologous, or from about 65% to 75% homologous.

The decreased homology between the two capsid proteins limits the cross-reactive immune response to the second capsid that may be generated by the subject after receiving a second gene therapy dose.

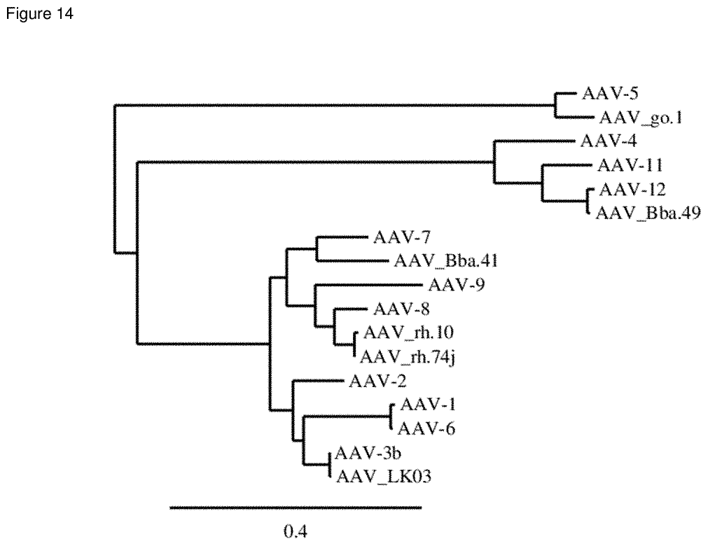

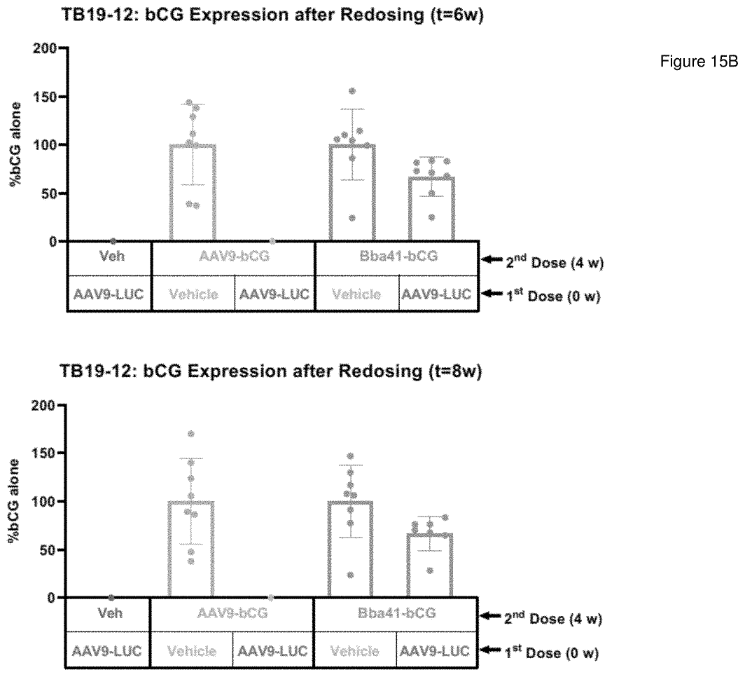

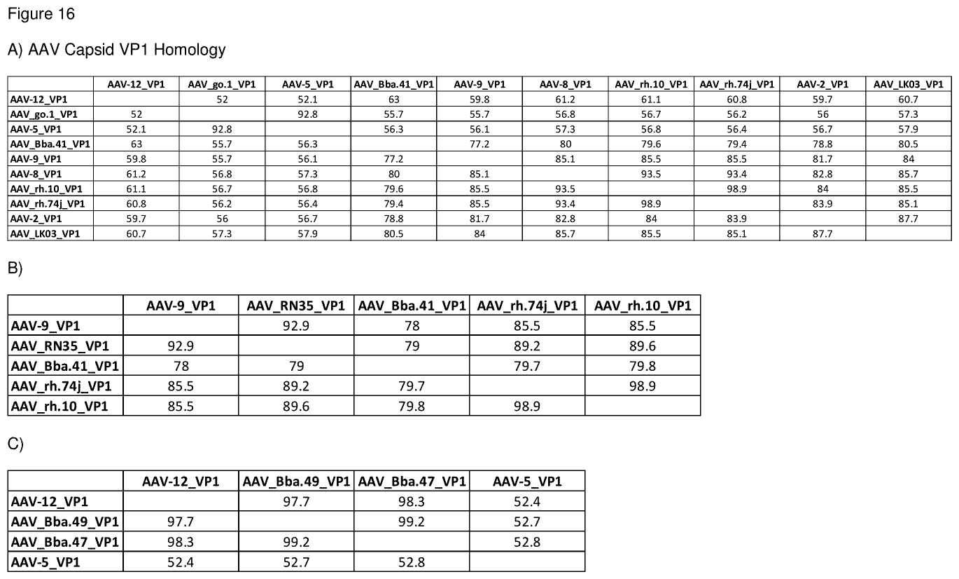

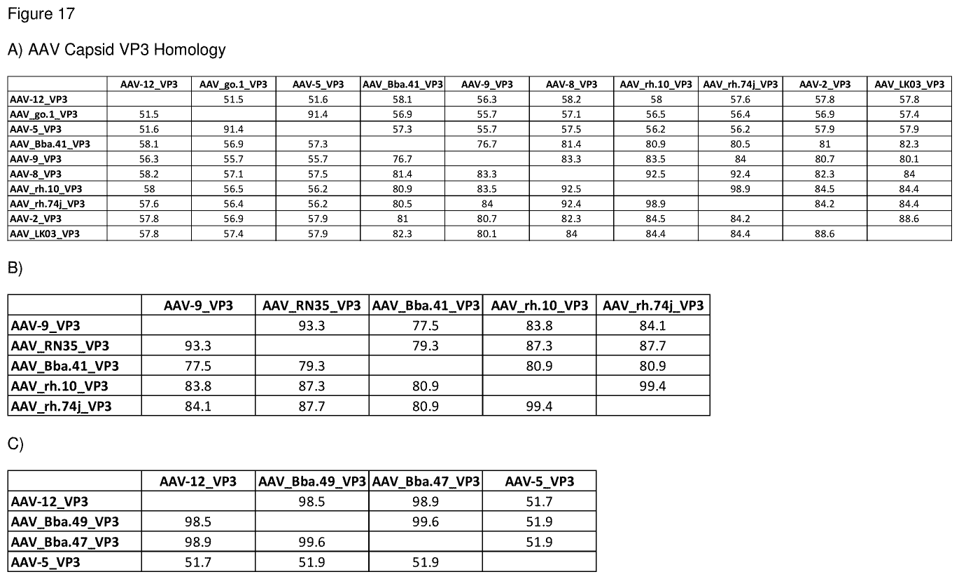

For example, the percent identities of selected AAV vector capsids is set out in Table 2 and FIGS. 16 and 17. An exemplary phylogenetic tree based on sequence homology of VP3 regions is set out in FIG. 14.

TABLE 2
Capsid Protein Percent Homology

| | AAV5 | Bba.49 | Bovine | Bba.41 |
|---|---|---|---|---|
| AAV5 | | 55 | 56 | 59 |
| Bba.49 | 55 | | 77 | 65 |
| Bovine | 56 | 77 | | 60 |
| Bba.41 | 59 | 65 | 60 | |
| AAV9 | 59 | 63 | 59 | 79 |
| Rh74 | 58 | 64 | 60 | 81 |
| LK03 | 60 | 63 | 60 | 82 |
| AAV8 | 59 | 63 | 59 | 82 |
| Rh10 | 59 | 64 | 60 | 81 |

In a non-limiting example, capsid pairs are set out in Table 3, wherein the First Capsid can be paired with any one of the AAV listed as a Second Capsid. In a similar manner, any AAV listed as a second capsid could be administered as a first capsid, provided a different capsid is administered as the second capsid.

TABLE 3
Exemplary Capsid Pairs

| First Capsid | Second Capsid |
|---|---|
| AAV5 | Bba.49, bovine |
| LK03 | AAV5, Bba.49, bovine |
| AAV8 | AAV5, Bba.49, bovine |
| Rh10 | AAV5, Bba.49, bovine |

As described herein, the first and second capsids of a capsid pair for use in the redosing methods described herein are different from each other. With this in mind, in various embodiments, the first and second capsid is selected from the group consisting of AAV5, Bba.49, Bba.47 and bovine. In various embodiments, the first and second capsid proteins comprise an amino acid sequence that is at least 95% identical to (i) to SEQ ID NOS: 1, 5, 160 or 162, (ii) the VP2 region of any one of SEQ ID NOS: 1, 5, 160 or 162, or (iii) the VP3 region of any one of SEQ ID NOS: 1, 5, 160 or 162. In various embodiments, the first and second capsid proteins comprise an amino acid sequence of (i) any one of SEQ ID NOS:1, 5, 160 or 162, (ii) the VP2 region of any one of SEQ ID NOS: 1, 5, 160 or 162, or (iii) the VP3 region of any one of SEQ ID NOS: 1, 5, 160 or 162. In various embodiments, the first and second capsid proteins comprise an amino acid sequence of any one of SEQ ID NOS:1, 5, 160 or 162.

In various embodiments, the first and second capsid is selected from the group consisting of LK03, AAV5, Bba.49 and bovine. In various embodiments, the first and second capsid proteins comprise an amino acid sequence that is at least 95% identical to (i) to SEQ ID NOS: 1, 5, 162 or 173, (ii) the VP2 region of any one of SEQ ID NOS: 1, 5, 162 or 173, or (iii) the VP3 region of any one of SEQ ID NOS: 1, 5, 162 or 173. In various embodiments, the first and second capsid proteins comprise an amino acid sequence of (i) any one of SEQ ID NOS: 1, 5, 162 or 173, (ii) the VP2 region of any one of SEQ ID NOS: 1, 5, 162 or 173, or (iii) the VP3 region of any one of SEQ ID NOS: 1, 5, 162 or 173. In various embodiments, the first and second capsid proteins comprise an amino acid sequence of any one of SEQ ID NOS: 1, 5, 162 or 173.

In various embodiments, the first and second capsid is selected from the group consisting of AAV8, AAV5, Bba.49 and bovine. In various embodiments, the first and second capsid proteins comprise an amino acid sequence that is at least 95% identical to (i) to SEQ ID NOS: 1, 5, 9 or 162, (ii) the VP2 region of any one of SEQ ID NOS: 1, 5, 9 or 162, or (iii) the VP3 region of any one of SEQ ID NOS: 1, 5, 9 or 162. In various embodiments, the first and second capsid proteins comprise an amino acid sequence of (i) any one of SEQ ID NOS: 1, 5, 9 or 162, (ii) the VP2 region of any one of SEQ ID NOS: 1, 5, 9 or 162, or (iii) the VP3 region of any one of SEQ ID NOS: 1, 5, 9 or 162. In various embodiments, the first and second capsid proteins comprise an amino acid sequence of any one of SEQ ID NOS: 1, 5, 9 or 162.

In various embodiments, the first and second capsid is selected from the group consisting of rh10, AAV5, Bba.49 and bovine. In various embodiments, the first and second capsid proteins comprise an amino acid sequence that is at least 95% identical to (i) to SEQ ID NOS: 1, 5, 12 or 162, (ii) the VP2 region of any one of SEQ ID NOS: 1, 5, 12 or 162, or (iii) the VP3 region of any one of SEQ ID NOS: 1, 5, 12 or 162. In various embodiments, the first and second capsid proteins comprise an amino acid sequence of (i) any one of SEQ ID NOS: 1, 5, 12 or 162, (ii) the VP2 region of any one of SEQ ID NOS: 1, 5, 12 or 162, or (iii) the VP3 region of any one of SEQ ID NOS: 1, 5, 12 or 162. In various embodiments, the first and second capsid proteins comprise an amino acid sequence of any one of SEQ ID NOS: 1, 5, 12 or 162.

In certain embodiments, the first capsid protein and second capsid protein are from two different clades. In some embodiments, the first capsid protein and the second capsid protein are from the same clade but have sequence homology that is approximately less than or equal to 90% sequence homology over any one, two or thereof the VP1, VP2 or VP3 capsid proteins.

It is also contemplated herein that if a third or subsequent dose of gene therapy is required to re-express a transgene of interest in a subject, yet another distinct AAV capsid is used to redoes the therapy or readminister the transgene, and the third or subsequent vector is distinct from the first or second vector previously administered to the subject.

In various embodiments, neutralizing antibodies to the first capsid will not interfere with transduction of the 2^{nd} capsid. In various embodiments, administration of the second AAV capsid permits increased transduction efficiency in the subject compared to transduction levels after a second administration of a vector comprising the same first AAV capsid.

In various embodiments, the first capsid and second capsid exhibit low pre-existing immunity in the subject.

In various embodiments, the subject is human. In various embodiments, the subject is human and is immunologically naïve to the first and second AAV vector.

Colella et al., (Mol Ther Methods Clin Dev. 8: 87-104, 2018) discusses certain issues in AAV therapy, and describes that NAb titers as low as ˜1:5 can block transduction of the liver following AAV8-FIX vector administration in non-human primates. In various embodiments, the subject has neutralizing titers less than 1:2, 1:5, 1:10, 1:20, 1:50, 1:100, 1:200 or 1:300 to the first or second AAV vector in serum.

In certain embodiments, the subject with low pre-existing immunity has less than 1:2, 1:5, or 1:10 anti-first AAV vector neutralizing antibody titer or less than 1:100 total anti-first AAV vector-IgG titer in a sample (e.g., blood, sera, or plasma) from the subject as assessed by a technique described herein or known to one of skill in the art, such as, e.g., Meadows et al., Mol Ther Methods Clin Dev. 13: 453-462, 2019. In certain embodiments, the subject with low pre-existing immunity has less than 1:2, 1:5 or 1:10 anti-second AAV vector neutralizing antibody titer or less than 1:100 total anti-second AAV vector-IgG titer. In some embodiments, the subject with low pre-existing immunity has less than 1:10 anti-first AAV vector neutralizing antibody or less than 1:20, 1:50, 1:80, 1:100, 1:200, 1:300, 1:400, or 1:500 total anti-first AAV vector-IgG titer in a sample (e.g., blood, sera, or plasma) from the subject as assessed by a technique described herein or known to one of skill in the art, such as, e.g., Meadows et al., Mol Ther Methods Clin Dev. 13: 453-462, 2019. In certain embodiments, the subject with low pre-existing immunity has less than 1:10 anti-second AAV vector neutralizing antibody titer or less than 1:10, 1:20, 1:50, 1:80, 1:100, 1:200, 1:300, 1:400, or 1:500 total anti-second AAV vector-IgG titer (Meadows et al., Mol Ther Methods Clin Dev. 13: 453-462, 2029).

In some embodiments, the subject with low pre-existing immunity has an NC50 titer of anti-first AAV vector neutralizing antibody of less than 320, 312, 310, 300, 275, 250, 200, 175, 150, 125, 100, 75, 50, 30 or 25 in a sample (e.g., blood, sera, or plasma) from the subject as assessed by a technique described herein (e.g., in the Example section). In certain embodiments, the subject with low pre-existing immunity has an NC50 titer of anti-second AAV vector neutralizing antibody of less than 320, 312, 310, 300, 275, 250, 200, 175, 150, 125, 100, 75, 50, 30 or 25 in a sample (e.g., blood, sera, or plasma) from the subject as assessed by a technique described herein (e.g., in the Example section).

In various embodiments, the neutralizing antibody levels are measured in a neutralizing antibody assay. Methods to detect pre-existing AAV immunity include cell-based in vitro TI assays, in vivo (eg, mice) TI assays, and enzyme-linked immunosorbent assay (ELISA)-based detection of total anticapsid antibody (TAb) assays. (Masat et al., Discov Med 2013 15:379-389; Boutin et al., Hum Gene Ther 2010 21:704-712). The TAb assay may be able to detect low potency NAb that are below the threshold of TI assays, but it may not detect non-antibody neutralizing factors. In vivo and in vitro TI assays screen samples for anti-AAV Nab (Manno et al., Nat Med 2006, 12:342-347, Boutin et al., Hum Gene Ther 2010; 21: 704-712, Calcedo et al. Clin Vaccine Immunol 2011; 18: 1586-1588, Mingozzi et al., Gene Ther 2013; 20: 417-424, Calcedo et al., J Infect Dis 2009; 199: 381-390, Halbert et al., Hum Gene Ther 2006; 17: 440-447, Li et al., Gene Ther 2012; 19: 288-294, Moskalenko et al., J Virol 2000; 74: 1761-1766, Wang et al. Mol Ther 2010; 18: 126-134, Grimm et al., J Virol 2008; 82: 5887-5911, Greenberg et al. Gene Ther 2016; 23: 313-319, Meliani et al., Hum Gene Ther Methods 2015; 26: 45-53, Sun et al., J Immunol Methods 2013; 387: 114-120) and other factors that modulate AAV transduction efficiency (Berry et al. Mol Ther 2016; 24(Suppl 1): S14 (abstract 30), Weinberg et al. J Virol 2014; 88: 12472-12484, Hirosue et al. Virology 2007; 367: 10-18, Viral-Lowell et al., Gene Ther 2000; 7: 1783-1789, Mitchell et al. J Virol 2013; 87: 13035-13041, Mitchell et al., J Virol 2013; 87: 4571-4583, Berry et al. J Biol Chem 2016; 291: 939-947, Nonnenmacher et al. Gene Ther 2012; 19: 649-658).

In some embodiments, anti-first capsid protein antibody(ies) present in a sample (e.g., blood, sera or plasma) from a subject does not significantly cross-react with a second capsid protein if there is no detectable binding of the antibody(ies) to the second capsid protein as assessed by techniques known in the art, e.g., ELISA, Western blot, biolayer interferometry, FACS or BIACore. In certain embodiments, anti-first capsid antibody(ies) present in a sample (e.g., blood, sera or plasma) from a subject does not significantly cross-react with a second capsid if the antibody(ies) has a 5-fold, 10-fold, 15-fold, 20-fold, 25-fold or greater-fold affinity for the first capsid protein than the second capsid protein as assessed by techniques known in the art, e.g., ELISA, Western blot, biolayer interferometry, FACS or BIACore. In some embodiments, anti-second capsid protein antibody(ies) present in a sample (e.g., blood, sera or plasma) from a subject does not significantly cross-react with a first capsid protein if there is no detectable binding of the antibody(ies) to the first capsid protein as assessed by techniques known in the art, e.g., ELISA, Western blot, biolayer interferometry, FACS or BIACore. In certain embodiments, anti-second capsid antibody(ies) present in a sample (e.g., blood, sera or plasma) from a subject does not significantly cross-react with a first capsid if the antibody(ies) has a 5-fold, 10-fold, 15-fold, 20-fold, 25-fold or greater-fold affinity for the second capsid protein than the first capsid protein as assessed by techniques known in the art, e.g., ELISA, Western blot, biolayer interferometry, FACS or BIACore.

In some embodiments, anti-first AAV vector antibody(ies) present in a sample (e.g., blood, sera or plasma) from a subject does not significantly cross-react with a second AAV vector if there is no detectable binding of the antibody(ies) to the second AAV vector as assessed by techniques known in the art, e.g., ELISA, Western blot, biolayer interferometry, flow cytometry or BIACore, or described herein. In certain embodiments, anti-first AAV vector antibody(ies) present in a sample (e.g., blood, sera or plasma) from a subject does not significantly cross-react with a second AAV vector if the antibody(ies) has a 5 fold, 10 fold, 15 fold, 20 fold, 25 fold or greater fold affinity for the first AAV vector than the second AAV vector as assessed by techniques known in the art, e.g., ELISA, Western blot, biolayer interferometry, flow cytometry or BIACore. In some embodiments, anti-second AAV vector antibody(ies) present in a sample (e.g., blood, sera or plasma) from a subject does not significantly cross-react with a first AAV vector if there is no detectable binding of the antibody(ies) to the first AAV vector as assessed by techniques known in the art, e.g., ELISA, Western blot, biolayer interferometry, flow cytometry or BIACore, or described herein. In certain embodiments, anti-second AAV vector antibody(ies) present in a sample (e.g., blood, sera or plasma) from a subject does not significantly cross-react with a first AAV vector if the antibody(ies) has a 5 fold, 10 fold, 15 fold, 20 fold, 25 fold or greater fold affinity for the second AAV vector than the first AAV vector as assessed by techniques known in the art, e.g., ELISA, Western blot, biolayer interferometry, flow cytometry or BIACore. In a specific embodiment, cross-reactivity between a first capsid protein and a second capsid protein is determined as set forth in an Example provided herein.

In various embodiments, the redosing methods described herein may be utilized with one or more tissue-targeting capsid proteins. To determine the tissue specific infectivity of the AAV capsids disclosed herein, methods such as those described in WO2018/022608 or WO2019/222136, each of which is incorporated herein in its entirety and in particular for its tissue specific AAV infectivity assays and disclosure. Briefly, AAV comprising a test capsid and expressing one or more detectable transgenes, for example a luciferase transgene (e.g., a Fluc or Fluc2 gene) and/or a green fluorescent protein (GFP) transgene, may be generated and tested in animals, e.g., Balb/C mice, by introducing the AAV into the test animals at one or more concentrations and at an appropriate time post-infection (e.g., at 3 and 5 weeks post-infection) measurement, for example imaging, of the detectable marker or markers may be performed.

In the case of a luciferase marker, for example, in vivo bioluminescent imaging may be employed, utilizing standard bioluminescent substrates and imaging devices. Whole animal imaging and/or organ imaging may be used. Image data may be processed and analyzed using software, for example, living image software version 4.5.2 (PerkinsElmer Waltham, Mass.). Regions of interest may be traced surrounding each animal as well as individual organs to quantify the total flux (TF) (photons/second) being released by luciferase activity. Total flux activity is a proxy for AAV infectivity of each organ system.

With respect to muscle specificity, one method to classify a capsid as muscle-specific, is to calculate at the ratio of gastrocnemius flux/liver flux. If this ratio is greater than a specified ratio, e.g. a 2-fold increase in flux, and the flux in no other non-muscle tissue is greater than the flux in liver, the capsid protein may be characterized as muscle-specific. With respect to liver specificity, one method to classify a capsid as liver-specific is to associate a capsid with at least a 2-fold increase, for example at least a 5-10-fold increase, in liver flux relative to the other tissues tested.

Tissue specific infectivity imparted by a capsid may also be assessed, for example, by utilizing a GFP transgene, whereby tissue from infected infected test animals, e.g., mice, may be harvested and sectioned, and the percent of cells expressing GFP may be quantitated for different tissues or organs, for example, muscle or liver tissue or organs.

Utilizing assays such as described hereinabove, and as demonstrated in WO2018/022608 and/or WO2019/222136, AAV capsid proteins Bba.45, Bba.46, Bba.47, Bba.49, Bba.50 and Bba.51 have been identified as liver-tropic, that is, ingas exhibit a high degree of liver-specificity, while AAV capsid proteins AAVancl 10_9 VR, Bba.26, Bba41, Bba.42, Bba.43 and Bba.44 have been identified as muscle-tropic, that is, as exhibiting a high degree of muscle-specificity.

Production of AAV with the Capsid Proteins

The disclosure encompasses AAV capsid protein sequences and the nucleic acids encoding these proteins of which are free of DNA and/or cellular material which these viruses are associated in nature. In another aspect, the present disclosure provides molecules which utilize the novel AAV sequences of the disclosure, including fragments thereof, for production of molecules useful in delivery of a heterologous gene or other nucleic acid sequences to a target cell.

In another aspect, the present disclosure provides molecules which utilize the AAV capsid protein sequences of the disclosure, including fragments thereof, for production of viral vectors useful in delivery of a heterologous gene or other nucleic acid sequences to a target cell.

The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

In one embodiment, the vectors of the disclosure contain, at a minimum, sequences encoding the AAV capsid of the disclosure or a fragment thereof. In another embodiment, the vectors of the disclosure contain, at a minimum, sequences encoding an AAV rep protein or a fragment thereof. Optionally, such vectors may contain both AAV cap and rep proteins. In vectors in which both AAV rep and cap are provided, the AAV rep and AAV cap sequences can both be of the same AAV serotype origin. Alternatively, the present disclosure provides vectors in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector. Optionally, the vectors further contain a minigene comprising a selected transgene which is flanked by AAV 5′ ITR and AAV 3′ ITR.

Thus, in one embodiment, the vectors described herein contain nucleic acid sequences encoding an intact AAV capsid protein of any one of amino acid sequences SEQ ID 1-89, e.g., 1-73 or 15-89, or 158-164. Alternatively, these vectors contain sequences encoding artificial capsids which contain one or more fragments of the capsid in SEQ ID NOs: 1-89, e.g., 1-73 or 15-89, or 158-164 fused to heterologous AAV or non-AAV capsid proteins (or fragments thereof). These artificial capsid proteins are selected from non-contiguous portions of the any of the AAV capsid proteins of the invention or from capsids of other AAV serotypes.

In another example, it may be desirable to alter the start codon of the VP3 protein to GTG. Alternatively, the rAAV may contain one or more of the variable regions of one or more of the AAV capsid proteins of the disclosure, or other fragments. These modifications may be to increase expression, yield, and/or to improve purification in the selected expression systems, or for another desired purpose (e.g., to change tropism or alter neutralizing antibody epitopes).

The vectors described herein, e.g., a plasmid, are useful for a variety of purposes, but are particularly well suited for use in production of a rAAV containing a capsid comprising AAV sequences or a fragment thereof. These vectors, including rAAV, their elements, construction, and uses are described in detail herein.

Capsid Proteins from Baboon Liver

AAV VP1 capsid proteins isolated from baboon liver are disclosed in co-owned PCT Application No. PCT/US19/32097, which published as WO2019/222136, and which is incorporated herein by reference in its entirety.

The VP1 sequence of an AAV capsid isolated from baboon (denoted as Bba.45) is set out as SEQ ID NO:158 (amino acids 1-742) and the locations of the associated variable regions and GBS and GH loop regions are defined in Table 4 below. The VP2 capsid protein spans amino acids 138-742 of SEQ ID NO:158 and the VP3 capsid protein spans amino acids 206-742 of SEQ ID NO:158.

(SEQ ID NO: 158)
MAADGYLPDWLEDNLSEGIREWWALKPGAPQPKANRQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRALDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVREVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.46) is set out as SEQ ID NO: 159 (amino acids 1-742) and the locations of the associated variable regions and GBS and GH loop regions are defined in Table 4 below. The VP2 capsid protein spans amino acids 138-742 of SEQ ID NO: 159 and the VP3 capsid protein spans amino acids 206-742 of SEQ ID NO: 159.

(SEQ ID NO: 159)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGESTPWGCFDENREHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGAATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.47) is set out as SEQ ID NO: 160 (amino acids 1-742) and the locations of the associated variable regions and GBS and GH loop regions are defined in Table 4 below. The VP2 capsid protein spans amino acids 138-742 of SEQ ID NO: 160 and the VP3 capsid protein spans amino acids 206-742 of SEQ ID NO: 160.

(SEQ ID NO: 160)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.48) is set out as SEQ ID NO: 161 (amino acids 1-742) and the locations of the associated variable regions and GBS and GH loop regions are defined in Table 4 below. The VP2 capsid protein spans amino acids 138-742 of SEQ ID NO: 161 and the VP3 capsid protein spans amino acids 206-742 of SEQ ID NO: 161.

(SEQ ID NO: 161)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QERLQEDTSFGGNLGRAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCRFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTAITTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNAATAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.49) is set out as SEQ ID NO: 162 (amino acids 1-742) and the locations of the associated variable regions and GBS and GH loop regions are defined in Table 4 below. The VP2 capsid protein spans amino acids 138-742 of SEQ ID NO: 162 and the VP3 capsid protein spans amino acids 206-742 of SEQ ID NO: 162.

(SEQ ID NO: 162)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QERLQEDTSFGGNLGRAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTAITTYGKITTGDFAYYRKNWLPGAGIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.50) is set out as SEQ ID NO: 163 (amino acids 1-742) and the locations of the associated variable regions and GBS and GH loop regions are defined in Table 4 below. The VP2 capsid protein spans amino acids 138-742 of SEQ ID NO: 163 and the VP3 capsid protein spans amino acids 206-742 of SEQ ID NO: 163.

(SEQ ID NO: 163)
MAADGYLPDWLEDNLSESIREWWALKPGAPRPKANQQHQDDARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGRKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPLHSMYAHSQSLDRMMNPLLDQYLWHLQST
TTGNSLNQGTATTTYGKITTGDFAYYRKNWLPGACIKQQKFSKNASQNYK
IPASGEDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.51) is set out as SEQ ID NO: 164 (amino acids 1-742) and the locations of the associated variable regions and GBS and GH loop regions are defined in Table 4 below. The VP2 capsid protein spans amino acids 138-742 of SEQ ID NO: 164 and the VP3 capsid protein spans amino acids 206-742 of SEQ ID NO: 164.

(SEQ ID NO: 164)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The corresponding nucleic acid sequences encoding the above referenced capsid proteins are set out as follows: SEQ ID NO:165/Bba.45; SEQ ID NO:166/Bba.46; SEQ ID NO:167/Bba.47; SEQ ID NO:168/Bba.48; SEQ ID NO:169/Bba.49; SEQ ID NO:170/Bba.50; and SEQ ID NO:171/Bba.51.

In Table 4 below, “VR” refers to the variable region and the numbers refer to the amino acid residues of each variable region or the GBS and GH loop regions in the amino acid sequence.

TABLE 4

| AAV | VRI | VRII | VRIII | VRIV | GBS | VRV | VRVI | VRVII | VRVIII | VRIX | GHloop |
|---|---|---|---|---|---|---|---|---|---|---|---|
| AAVBba.45 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.46 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.47 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.48 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.49 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.50 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |
| AAVBba.51 | 265- | 325- | 380- | 452- | 467- | 490- | 534- | 550- | 587- | 710- | 448- |
| | 268 | 330 | 386 | 462 | 478 | 512 | 544 | 564 | 599 | 718 | 607 |

Capsid Proteins from AAV

Capsid proteins isolated from tissue from the following mammals: baboon, crab-eating macaque, cynomolgus macaque, marmoset and pig useful in the methods of described herein are also described in co-owned International Patent Publication No. WO 2018/022608, incorporated herein by reference.

Contemplated for use in the methods are engineered chimeric AAV capsid proteins (and AAV comprising those capsid proteins) in which one or more variable region(s), the GBS region and/or the GH loop in a backbone (or recipient) capsid protein sequence are substituted with one or more variable region(s), GBS region and/or GH loop from a different AAV capsid sequence donor. The recipient and donor sequences may derive from any previously known AAV serotype or capsid sequence, or any novel AAV capsid sequence described herein. The engineered AAV capsid proteins are generated by swapping at least one variable region, GBS region or GH loop region from one capsid sequence for the respective region(s) in a recipient capsid sequence. In this regard, it is noted that one, two, three, four, five, six, seven, eight or all nine VRs in a recipient VP1 capsid sequence can be replaced by the respective region(s) from one or more different VP1 capsid sequence. Any and all of the various combinations of engineered, chimeric AAV capsid sequences that can be produced by the VR region swapping method described herein (and all associated AAV virus comprising those chimeric capsid sequences) are contemplated herein.

The VP1 sequence of an AAV capsid isolated from baboon (denoted as Bba.21) is set out as SEQ ID NO:15 (amino acids 1-742) and the locations of the associated variable regions and GBS and GH loop regions are defined in Table 5 below. The VP2 capsid protein spans amino acids 138-742 of SEQ ID NO:15 and the VP3 capsid protein spans amino acids 206-742 of SEQ ID NO:15.

(SEQ ID NO: 15)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGESTPWGYFDENREHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEVSYQFEKVPFHSMYAHSQSLDRMMNPLLDQYLWHPQST
TTGNSLNQGTATTTYGKITTGDFAYYRKNWLPGACIKQQKFSKNASQNYK
IPASGGDALLKYDTHTTSNGRWSNMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYLGPIWAKVPHTDGHFHPSPLMGGFGLKHPP
PQIFIKNTPVPANPNTTFSAARINSFLTQYSTGQVAVQIDWEIQKEHSKR
WDPEVQFTSNYGTQNSMLWAPDNAGNYHEPRAIGSRFLTHHL

The VP1 sequence of an AAV capsid isolated from baboon (denoted as Bba.26) is set out as SEQ ID NO:16 (amino acids 1-739) and the locations of the associated variable regions and GBS and GH loop regions are defined in Table 5 below. The VP2 capsid protein spans amino acids 138-739 of SEQ ID NO:16 and the VP3 capsid protein spans amino acids 206-739 of SEQ ID NO:16.

(SEQ ID NO: 16)
MAADGYLPDWLEDNLSEGIREWWALKPGAPQPKANQQHQGNSRGLVLPGY
KYLGLFNGLDKGEPVNEADAAALEHDKAYDKQLEQGDDPYLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFGPQPAAPIGQPPAAPPP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNVQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLVTSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.27) is set out as SEQ ID NO:17 (amino acids 1-739) and the locations of the associated variable regions and GBS and GH loop regions are defined in Table 5 below. The VP2 capsid protein spans amino acids 138-739 of SEQ ID NO:17 and the VP3 capsid protein spans amino acids 206-739 of SEQ ID NO:17.

(SEQ ID NO: 17)
VAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEL
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPPP
VGSNTMASGGGGPMADDNQGADGVGNASGNWHRDSTWLGDRVITTSTRTW
VLPTYNNHIYKQISSESGATNDNHYEGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
SQGILPGMVWQDRDVYLQGPIWAKIPHTDGHFHPSPLMGGFGLKHPPPQI
LIKNTPVPANPPETFTTSKFASYITQYSTGQVSVEIEWELQKENGKRWNP
EIQYTSNYAKSNNVEFSVDAAGVYSEPRPIGTRYLTRNL

The VP1 sequence of an AAV capsid isolated from baboon (denoted as Bba.29) is set out as SEQ ID NO:18 (amino acids 1-739) and the locations of the associated variable regions and GBS and GH loop regions are defined in Table 5 below. The VP2 capsid protein spans amino acids 138-739 of SEQ ID NO:18 and the VP3 capsid protein spans amino acids 206-739 of SEQ ID NO:18.

(SEQ ID NO: 18)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAKTAPGKRRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYEGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQCLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKEDSKRWNP
EIQYTSNYAKSNNVEFSVDAAGVYSEPRPIGTRYLTRNL

The VP1 sequence of an AAV capsid isolated from baboon (denoted as Bba.30) is set out as SEQ ID NO:19 (amino acids 1-739) and the locations of the associated variable regions and GBS and GH loop regions are defined in Table 5 below. The VP2 capsid protein spans amino acids 138-739 of SEQ ID NO:19 and the VP3 capsid protein spans amino acids 206-739 of SEQ ID NO:19.

(SEQ ID NO: 19)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAKTAPGKKRPLEKTP
NQPTDTQAAGQTPAKKRPQGGQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YLLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKEDSKRWNP
EIQYTSNYAKSNNVEFSVDAAGVYSEPRPIGTRYLTRNL

The VP1 sequence of an AAV capsid isolated from baboon (denoted as Bba.31) is set out as SEQ ID NO:20 (amino acids 1-742) and the locations of the associated variable regions and GBS and GH loop regions are defined in Table 5 below. The VP2 capsid protein spans amino acids 138-742 of SEQ ID NO:20 and the VP3 capsid protein spans amino acids 206-742 of SEQ ID NO:20.

(SEQ ID NO: 20)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKHPLEKTP
NRPTNPDSGKAPAKKKQKDGETADSARRTLDFEDSGAGDGPPEGSSSGEM
SHDAEMRAAPGGNAVEAGQGADGVGNASGDWHCGSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLECF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLFTSEEEIAATNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPDNAGNYHEPRAIGSRFLTHHL

The VP1 sequence of an AAV capsid isolated from baboon (denoted as Bba.32) is set out as SEQ ID NO:21 (amino acids 1-742) and the locations of the associated variable regions and GBS and GH loop regions are defined in Table 5 below. The VP2 capsid protein spans amino acids 138-742 of SEQ ID NO:21 and the VP3 capsid protein spans amino acids 206-742 of SEQ ID NO:21.

(SEQ ID NO: 21)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGKAPAKKKQKDGETADSARRTLDFEDSGAGDGPPEGSSSGEM
SHDAEMRAAPGGNAVEAGQGADGVGNASGGWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSSMAPGPPMATAGAGDSDFSNSQLIFAG
PNQSGNTTTSSNNLLFTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPGNAGNHHEPRAIGSRFLTHHL

The VP1 sequence of an AAV capsid isolated from baboon (denoted as Bba.33) is set out as SEQ ID NO:22 (amino acids 1-742) and the locations of the associated variable regions and GBS and GH loop regions are defined in Table 5 below. The VP2 capsid protein spans amino acids 138-742 of SEQ ID NO:22 and the VP3 capsid protein spans amino acids 206-742 of SEQ ID NO:22.

(SEQ ID NO: 122)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEF
QQRLATDTSFGGNLGKAVFQAKKRILEPLGLVEEGVKTAPGKKRPLEKTP
NRPTNPDSGRAPAKKKQKDGETADSARRTLDFEDSGAGDGPPEGSSSGEM
SHDAEMRAAPGGNAVEAGQGADGVGNASGDWHCDSTWSEGRVTTTSTRTW
VLPTYNNHLYLRIGTTANSNTYNGFSTPWGYFDFNRFHCHFSPRDWQRLI
NNNWGLRPKSMRVKIFNIQVKEVTTSNGETTVANNLTSTVQIFADSTYEL
PYVMDAGQEGSLPPFPNDVFMVPQYGYCGVVTGENQNQTDRNAFYCLEYF
PSQMLRTGNNFEISYQFEKVPFHSMYAHSQSLDRMMNPLLDQYLWHLQST
TTGNSLNQGTATTTYGKITTGDFAYYRKNWLPGACIKQQKFSKNASQNYK
IPASGGDALLKYDTHTTLNGRWSNMAPGPPMATAGAGDSDFSNSQLIFAG
PNQSGNTTTSSNNLLLTSEEEIATTNPRDTDMFGQIADNNQNATTAPHIA
NLDAMGIVPGMVWQNRDIYYQGPIWAKVPHTDGHFHPSPLMGGFGLKHPP
PQIFIKNTPVPANPNTTFSAARINSFLTQYSTGQVAVQIDWEIQKEHSKR
WNPEVQFTSNYGTQNSMLWAPGNAGNYHEPRAIGSRFLTHHL

The VP1 sequence of an AAV capsid isolated from baboon (denoted as Bba.34) is set out as SEQ ID NO:23 (amino acids 1-739) and the locations of the associated variable regions and GBS and GH loop regions are defined in Table 5 below. The VP2 capsid protein spans amino acids 138-739 of SEQ ID NO:23 and the VP3 capsid protein spans amino acids 206-739 of SEQ ID NO:23.

(SEQ ID NO: 23)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGILGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLVNPGIAMATHKDDEERFFPQHGVLVFGKT
NATNKTTLENVLVTDEEEVKATNPVATEEYGTVSSNLQSNTTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSADAAGVYSEPRPIGTRYLTRNL

The VP1 sequence of an AAV capsid isolated from baboon (denoted as Bba.35) is set out as SEQ ID NO:24 (amino acids 1-739) and the locations of the associated variable regions and GBS and GH loop regions are defined in Table 5 below. The VP2 capsid protein spans amino acids 138-739 of SEQ ID NO:24 and the VP3 capsid protein spans amino acids 206-739 of SEQ ID NO:24.

(SEQ ID NO: 24)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEVAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHRVLVFGQT
NATNKTTLDNVLITSEEEIKATNPVATEEYGTVSSNLQASNTDPTTETVN
NQGILPGMVWQDRDVYLQGPIWVKIPHTDGHFHPSPLMGGFGLKHPPPQI
LIKNTPVPANPPETFTTSKFASYTTQYSTGQVSVEIEWGLQKENSKRWNP
EIQYTSNYAKSNNVEFSVDAAGVYSEPRPIGTRYLTRNL

The VP1 sequence of an AAV capsid isolated from baboon (denoted as Bba.36) is set out as SEQ ID NO:25 (amino acids 1-739) and the locations of the associated variable regions and GBS and GH loop regions are defined in Table 5 below. The VP2 capsid protein spans amino acids 138-739 of SEQ ID NO:25 and the VP3 capsid protein spans amino acids 206-739 of SEQ ID NO:25.

(SEQ ID NO: 25)
MAADGYLPDWLEDNLSEGIREWWALKPGAPQPKANQQHQDNSRGLVLPGY
KYLGPFNGLDKGEPVNEAYAAALEHDKAYDKQLEQGDNPYLKYNHADAEF
QERLQEDTSFGGNLGRAVFQAKKRIPEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRPSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.37) is set out as SEQ ID NO:26 (amino acids 1-739) and the locations of the associated variable regions and GBS and GH loop regions are defined in Table 5 below. The VP2 capsid protein spans amino acids 138-739 of SEQ ID NO:26 and the VP3 capsid protein spans amino acids 206-739 of SEQ ID NO:26.

(SEQ ID NO: 26)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAETAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQTEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIINPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.38) is set out as SEQ ID NO:27 (amino acids 1-739) and the locations of the associated variable regions and GBS and GH loop regions are defined in Table 5 below. The VP2 capsid protein spans amino acids 138-739 of SEQ ID NO:27 and the VP3 capsid protein spans amino acids 206-739 of SEQ ID NO:27.

(SEQ ID NO: 27)
MAADGYLPDWLEDNLSEGIRGWWALKPGAPQPKANQQHQDNSRGLVLPGY
KYLGPFNGLDKGEPVNEADAAALEHDKAYDKQLEQGDNPCLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPTWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.41) is set out as SEQ ID NO:28 (amino acids 1-739) and the locations of the associated variable regions and GBS and GH loop regions are defined in Table 5 below. The VP2 capsid protein spans amino acids 138-739 of SEQ ID NO:28 and the VP3 capsid protein spans amino acids 206-739 of SEQ ID NO:28.

(SEQ ID NO: 28)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNSFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLANPGIAMATHKDDEERFFPQHGVLVFGQT
NATNKTTLDNVLITSEEEIKATNPVATEEYGTVSSNLQASNTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.42) is set out as SEQ ID NO:29 (amino acids 1-739) and the locations of the associated variable regions and GBS and GH loop regions are defined in Table 5 below. The VP2 capsid protein spans amino acids 138-739 of SEQ ID NO:29 and the VP3 capsid protein spans amino acids 206-739 of SEQ ID NO:29.

(SEQ ID NO: 29)
MAADGYLPDWLEDNLSEGIREWWALKPGAPQPKANQQHQDNSRGLVLPGY
KYLGPFNGLDKGEPVNGADAAALEHDKAYDKQLEQGDNPYLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYEGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLVNPGIAMATHKDDEERFFPQHGVLVFGKT
NATNKTTLENVLVTDEEEVKATNPVATEEYGTVSSNLQSNTTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRLIGTRYLTRNL

The VP1 sequence of an AAV capsid isolated from baboon (denoted as Bba.43) is set out as SEQ ID NO:30 (amino acids 1-739) and the locations of the associated variable regions and GBS and GH loop regions are defined in Table 5 below. The VP2 capsid protein spans amino acids 138-739 of SEQ ID NO:30 and the VP3 capsid protein spans amino acids 206-739 of SEQ ID NO:30.

(SEQ ID NO: 30)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQN
SNTNFAWTAASKYNLNGRKSLVNPGIAMATHKDDEERFFPQHGVLVFGKT
NATNKTTLENVLVTDEEEVKATNPVATEEYGTVSSNLQSNTTNPTTETVN
NQGILPGMVWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from baboon (denoted as Bba.44) is set out as SEQ ID NO:31 (amino acids 1-739) and the locations of the associated variable regions and GBS and GH loop regions are defined in Table 5 below. The VP2 capsid protein spans amino acids 138-739 of SEQ ID NO:31 and the VP3 capsid protein spans amino acids 206-739 of SEQ ID NO:31.

(SEQ ID NO: 31)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEF
QERLQEDTSFGGNLGRAVFQAKKRILEPLGLVEEAAKTAPGKKRPLEKTP
NQPTDTQAAGQTPAKKRPQGEQSGDSARRQLDFDPQPAAPIGQPPAAPSP
VGSNTMASGGGGPMADDNQGADGVGNASGNWHCDSTWLGDRVITTSTRTW
VLPTYNNHIYKQISSESGATNDNHYFGYSTPWGYFDFNRFHCHFSPRDWQ
RLINNNWGFRPKRLNFKIFNIQVKEVTQIEGGSTIANNLTSTIQVFADSE
YQLPYVLGSAHQGCLPPFPADVFMIPQYGYLTLNNGSQSVGRSSFYCLEY
FPSQMLRTGNNFQFSYTFESVPFHSSYAHSQSLDRIMNPLVDQYLYYLAR
TQTGTGSTTSNTRQLQFYQAGPSNMADQSRNWLPGPMYRQQRVSKTLDQD
SNTNFAWTAASKYNLNGRKSLVNPGIAMATHKDDEERFFPQHGVLVFGKT
NATNKTTLENVLVTDEEEVKATNPVATEEYGTVSSNLQSNTTNPTTETVN
NQGILPGMAWQDRDVYLQGPIWAKIPHTDGHFHPSPLMGGFGLKHPPPQI
LIKNTPVPANPPETFTTSKFASYITQYSTGQVSVEIEWELQKENSKRWNP
EIQYTSNYAKSNNVEFSVDAAGVYSEPRPIGTRYLTRNL

The VP1 sequence of an AAV capsid isolated from crab-eating macaque (denoted as Bce.14) is set out as SEQ ID NO:32 (amino acids 1-736) and the locations of the associated variable regions and GBS and GH loop regions are defined in Table 5 below. The VP2 capsid protein spans amino acids 138-736 of SEQ ID NO:32 and the VP3 capsid protein spans amino acids 203-736 of SEQ ID NO:32.

(SEQ ID NO: 32)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTG
NNFTFSYEFENVPFHSSYAHSQSLDRLMNPLIDQYLYYLSKTINGSGQNQQ
ALKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLVGGFGMKHPPPQILIKNTPVPADPPTAFN
KDKLNSFITQYSTGQVRVEIEWELQKENSKRWNPEIQYTSNYYKSNNVEFA
VNTEGVYSEPRPIGTRYLTRNL

The VP1 sequence of an AAV capsid isolated from crab-eating macaque (denoted as Bce.15) is set out as SEQ ID NO:33 (amino acids 1-736) and the locations of the associated variable regions and GBS and GH loop regions are defined in Table 5 below. The VP2 capsid protein spans amino acids 138-736 of SEQ ID NO:33 and the VP3 capsid protein spans amino acids 203-736 of SEQ ID NO:33.

(SEQ ID NO: 33)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMPRTG
NNFTFSYEFENVPFHSSYAHSQSLDRLMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVAIEWELQKENSKRWNPEIQYTSNYYKSNNVEFA
VNTEGVYSEPRPIGTRYLTRNL

The VP1 sequence of an AAV capsid isolated from crab-eating macaque (denoted as Bce.16) is set out as SEQ ID NO:34 (amino acids 1-736) and the locations of the associated variable regions and GBS and GH loop regions are defined in Table 5 below. The VP2 capsid protein spans amino acids 138-736 of SEQ ID NO:34 and the VP3 capsid protein spans amino acids 203-736 of SEQ ID NO:34.

(SEQ ID NO: 34)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTG
NNFTFSYEFENVPFHSSYAHSQSLDRPMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVEIEWELQKENSKRWNPEIQYTSNYYKSNNVEFA
VNTEGVYSEPRPIGTRYLTRNL

The VP1 sequence of an AAV capsid isolated from crab-eating macaque (denoted as Bce.17) is set out as SEQ ID NO:35 (amino acids 1-736) and the locations of the associated variable regions and GBS and GH loop regions are defined in Table 5 below. The VP2 capsid protein spans amino acids 138-736 of SEQ ID NO:35 and the VP3 capsid protein spans amino acids 203-736 of SEQ ID NO:35.

(SEQ ID NO: 35)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAIKRLNFGQTGDTESVPDPQPIGEPPAAPSGVGSLTMA
SGGGAPVADNNEGADGVGSSSGNWHCDSQWLGDRVITTSIRTWALPTYNNH
LYKQISNSTSGGSSNDNAYFGYSTPWGYFDFSRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTG
NNFTFSYEFENVPFHSSYAHSQSLDRLMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVEIEWELQKENSKRWNPEIQYTSNYYKSNNVEFA
VNTEGVYSEPRPIGTRYLTRNL

The VP1 sequence of an AAV capsid isolated from crab-eating macaque (denoted as Bce.18) is set out as SEQ ID NO:36 (amino acids 1-736) and the locations of the associated variable regions and GBS and GH loop regions are defined in Table 5 below. The VP2 capsid protein spans amino acids 138-736 of SEQ ID NO:36 and the VP3 capsid protein spans amino acids 203-736 of SEQ ID NO:36.

(SEQ ID NO: 36)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTG
NNFTFSYEFENVPFHSSYAHSQSLDRLMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVEIEWELQKENSKRWNPEIQYTSNYYKSNNVEFA
VNTEGVYSEPRPIGTRYLTRNL

The VP1 sequence of an AAV capsid isolated from crab-eating macaque (denoted as Bce.20) is set out as SEQ ID NO:37 (amino acids 1-733) and the locations of the associated variable regions and GBS and GH loop regions are defined in Table 5 below. The VP2 capsid protein spans amino acids 138-733 of SEQ ID NO:37 and the VP3 capsid protein spans amino acids 203-733 of SEQ ID NO:37.

(SEQ ID NO: 37)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSRWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGAKTIANNLTSTVQVFTDSDYQLPYVLG
SAHEGCLPPFPADVFMIPQYGYLTLNDGSQAVGRSSFYCLEYFPSQMLRTG
NNFTFSYEFENVPFHSSYAHSQSLDRLMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVEIEWELQKENSKRWNPEIQYTSNYYKSNNVEFA
VNTEGVYSEPRPIGTRYLT

The VP1 sequence of an AAV capsid isolated from crab-eating macaque (denoted as Bce.35) is set out as SEQ ID NO:38 (amino acids 1-730) and the locations of the associated variable regions and GBS and GH loop regions are defined in Table 5 below. The VP2 capsid protein spans amino acids 138-730 of SEQ ID NO:38 and the VP3 capsid protein spans amino acids 199-730 of SEQ ID NO:38.

(SEQ ID NO: 38)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVLQAKKRVLEPLGLVEEAAKTAPGKKRPVDSPDSTS
GIGKKGQQPARKRLNFGQTGDAESVPDPQPIGEPPAAPSGLGSGTMAAGGG
APMADNNEGADGVGNASGNWHCDSTWLGNRVITTSTRTWALPTYNNHLYKQ
ISSSSSGATNDNHYFGYSTPWGYFDFNRFHCHFSPRDWQRLINNNWGFRPK
RLRFKLFNIQVKEVTTNDGVTTIANNLTSTVQVFSDSEYQLPYVLGSAHQG
CLPPFPADVFMIPQYGYLTLNNGSQSVGRSSFYCLEYFPSQMLRTGNNFEF
SYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRS
SLTNPGVPMATHKDDESVFFPINGVLVFGKTGASNKTTLENVLMTDEEEIK
ATNPVATEEYGVVSSNIQSQNSNPTTQTVNNQGALPGMVWQNRDVYLQGPI
WAKIPHTDGNFHPSPLMGGFGLKHPPPQILIKNTPVPANPPETFTPAKFAS
FITQYSTGQVSVEIEWELQKENSKRWNPEIQYTSNYDKQTGVGFAVDTQGV
YSEPRPIGTRYLTRNL

The VP1 sequence of an AAV capsid isolated from crab-eating macaque (denoted as Bce.36) is set out as SEQ ID NO:39 (amino acids 1-730) and the locations of the associated variable regions and GBS and GH loop regions are defined in Table 5 below. The VP2 capsid protein spans amino acids 138-730 of SEQ ID NO:39 and the VP3 capsid protein spans amino acids 199-730 of SEQ ID NO:39.

(SEQ ID NO: 39)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVDSPDSTS
GIGKKGQQPARKRLNFGQTGDAESVPDPQPIGEPPAAPSGLGSGTMAAGGG
APMADNNEGADGVGNASGNWHCDSTWLGNRVITTSTRTWALPTYNNHLYKQ
ISSSSSGATNDNHYFGYSTPWGYFDFNRFHCHFSPRDWQRLINNNWGFRPK
RLRFKLFNIQVKEVTTNDGVTTIANNLTSTVQVFSDSEYQLPYVLGSAHQG
CLPPFPADVFMIPQYGYLTLNNGSQSVGRSSFYCLEYFPSQMLRTGNNFEF
SYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRN
SLTNPGVPMATHKDDESVFFPINGVLVFGKTGASNKTTLENVLMTDEEEIK
ATNPVATEEYGVVSSNIQSQNSNPTTQTVNNQGALPGMVWQNRDVYLQGPI
WAKIPHTDGNFHPSPLMGGFGLKHPPPQILIKNTPVPANPPETFTPAKFAS
FITQYSTGQVSVEIEWELQKENSKRWNPEIQYTSNYDKQTGVDFAVDTQGV
YSEPRPIGTRYLTRNL

The VP1 sequence of an AAV capsid isolated from crab-eating macaque (denoted as Bce.39) is set out as SEQ ID NO:40 (amino acids 1-736) and the locations of the associated variable regions and GBS and GH loop regions are defined in Table 5 below. The VP2 capsid protein spans amino acids 138-736 of SEQ ID NO:40 and the VP3 capsid protein spans amino acids 203-736 of SEQ ID NO:40.

(SEQ ID NO: 40)
MAADGYLPDWLEDNLSEGIREWWDLKPGAPKPKANQQKQDDGRGLVLPGYK
YPGPFNGLDKGEPVNAADAAALEHDKAYDQQLKAGDNPYLRYNHADAEFQE
RLQEDTSFGGNLGRAVFQAKKRVFEPLGLVEEGAKTAPGKKRPVEQSPQEP
DSSSGIGKTGQQPAKKRLNFGQTGDSESVPDPQPLGEPPAAPSGLGPNTMA
SGGGAPMADNNEGADGVGNSSGNWHCDSTWLGDRVITTSTRTWALPTYNNH
LYKQISNSTSGGSTNDNTYFGYSTPWGYFDFNRFHCHFSPRDWQRLINNNW
GFRPKRLNFKLFNIQVKEVTTNEGTKTIANNLTSTVQVFTDSEYQLPYVLG
SAHQGCLPPFPADVFMVPQYGYLTLNNGSQALGRSSFYCLEYFPSQMLRTG
NNFQFSYTFEDVPFHSSYAHSQSLDRLMNPLIDQYLYYLVRTQTTGTGGTQ
TLAFSQAGPSSMANQARNWVPGPSYRQQRVSTTKNQNNNSNFAWTGAAKFK
LNGRNSLMNPGVAMASHKDDEDRFFPSSGVLIFGKQGAGNDGVDYSQVLIT
DEEEIKATNPVATEEYGEVAINDQAANTQAQTGLVHNQGVIPGMVWQNRDV
YLQGPIWAKIPHTDGNFHPSPLMGGFGLMHPPPQILIKNTPVPADPPLTFN
QAKLNSFITQYSTGQVSVEIEWELQKENSKRWNPEIQYTSNYYKSTNVDFA
VNSDGVYSEPRPIGTRYLTRNL

The VP1 sequence of an AAV capsid isolated from crab-eating macaque (denoted as Bce.40) is set out as SEQ ID NO:41 (amino acids 1-736) and the locations of the associated variable regions and GBS and GH loop regions are defined in Table 5 below. The VP2 capsid protein spans amino acids 138-736 of SEQ ID NO:41 and the VP3 capsid protein spans amino acids 203-736 of SEQ ID NO:41.

(SEQ ID NO: 41)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQG
RLQEDTSFGGNLGRAVFQAKKRVLEPLGLVEEGAKTAPGKKRPVEQSPQEP
DSSSGIGKTGQQPAKKRLNFGQTGDSESVPDPQPLGEPPAAPSGLGPNTMA
SGGGAPMADNNEGADGVGNSSGNWHCDSTWLGDRVITTSTRTWALPTYNNH
LYKQISNSTSGGSTNDNTYFGYSTPWGYFDFNRFHCHFSPRDWQRLINNNW
GFRPKRPNFKLFNIQVKEVTTNEGTKTIANNLTSTVQAFTDSEYQLPYVLG
SAHQGCLPPFPADVFMVPQYGYLTLNNGSQALGRSSFYCLEYFPSQMLRTG
NNFQFSYTFEDVPFHSSYAHSQSLDRLMNPLIDQYLYYLVRTQTTGTGGTQ
TLAFSQAGPSSMANQARNWVPGPSYRQQRVSTTKNQNNNSNFAWTGAAKFK
LNGRNSLMNPGVAMASHKDDEDRFSPSSGVLIFGKQGAGNDGVDYSQVLIT
DEEEIKATNPVATEEYGEVAINDQAANTQAQTGLVHNQGVIPGMVWQNRDV
YLQGPIWAKIPHTDGNFHPSPLMGGFGLKHPPPQILIKNTPVPADPPLTFN
QAKLNSFITQYSTGQVSVEIEWELQKENSKRWNPEIQYTSNYYKSTNVDFA
VNSDGVYSEPRPIGTRYLTRNL

The VP1 sequence of an AAV capsid isolated from crab-eating macaque (denoted as Bce.41) is set out as SEQ ID NO:42 (amino acids 1-736) and the locations of the associated variable regions and GBS and GH loop regions are defined in Table 5 below. The VP2 capsid protein spans amino acids 138-736 of SEQ ID NO:42 and the VP3 capsid protein spans amino acids 203-736 of SEQ ID NO:42.

(SEQ ID NO: 42)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQG
RLQEDTSFGGNLGRAVFQAKKRVLEPLGLVEEGAKTAPGKKRPVEQSPQEP
DSSSGIGKTGQQPAKKRLNFGQTGDSESVPDPQPLGEPPAAPSGLGPNTMA
SGGGAPMADNNEGADGVGNSSGNWHCDSTWLGDRVITTSTRTWALPTYNNH
LYKQISNSTSGGSTNDNTYFGYSTPWGYFDFNRFHCHFSPRDWQRLINNNW
GFRPKRLNFKLFNIQVKEVTTNEGTKTIANNLTSTVQVFTDSEYQLPYVLG
SAHQGCLPPFPADVFMVPQYGYLTLNNGSQALGRSSFYCLEYFPSQMLRTG
NNFQFSYTFEDVPFHSSYAHSQSLDRLMNPLIDQYLYYLVRTQTTGTGGTQ
ILAFSQAGPSSMANQARNWVPGPSYRQQRVSTTKNQNNNSNFAWTGAAKFK
LNGRNSLMNPGVAMASHKDDEDRFFPSSGVLIFGKQGAGNDGMDYSQVLIT
DEEEIKATNPVATEEYGEVAINDQAANTQAQTGLVHNQGVIPGMVWQNRDV
YLQGPIWAKIPHTDGNFHPSPLMGGFGLKHPPPQILIKNTPVPADPPLTFN
QAKLNSFITQYSTGQVSVEIEWELQKENSKRWNPEIQYTSNYYKSTNVDFA
VNSDGVYSEPRPIGTRYLTRNL

The VP1 sequence of an AAV capsid isolated from crab-eating macaque (denoted as Bce.42) is set out as SEQ ID NO:43 (amino acids 1-736) and the locations of the associated variable regions and GBS and GH loop regions are defined in Table 5 below. The VP2 capsid protein spans amino acids 138-736 of SEQ ID NO:43 and the VP3 capsid protein spans amino acids 203-736 of SEQ ID NO:43.

(SEQ ID NO: 43)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQE
RLQEDTSFGGNLGRAVFQAKKRVLEPLGLVEEGAKTAPGKKRPVEQSPQEP
DSSSGIGKTGQQPAKKRLNFGQTGDSESVPDPQPLGEPPAAPSGLGPNTMA
SGGGAPMADNNEGADGVGNSSGNWHCDSTWLGDRVITTSTRTWALPTYNNH
LYKQISNSTSGGGTNDNTYFGYSTPWGYFDFNRFHCHFSPRDWQRLINNNW
GFRPKRLNFKLFNIQVKEVTTNEGTKTIANNLTSTVQVFTDSEYQLPYVLG
SAHQGCLPPFPADVFMVPQYGHLTLNNGSQALGRSSFYCLEYFPSQMLRTG
NNFQFSYTFEDVPFHSSYAHSQSLDRLMNPLIDQYLYYLVRTQTTGTGGTQ
TLAFSQAGPSSMANQARNWVPGPSYRQQRVSTTKDQNNNSNFAWTGAAKFK
LNGRNSLMNPGVAMASHKDDEDRFFPSSGVLIFGKQGAGNDGVDYSQVLIT
DEEEIKATNPVATEEYGEVAVNDQAANTQAQTGLVHNQGVIPGMVWQNRDV
YLQGPIWAKIPHTDGNFHPSPLMGGFGLKHPPPQILIKNTPVPADPPLTLN
QAKLNSFITQYSTGQVSVEIEWELQKENSKRWNPEIQYTSNYYKSTNVDFA
VNSDGVYSEPRPIGTRYLTRNL

The VP1 sequence of an AAV capsid isolated from crab-eating macaque (denoted as Bce.43) is set out as SEQ ID NO:44 (amino acids 1-730) and the locations of the associated variable regions and GBS and GH loop regions are defined in Table 5 below. The VP2 capsid protein spans amino acids 138-730 of SEQ ID NO:44 and the VP3 capsid protein spans amino acids 199-730 of SEQ ID NO:44.

(SEQ ID NO: 44)
MAADGYLPDWLEDNLSEGIREWWALKPGAPQPKANQQKQDDGRGLVLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVDSPDSTS
GIGKKGQQPARKRLNFGQTGDAESVPDPQPIGEPPAAPSGLGSGTMAAGGG
APMADNNEGADGVGNAPGNWHCDSTWLGNRVITTSTRTWALPTYNNHLYKQ
ISSSSSGATNDNHYFGYSTPWGYFDFNRFHCHFSPRDWPRLINNNWGFRPK
RLRFKLFNIQVKEVTTNDGVTTIANNLTSTVQVFSDSEYQLPYVLGSAHQG
CLPPFPADVFMIPQYGYLTLNNGSQSVGRSSFYCLEYFPSQMLRTGNNFEF
SYEFEDVPFHSSYAHSQSLDRLMNPLIDQYPYYLARTQSSTGTSTRELQFH
QAGPATMAEQSKNWLPGPCFRQQRISKTTDNNNNSNFAWTGATKYHLNGRN
SLTNPGVPMATHKDDESVFFPINGVLVFGKTGASNKTTLENVLMTDEEEIK
ATNPVATEEYGVVSSNIQSQNSNPATQTVNNQGALPGMVWQNRDVYLQGPI
WAKIPHTDGNFHPSPLMGGFGLKHPPPQILIKNTPVPANPPETFTPAKFAS
FITQYSTGQVSVEIEWELQKENSKRWNPEIQYTSNYDKQTGVDFAVDTQGV
YSEPRPIGTRYLTRNL

The VP1 sequence of an AAV capsid isolated from crab-eating macaque (denoted as Bce.44) is set out as SEQ ID NO:45 (amino acids 1-730) and the locations of the associated variable regions and GBS and GH loop regions are defined in Table 5 below. The VP2 capsid protein spans amino acids 138-730 of SEQ ID NO:45 and the VP3 capsid protein spans amino acids 199-730 of SEQ ID NO:45.

(SEQ ID NO: 45)
MAADGYLPDWLEDNLSEGIREWWALKPGAPQPKANQQKQDDGRGLMLPGYK
YLGPFNGLDKGEPVNEADAAALEHDKAYDQQLKAGDNPYLRYNHADAEFQE
RLQEDTSFGGNLGRAVFQAKKRVLEPLGLVEEAAKTAPGKKRPVDSPDSTS
GIGKKGQQPARKRLNFGQTGDAESVPDPQPIGEPPAAPSGLGSGTMAAGGG
APMADNNEGADGVGNASGSWHCDSTWLGNRVITTSTRTWALPTYNNHLYKQ
ISSSSSGATNDNHYFGYSTPWGYFDFNRFHCHFSPRDWQRLINNNWGFRPK
RLRFKLFNIQVKEVTTNDGVTTIANNLTSTVQVFSDSEYQLPYVLGSAHQG
CLPPFPADVFMIPQYGYLTLNNGRQSVGRSSFYCLEYFPSQMLRTGNNFEF
SYEFEDVPFHSSYAHSQSLDRLMNPLIDQYPYYLARTQSSTGTSTRELQFH
QAGPATMAEQSKNWLPGPCFRQQRISKTTDNNNNSNFAWTGATKYHLNGRN
SLTNPGVPMATHKDDESVFFPINGVLVFGKTGASNKTTLENVPMTDEEEIK
ATNPVATEEYGVVSSNIQSQNSNPTTQTVNNQGALPGMVWQNRDVYLQGPI
WAKIPHTDGNFHPSPLMGGFGLKHPPPQILIKNTPVPANPPETFTPAKFAS
FITQYSTGQVSVEIEWELQKENNKRWNPEIQYTSNYDKQTGVDFAVDTQGV
YSEPRPIGTRYLTRNL

The VP1 sequence of an AAV capsid isolated from crab-eating macaque (denoted as Bce.45) is set out as SEQ ID NO:46 (amino acids 1-730) and the locations of the associated variable regions and GBS and GH loop regions are defined in Table 5 below. The VP2 capsid protein spans amino acids 138-730 of SEQ ID NO:46 and the VP3 capsid protein spans amino acids 199-730 of SEQ ID NO:46.

(SEQ ID NO: 46)
MAADGYLPDWLEDNLSEGIREWWALKPGAPQPKANQQKQDDGRGLVLPGYK
YLGPFSGLDKGEPVNEADAAALEHDKAYDQQLKAGDNPYLRYNHADAEFQE
RLQEDTSFGGNLGRAVFQAKKRVLEPLGLVEEAAKTAPGKKRPVDSPDSTS
GIGKKGQQPARKRLNFGQTGDAESVPDPQPIGEPPAAPSGLGSGTMAAGGG
APMADNNEGADGVGNASGNWHCDSTWLGNRVITTSTRTWALPTYNDHLYKQ
ISSSSSGATNDNHYFGYSTPWGYFDFNRFHCHFSPRDWQRLINNNWGFRPK
RLRFKLFNIQVKEVTTNDGVTTIANNLTSTVQVFSDSEYQLPYVLGSAHQG
CLPPFPADVFMIPQYGYLTLNNGSQSVGRSSFYCLEYFPSQMLRTGNNFEF
SYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRN
SLTNPGVPMATHKDDESVFFPINGVLVFGKTGASNKTTLENVLMTDEEEIK
ATNPVATEEYGVVSSNIQSQNSNPTTQTVNNQGALPGMVWRNRDVYLQGPI
WAKIPHTDGNFHPSPLMGGFGLKHPPPQILIKNTPVPANPPETFTPAKFAS
FITQYSTGQVSVEIEWELQKENSKRWNPEIQYTSNYDKQTGVDFAVDTQGV
YSEPRPIGTRYLTRNL

The VP1 sequence of an AAV capsid isolated from crab-eating macaque (denoted as Bce.46) is set out as SEQ ID NO:47 (amino acids 1-730) and the locations of the associated variable regions and GBS and GH loop regions are defined in Table 5 below. The VP2 capsid protein spans amino acids 138-730 of SEQ ID NO:47 and the VP3 capsid protein spans amino acids 199-730 of SEQ ID NO:47.

(SEQ ID NO: 47)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEF
QERLQEDTSFGGNLGRAVFQAKKRVLEPLGLVEEAAKTAPGKKRPVDSPD
STSGIGKKGQQPARKRLNFGQTGDAESVPDPQPIGEPPAAPSGLGSGTMA
AGGGAPMADNNEGADGVGNASGNWHCDSTWLGNRVITTSTRTWALPTYNN
HLYKQISSSSSGATNDNHYEGYSTPWGYFDENREHCHFSPRDWQRLINNN
WGFRPKRLRFKLFNIQVKEVTTNDGVTTIANNLTSTVQVFSDSEYQLPYV
LGSAHQGCLPPFPADVFMIPQYGYLTLNNGSQSVGRSSFYCLEYFPSQML
RTGNNFEFSYEFEDVPFHSSYAHSQSLDRLMNPLIDQYLYYMARTQSSTG
TSTREPQFHQAGPATMAEQSKNWLPGPCFRQQRISKTTDNNNNSNFAWTG
ATKYHLNGRNSLTNPGVPMATHKDDESVFFPINGVLVFGKTGASNKTTLE
NVLMTDEEEIKATNPVATEEYGVVSSNIQSQNSNPTTQTVNNQGALPGMV
WQNRDVYLQGPIWAKIPHTDGDFHPSPLMGGFGLKHPPPQILIKNTPVPA
NPPETFTPAKFASFITQYSTGQVSVEIEWELQKENSKRWNPEIQYTSNYD
KQTGVDFAVDTQGVYSEPRPIGTRYLTRNL

The VP1 sequence of an AAV capsid isolated from cynomolgus macaque (denoted as Bcy.20) is set out as SEQ ID NO:48 (amino acids 1-730) and the locations of the associated variable regions and GBS and GH loop regions are defined in Table 5 below. The VP2 capsid protein spans amino acids 138-730 of SEQ ID NO:48 and the VP3 capsid protein spans amino acids 199-730 of SEQ ID NO:48.

(SEQ ID NO: 48)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEF
QERLQEDTSFGGNLGRAVFQAKKRVLEPLGLVEEAAKTAPGKKRPVDSPD
STSGIGKKGQQPARKRLNFGQTGDAESVPDPQPIGEPPAAPSGLGSGTMA
AGGGAPMADNNEGADGVGNASGNWHCDSTWLGNRVITTSTRTWALPTYNN
HLYKQISSGSSGATNDNHYEGYSTPWGYFDENREHCHFSPRDWQRLINNN
WGFRPKRLRFKLFNIQVKEVTTNDGVTTIANNLTSTVQVFSDSEYQLPYV
LGSAHQGCLPPFPADVFMIPQYGYLTLNNGSQSVGRSSFYCLEYFPSQVL
RTGNNFEFSYEFEDVPFHSSYAHSQSLDRLMNPLIDQYLYYLARTQSSTG
TSTRELQFHQAGPATMAEQSKNWLPGPCFRQQRISKTSDNNNNSNFAWTG
ATKYHLNGRNSLTNPGVPMATHKDDESVFFPINGVLVFGKTGASNKTTLE
NVLMTDEEEIKATNPVATEEYGVVSSNIQSQNSNPTTQTVNNQGALPGMV
WQNRDVYLQGPIWAKIPHTDGNFHPSPLMGGFGLKHPPPQILIKNTPVPA
NPPETFTPAKFASFITQYSTGQVSVEIEWELQKENSKRWNPEIQYTSNYD
KQTGVDFAVDTQGVYSEPRPIGTRYLTRNL

The VP1 sequence of an AAV capsid isolated from cynomolgus macaque (denoted as Bcy.22) is set out as SEQ ID NO:49 (amino acids 1-730) and the locations of the associated variable regions and GBS and GH loop regions are defined in Table 5 below. The VP2 capsid protein spans amino acids 138-730 of SEQ ID NO:49 and the VP3 capsid protein spans amino acids 199-730 of SEQ ID NO:49.

(SEQ ID NO: 49)
MAADGYLPDWLEDNLSEGIREWWALKPGAPQPKANQQKQDDGRGLVLPGY
RYLGPFNGLDKGEPVNEADAAALEHDKAYDQQLKAGDNPYLRYNHADAEF
QERLQEDTSFGGNLGRAVFQAKKRILEPLGLVEEAAKTAPGKERPVDSPD
STSGIGKKGQQPARKRLNFGQTGDAESVPDPQPIGEPPAAPSGLGSGTMA
AGGGAPMADNNEGADGVGNASGNWHCDSTWLGNRVITTSTRTWALPTYDN
HLYKQISSSSSGATNDNHYEGYSTPWGYFDENREHCHFSPRDWQRLINNN
WGFRPKRLRFKLFNIQVKEVTTNDGVTTIANNLTSTVQVFSDSEYQLPYV
LGSAHQGCLPPFPADVFMIPQYGYLTLNNGSQSVGRSSFYCLEYFPSQML
RTGNNFEFSYEFEDVPFHSSYAHSQSLDRLMNPLIDQYLYYLARTQSSTG
TSTRELQFHQAGPATMAEQSKNWLPGPCFRQQRISKTTDNNNNSNFAWTG
ATKYHLNGRNSLTNPGVPMATHKDDESVFFPINGVLVLGKTGASNKTTLE
NVLMTDEEEIKATNPVATEEYGVVSSNIQSQNSNPTTQTVNNQGALPGMV
WQNRDVYLQGPIWAKIPHTDGNFHPSPLMGGFGLKHPPPQILIKNTPVPA
NPPETFTPAKFASFITQYSTGQVSVEIEWELQKENSKRWNPEIQYTSNYD
KQTGVDFAVDTQGVYSEPRPIGTRYLTRNL

The VP1 sequence of an AAV capsid isolated from cynomolgus macaque (denoted as Bcy.23) is set out as SEQ ID NO:50 (amino acids 1-730) and the locations of the associated variable regions and GBS and GH loop regions are defined in Table 5 below. The VP2 capsid protein spans amino acids 138-730 of SEQ ID NO:50 and the VP3 capsid protein spans amino acids 199-730 of SEQ ID NO:50.

(SEQ ID NO: 50)
MAADGYLPDWLEDNLSEGIREWWALKPGAPRPKANQQKQDDGRGLVLPGY
KYLGPFNGLDKGEPVNEADAAALEHDKAYDQQLKAGDNPYLRYNHADAEF
QERLQEDTSFGGNLGRAVFQAKKRVLEPLGLVEEAAKTAPGKKRPVDSPD
STSGIGKKGQQPARKRLNFGQTGDAESVPDPQPIGEPPAAPSGLGSGTMA
AGGGAPMADNNEGADGVGNASGNWHCDSTWLGNRVITTSTRTWALPTYNN
HLYKQISSSSSGATNDNHYEGYSTPWGYFDENREHCHFSPRDWQRLINNN
WGFRPKRLRFKLFNIQVKEVTTNDGVTTIANNLTSTVQVFSDSEYQLPYV
LGSAHQGCLPPFPADVFMIPQYGYLTLNNGSQSVGRSSFYRLEYFPSQML
RTGNNFEFSYEFEDVPFHSSYAHSQSLDRLMNPLIDQYLYYLARTQSSTG
TSTRELQFHQAGPATMAEQSKNWLPGPCFRQQRISKTTDNNNNSNFAWTG
ATKYHLNGRNSLTNPGVPMATHRDDESVFFPINGVLVFGKTGASNKTTLE
NVLMTDEEEIKATNPVATEEYGVVSSNIQSQNSNPTTQTVNNQGALPGMV
WQNRDVYLQGPIWAKIPHTDGNFHPSPLMGGFGLKHPPPQILIKNTPVPA
NPPETFTPAKFASFITQYSTGQVSVEIEWELQKENSKRWNPEIQYTSNYD
KQTGVDFAVDTQGVYSEPRPIGTRYLTRNL

The VP1 sequence of an AAV capsid isolated from marmoset (denoted as Bma.42) is set out as SEQ ID NO:51 (amino acids 1-736) and the locations of the associated variable regions and GBS and GH loop regions are defined in Table 5 below. The VP2 capsid protein spans amino acids 138-736 of SEQ ID NO:51 and the VP3 capsid protein spans amino acids 203-736 of SEQ ID NO:51.

(SEQ ID NO: 51)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDTESVPDPQPLREPPAAPSGLGP
NTMASGGGAPMADNNEGADGVGNSSGNWHCDSTWLGDRVITTSTRTWALP
TYNNHLYKQISNGTSGGSTNDNTYEGYSTPWGYFDENREHCHFSPRDWQR
LINNNWGFRPKRLNEKPFNIQVKEVTTNEGTKTIANNPTSTVQVFTDSEY
QLPYVLGSARQGCLPPFPADVFMVPQYGYLTLNNGSQALGRSSFYCLEYF
PSQMLRTGDNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDEDRFFPSSGVLIFGKQGAGN
DGVDYSQVLITDEEEIKATNPVATEEYGAVAINNQAANTLAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

The VP1 sequence of an AAV capsid isolated from marmoset (denoted as Bma.43) is set out as SEQ ID NO:52 (amino acids 1-736) and the locations of the associated variable regions and GBS and GH loop regions are defined in Table 5 below. The VP2 capsid protein spans amino acids 138-736 of SEQ ID NO:52 and the VP3 capsid protein spans amino acids 203-736 of SEQ ID NO:52.

(SEQ ID NO: 52)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGANTAPGKKRPVEQSP
QEPDSSSGIGKTGQQPAKKRLNFGQTGDSESVPDPQPLREPPAAPSGLGP
NTMASGGGAPMADNNEGADGVGNSSGNWHCDSTWLGDRVITTSTRTWALP
TYNNHLYKQISNGTSGGSTNDNTYEGYSTPWGYFDENREHCHFSPRDWQR
LINSNWGFRPKRLNFKLFNIQVKEVTTNEGTKTIANNLTSTVQVFTDSEY
QLPYVLGSAHQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDEDRFFPSSGVLIFGKQGAGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

The VP1 sequence of an AAV capsid isolated from pig (denoted as Bpo.1) is set out as SEQ ID NO:53 (amino acids 1-716) and the locations of the associated variable regions and GBS and GH loop regions are defined in Table 5 below. The VP2 capsid protein spans amino acids 137-716 of SEQ ID NO:53 and the VP3 capsid protein spans amino acids 184-716 of SEQ ID NO:53.

(SEQ ID NO: 53)
MSFVDHPPDWLEEIGEGLKEFLGLEPGPPKPKPNQQKQDNARGLVLPGYN
YLGPGNGLDRGEPVNRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYRFVSTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKEDSKRWNPEIQYTNNYNNPEFVDFAPDTSGE
YRTTRAIGTRYLTRPL

The VP1 sequence of an AAV capsid isolated from pig (denoted as Bpo.2) is set out as SEQ ID NO:54 (amino acids 1-716) and the locations of the associated variable regions and GBS and GH loop regions are defined in Table 5 below. The VP2 capsid protein spans amino acids 137-716 of SEQ ID NO:54 and the VP3 capsid protein spans amino acids 184-716 of SEQ ID NO:54.

(SEQ ID NO: 54)
MSFVDHPPDWLEEIGEGLKEFLGLEPGPPKPKPNQQKQDNARGLVLPGYN
YLGPGNGLDRGEPVNRADEVAREHDISYNEQLQAGDNPYLKYNHADAEFQ
EKLADDTSFGGNLGKAVFQAKKRALEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYREVGTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.3) is set out as SEQ ID NO:55 (amino acids 1-716) and the locations of the associated variable regions and GBS and GH loop regions are defined in Table 5 below. The VP2 capsid protein spans amino acids 137-716 of SEQ ID NO:55 and the VP3 capsid protein spans amino acids 184-716 of SEQ ID NO:55.

(SEQ ID NO: 55)
MSFVDHPPDWFEEIGEGLKEFLGLEPGPPKPKPNQQKQDNARGLVLPGYN
YLGPGNGLDRGEPANRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFG
EVPLHCSFAPSQNLFKLANPLVDQHLYRFVSTDTSGNIQFQKNLKARYAN
TYKNWFPGPMCRTQGWYTGSGTYNRPGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLTKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.4) is set out as SEQ ID NO:56 (amino acids 1-716) and the locations of the associated variable regions and GBS and GH loop regions are defined in Table 5 below. The VP2 capsid protein spans amino acids 137-716 of SEQ ID NO:56 and the VP3 capsid protein spans amino acids 184-716 of SEQ ID NO:56.

(SEQ ID NO: 56)
MSFVDHPPDWLEEIGEGLKEFLGLEPGPPKPKPNQQKQDNARGLVLPGYN
YLGPGNGLDRGEPVNRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYRFVSTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.6) is set out as SEQ ID NO:57 (amino acids 1-716) and the locations of the associated variable regions and GBS and GH loop regions are defined in Table 5 below. The VP2 capsid protein spans amino acids 137-716 of SEQ ID NO:57 and the VP3 capsid protein spans amino acids 184-716 of SEQ ID NO:57.

(SEQ ID NO: 57)
MSFVDHPPDWLEEIGEGLKEFLGLEPGPPKPKPNQQKQDNARGLVLPGYN
YLGPGNGLDRGEPVNRADEVAREHGISYNEQLQAGDDPYLKYNHADAEFQ
EKLADDTSFGGNLGKAVFQAKKRVLEPFGLVEEPAKTAAKGERIDDHYPK
KKKARVEETEAGTGGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSLEFTYSFE
EVPFHCSFAPSQNLFKLANPLVDQYLYRFVSTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.8) is set out as SEQ ID NO:58 (amino acids 1-716) and the locations of the associated variable regions and GBS and GH loop regions are defined in Table 5 below. The VP2 capsid protein spans amino acids 137-716 of SEQ ID NO:58 and the VP3 capsid protein spans amino acids 184-716 of SEQ ID NO:58.

(SEQ ID NO: 58)
MSFVDHPPDWLEEIGEGLKEFLGLKPGPPKPKPNQQKQDNARGLVLPGYN
YLGPGNGLGRGEPVNRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYRFVSTDTSGNIQFQKNLKARYAN
TYKNWFPGPMCRTQGWYTGSGTYNRSGVTNFATSNRMDLEGASYQANPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSL
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.13) is set out as SEQ ID NO:59 (amino acids 1-716) and the locations of the associated variable regions and GBS and GH loop regions are defined in Table 5 below. The VP2 capsid protein spans amino acids 137-716 of SEQ ID NO:59 and the VP3 capsid protein spans amino acids 184-716 of SEQ ID NO:59.

(SEQ ID NO: 59)
MSFVDHPPDWLEEIGEGLKEFLGLEPGPPKPKPNQQKQDNARGLVLPGYN
YLGPGNGLDRGEPVNRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLASTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYRFVSTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDAPDTSGEY
RTTRAIGTRYLTRPL

The VP1 sequence of an AAV capsid isolated from pig (denoted as Bpo.18) is set out as SEQ ID NO:60 (amino acids 1-716) and the locations of the associated variable regions and GBS and GH loop regions are defined in Table 5 below. The VP2 capsid protein spans amino acids 137-716 of SEQ ID NO:60 and the VP3 capsid protein spans amino acids 184-716 of SEQ ID NO:60.

(SEQ ID NO: 60)
MSFVDHPPDWLEEIGEGLKEFLGLEPGPPKPKPNQQKQDNARGLVLPGYN
YLGPGNGLDRGEPVNRADEVAREHDISYNEQLQAGDDPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWSPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
EVPFHCSFAPSQNLFKLANPLVDQYLYRFVSTDTSGNIQFQKNLKARYAN
TYKNWFPGPMRRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.20) is set out as SEQ ID NO:61 (amino acids 1-716) and the locations of the associated variable regions and GBS and GH loop regions are defined in Table 5 below. The VP2 capsid protein spans amino acids 137-716 of SEQ ID NO:61 and the VP3 capsid protein spans amino acids 184-716 of SEQ ID NO:61.

(SEQ ID NO: 61)
MSFVDHPPDWLEEIGEGLKEFLGLEPGPPKPKPNQQKQDNARGLVLPGYN
YLGPGNGLDRGEPVNRADEVAREHDISYNEQLQAGDNPYLKYNHADAEFQ
EKLADDTSFGGNLGKAVFQAKKRVLEPFGLVEEPAKTAAKGERIDDHYPK
KKKARVEETEAGTSGGQQLQIPAQPASSLGADTMSAGGGSPLGDNNQGAD
GVGNASGDWHCDSTWMGDRVITKSTRTWVLPSYNNHLYKEIHSGSVDGSN
ANAYEGYSTPWGYFDENREHSHWGPRDWQRLVNNYWGFRPRSLKVKIFNI
QVKEVTVQDATTTIANNLTSTVQVFTDDDYQLPYVIGNGTEGCLPAFPPQ
VFTLPQYGYATLNRNNTDDPTERSSFECLEYEPSKMLRTGNSFEFTYSFE
GVPFHCSFAPSQNLFKLANPLVDQYLYREVSTDTSGNIQFQKNLKARYAN
TYKNWFPGPMCRTQGWYTGSGTYNRSGVTNFATSNRMDLEGASYQVNPQP
NGMTNTLQDSNKYALENTMIFNSQNAEPGTTSLYQENNLLITSESETQPV
NRVAYDTGGQMATNAQSTNLAPTVGTYNHQEMLPGSVWMDRDVYLQGPIW
AKIPETGAHFHPSPAMGGFGLKHPPPMMLIKNTPVPSNVTAFSEIPVKSF
ITQYSTGQVTVEMEWELKKENSKRWNPEIQYTNNYNNPEFVDFAPDTSGE
YRTTRAIGTRYLTRPL

The VP1 sequence of an AAV capsid isolated from pig (denoted as Bpo.23) is set out as SEQ ID NO:62 (amino acids 1-716) and the locations of the associated variable regions and GBS and GH loop regions are defined in Table 5 below. The VP2 capsid protein spans amino acids 137-716 of SEQ ID NO:62 and the VP3 capsid protein spans amino acids 184-716 of SEQ ID NO:62.

(SEQ ID NO: 62)
MSFVDHPPDWLEEIGEGLKEFLGLEPGPPKPKPNQQKRDNARGLVLPGY
NYLGPGNGLDRGEPVNRVDEVARERDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYEGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPLQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.24) is set out as SEQ ID NO:63 (amino acids 1-716) and the locations of the associated variable regions and GBS and GH loop regions are defined in Table 5 below. The VP2 capsid protein spans amino acids 137-716 of SEQ ID NO:63 and the VP3 capsid protein spans amino acids 184-716 of SEQ ID NO:63.

(SEQ ID NO: 63)
MSFVDHPPDWLEEIGEGLKEFLGLEPGPPKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGECRTTRAIGTRYLTRPL

The VP1 sequence of an AAV capsid isolated from pig (denoted as Bpo.27) is set out as SEQ ID NO:64 (amino acids 1-716) and the locations of the associated variable regions and GBS and GH loop regions are defined in Table 5 below. The VP2 capsid protein spans amino acids 137-716 of SEQ ID NO:64 and the VP3 capsid protein spans amino acids 184-716 of SEQ ID NO:64.

(SEQ ID NO: 64)
MSFVDHPPDWLEEIGEGLKEFLGLEPGPPKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVDNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMVFNSQNAEPGTTSLYQENNL
LITSESETQPVNRVAYNTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.28) is set out as SEQ ID NO:65 (amino acids 1-716) and the locations of the associated variable regions and GBS and GH loop regions are defined in Table 5 below. The VP2 capsid protein spans amino acids 137-716 of SEQ ID NO:65 and the VP3 capsid protein spans amino acids 184-716 of SEQ ID NO:65.

(SEQ ID NO: 65)
MSFVDHPPDWLEEIGEGLKEFLGLEPGPPKPKPNQQKRDNARGLVLPGY
NYLGPGNGLGRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGLRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLADQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMISNSQNAEPGTTSLYRENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.29) is set out as SEQ ID NO:66 (amino acids 1-716) and the locations of the associated variable regions and GBS and GH loop regions are defined in Table 5 below. The VP2 capsid protein spans amino acids 137-716 of SEQ ID NO:66 and the VP3 capsid protein spans amino acids 184-716 of SEQ ID NO:66.

(SEQ ID NO: 66)
MSFVDHPPDWLEEIGEGLKEFLGLEPGPPKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESGTQPVNRVAYDTGGQMATNAQSTNLAPTVGTYNHQEMLPGSVW
MDRGVYLQGPIWAKIPETGAHFHPSPAMGGFGLKHPPPMMLIKNTPVPS
NVTAFSEIPVKSFVTQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.33) is set out as SEQ ID NO:67 (amino acids 1-716) and the locations of the associated variable regions and GBS and GH loop regions are defined in Table 5 below. The VP2 capsid protein spans amino acids 137-716 of SEQ ID NO:67 and the VP3 capsid protein spans amino acids 184-716 of SEQ ID NO:67.

(SEQ ID NO: 67)
MSFVDHPPDWLEEIGEGLKEFLGLEPGPPKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNAHAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.35) is set out as SEQ ID NO:68 (amino acids 1-716) and the locations of the associated variable regions and GBS and GH loop regions are defined in Table 5 below. The VP2 capsid protein spans amino acids 137-716 of SEQ ID NO:68 and the VP3 capsid protein spans amino acids 184-716 of SEQ ID NO:68.

(SEQ ID NO: 68)
MSFVDHPPDWLEEIGEGLKEFLGLEPGPPKPKPNQQKRDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVGFAPDTSGEYRTTRAIGTRYLTRPL

The VP1 sequence of an AAV capsid isolated from pig (denoted as Bpo.36) is set out as SEQ ID NO:69 (amino acids 1-716) and the locations of the associated variable regions and GBS and GH loop regions are defined in Table 5 below. The VP2 capsid protein spans amino acids 137-716 of SEQ ID NO:69 and the VP3 capsid protein spans amino acids 184-716 of SEQ ID NO:69.

(SEQ ID NO: 69)
MSFVDHPPDWLEEIGEGLKEFLGLEPGPPKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIGDH
YPKKKKARVEETEAGTSGGQQPQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYLPSKMLRTGNS
FEFTYSFEEVPFHCSFAPSQNLFKLANPLVDQYLYRFVSTDTSGNIQFQ
KNLKARYANTYKNWFPGPMCRTQGWYTGSGTYNRSGVTNFATSNRMDLE
GASYQVNPQPNGMTNTLQDSNKYALENTMIFNSQNAEPGTTSLYQENNL
LITSESETQPVNRVAYNTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSFITQYSTGQVTVEMEWELKKENSKRWNPEIQYTNNYN
NPEFVDFAPDTSGEYRTTRAIGTRYLTRPL

The VP1 sequence of an AAV capsid isolated from pig (denoted as Bpo.37) is set out as SEQ ID NO:70 and (amino acids 1-716) and the locations of the associated variable regions and GBS and GH loop regions are defined in Table 5 below. The VP2 capsid protein spans amino acids 137-716 of SEQ ID NO:70 and the VP3 capsid protein spans amino acids 184-716 of SEQ ID NO:70.

(SEQ ID NO: 70)
MSFVDHPPDWLEEIGEGLKEFLGLKPGPLKPKPNQQKQDNARGLVLPGY
NYLGPGNGLDRGEPVNRADEVAREHDISYNEQLQAGDNPYLKYNHADAE
FQEKLADDTSFGGNLGKAVFQAKKRVLEPFGLVEEPAKTAAKGERIDDH
YPKKKKARVEETEAGTSGGQQLQIPAQPASSLGADTMSAGGGSPLGDNN
QGADGVGNASGDWHCDSTWMGDRVITKSTRTWVLPSYNNHLYKEIHSGS
VDGSNANAYFGYSTPWGYFDFNRFHSHWSPRDWQRLVNNYWGFRPRSLK
VKIFNIQVKEVTVQDATTTIANNLTSTVQVFTDDDYQLPYVIGNGTEGC
LPAFPPQVFTLPQYGYATLNRNNTDDPTERSSFFCLEYFPSKMLRTGNS
FEFTYSFEEVPFHCSFAPSQNLFKLANPLVDQYLYRFVSTDTPGNIQFQ
KNLKARYANTYKNWFPGPMCRTQGWYTGSGTYNRSGVTNFATSNRMDLE
GASYQANPQPNGMTNTLQDSNKYALENTMIFNSQNAEPGTTSLYQENNL
LITSESETQPVNRVAYDTGGQMATNAQSTNLAPTVGTYNHQEMLPGSVW
MDRDVYLQGPIWAKIPETGAHFHPSPAMGGFGLKHPPPMMLIKNTPVPS
NVTAFSEIPVKSLITQYSTGQVTVEMEWELKKENSKRWNPEIQYTNNYN
NPEFVDFAPDTSGEYRTTRAIGTRYLTRPL

The VP1 sequence of an AAV capsid isolated from rhesus macaque (denoted as Brh.26) is set out as SEQ ID NO:71 and (amino acids 1-736) and the locations of the associated variable regions and GBS and GH loop regions are defined in Table 5 below. The VP2 capsid protein spans amino acids 138-736 of SEQ ID NO:71 and the VP3 capsid protein spans amino acids 203-736 of SEQ ID NO:71.

(SEQ ID NO: 71)
MAADGYLPDWLEDNLSEGIREWWDLKPGAPKPKANQQKQDDGRGLVLPG
YKYLGPFNGLDKGEPANAADAAALEHDKAYDQQLKAGDNPYLRYNHADA
EFQERLQEDTSFGGNLGRAIFQAKKRVLEPLGLVEEGAKTAPGKKGPVE
QSPQEPDSSSGIGKTGQQPAKKRLNFGQTGDSESVPDPQPLGEPPAAPS
GLGPNTMASGGGAPMADNNEGADGVGNSSGNWHCDSTWLGDRVITTSTR
TWALPTYNNHLYKQISNGTSGGSTNDNTYFGYSTPWGYFDFNRFHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTGNNFQFSYTFEDVPFHSSYAHSQSLDRLMNPLID
QYLYYLVRTQTTGTGGTQTLAFSQAGPSSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYLQGPIWAKIPHTDGNFHPSPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.27) is set out as SEQ ID NO:72 and (amino acids 1-736) and the locations of the associated variable regions and GBS and GH loop regions are defined in Table 5 below. The VP2 capsid protein spans amino acids 138-736 of SEQ ID NO:72 and the VP3 capsid protein spans amino acids 203-736 of SEQ ID NO:72.

(SEQ ID NO: 72)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAIFQAKKRVLEPLGLVEEGAKTAPGKKRPVE
QSPQEPDSSSGIGKTGQQPAKKRLNFGQTGDSESVPDPQPLGEPPAAPS
GLGPNTMASGGGAPMADNNEGADGVGNPSGNWHCDSTRLGDRVITTSTR
TWALPTYNNHLYKQISNGTSGGSTNDNTYEGYSTPWGYFDENREHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTSNNFQFSYTFEDVPFHSSYAHSQSLDRLMNPLID
QYLYYLVRTQTTGTGGTQTLAFSQAGPSSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYLQGPIWAKIPHTDGNFHPSPLMGG
FGLKLPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.28) is set out as SEQ ID NO:73 and (amino acids 1-736) and the locations of the associated variable regions and GBS and GH loop regions are defined in Table 5 below. The VP2 capsid protein spans amino acids 138-736 of SEQ ID NO:73 and the VP3 capsid protein spans amino acids 203-736 of SEQ ID NO:73.

(SEQ ID NO: 73)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAIFQAKKRVLEPLGLVEEGAKTAPGKKRPVE
QSPQEPDSPSGIGKTGQQPAKKRLNFGQTGDSESVPDPQPLGEPPAAPS
GLGPNTMASGGGAPMADNNEGADGVGNSSGNWHCDSTWLGDRVITTSTR
TWALPTYNNHLYKQISNGTSGGSTNDNTYFGYSTPWGYFDFNRFHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTGNNFQFSYTFEDVPFHSSYAHSQSLDRLMNPLID
QYLYYLVRTQTTGTGGTQTLAFSQAGPSSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYLQGPIWAKIPHTDGNFHPSPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.29) is set out as SEQ ID NO:74 and (amino acids 1-736) and the locations of the associated variable regions and GBS and GH loop regions are defined in Table 5 below. The VP2 capsid protein spans amino acids 138-736 of SEQ ID NO:74 and the VP3 capsid protein spans amino acids 203-736 of SEQ ID NO:74.

(SEQ ID NO: 74)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAIFQAKRRVLEPLGLVEEGAKTAPGKKRPVE
QSPQEPDSSSGIGKTGQQPAKKRLNFGQTGDSESVPDPQPLGEPPAAPS
GLGPNTMASGGGAPMADNNEGADGVGNSSGNWHCDSTWLGDRVITTSTR
TWALPTYNNHLYKQISNGTSGGSTNDNTYFGYSTPWGYFDFNRFHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTSNNFQFSYTFEDVPFHSSCAHSQSLDRLMNPLID
QYLYYLVRTQTTGTGGTQTLAFSQAGPGSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYLQGPIWAKIPHTDGNFHPPPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.30) is set out as SEQ ID NO:75 and (amino acids 1-736) and the locations of the associated variable regions and GBS and GH loop regions are defined in Table 5 below. The VP2 capsid protein spans amino acids 138-736 of SEQ ID NO:75 and the VP3 capsid protein spans amino acids 203-736 of SEQ ID NO:75.

(SEQ ID NO: 75)
MAADGYLPDWLEDNLSEGIREWWDLKPGAPKPKANQQKQDDGRGLVFPG
YKYLGPFNGLDKGEPVNAADAAALEHDKAYDQQLKAGDNPYLRYNHADA
EFQERLQEDTSFGGNLGRAIFQAKKRVLEPLGLVEEGAKTAPGKKRPVE
QSPQEPDSSSGIGKTGRQPAKKRLNFGQTGDSESVPDPQPLGEPPAAPS
GLGPNTMASGGGAPMADNNEGADGVGNSSGNWHCDSTWLGDRVITTSTR
TWALPTYNNHLYKQISNGTPGGSTNDNTYFGYSAPWGYFDFNRFHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTGNNFQFSYTFEDVPFHSSYAHSQSLDRLMNPLID
QYLYYLVRTQTTGTGGTQTLAFSQAGPSSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYQQGPIWAKIPHTDGNFHPSPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.31) is set out as SEQ ID NO:76 and (amino acids 1-736) and the locations of the associated variable regions and GBS and GH loop regions are defined in Table 5 below. The VP2 capsid protein spans amino acids 138-736 of SEQ ID NO:76 and the VP3 capsid protein spans amino acids 203-736 of SEQ ID NO:76.

(SEQ ID NO: 76)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAIFQAKKRVLEPLGLVEEGAKTAPGKKRPVE
QSPQEPDSSSGIGKTGQQPAKKRLNSGQTGDSESVPDPQPLGEPPAAPS
GLGPNTMASGGGAPMADNNEGADGVGNSSGNWHCDSTWLGDRVITTSTR
TWALPTYNNHLYKQISNGTSGGSTNDNTYFGYSTPWGYFDFNRFHCHFS
PRDWQRLINNNWGFRPKRLNFKLFNIQVKEVTTNEGTKTIANNLTSTVQ
VFTDSEYQLPYVLGSAYQGCLPPFPADVFMVPQYGYLTLNNGSQALGRS
SFYCLEYFPSQMLRTGNNFQSSYTFEDVPFHSSYAHSQSLDRLMNPLID
QYLYYLVRTQTTGTGGTQTLAFSQAGPSSMANQARNWVPGPCYRQQRVS
TTTNQNNNSNFAWTGAAKFKLNGRDSLMNPGVAMASHKDDDDRFFPSSG
VLIFGKQGTGNDGVDYSQVLITDEEEIKATNPVATEEYGAVAINNQAAN
TQAQTGLVHNQGVIPGMVWQNRDVYLQGPIWAKIPHTDGNFHPSPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L

The VP1 sequence of an AAV capsid isolated from rhesus macaque (denoted as Brh.32) is set out as SEQ ID NO:77 and (amino acids 1-736) and the locations of the associated variable regions and GBS and GH loop regions are defined in Table 5 below. The VP2 capsid protein spans amino acids 138-736 of SEQ ID NO:77 and the VP3 capsid protein spans amino acids 203-736 of SEQ ID NO:77.

(SEQ ID NO: 77)
MAADGYLPDWLEDNLSEGIREWWDLKPGASKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTVPGKKRPVEQSP
QEPDSSSGIGKTGQQPAKKRLNFGQTGDSESVPDPQPLGEPPAAPSGLGP
NTMASGGGAPMADNNEGADGVGNSSGNWHCDSTWLGDRVITTSTRTWALP
TYNNHLYKQISNGTSGGSTNDNTYEGYSTPWGYFDENREHCHFSPRDWQR
LINNNWGFRPKRLNFKLFNIQVKEVTTNEGTKTIANNLTSTVQVFTDSEY
QLPYVLGSAYQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDDDRFFPSSGVLIFGKQGTGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

The VP1 sequence of an AAV capsid isolated from rhesus macaque (denoted as Brh.33) is set out as SEQ ID NO:78 and (amino acids 1-736) and the locations of the associated variable regions and GBS and GH loop regions are defined in Table 5 below. The VP2 capsid protein spans amino acids 138-736 of SEQ ID NO:78 and the VP3 capsid protein spans amino acids 203-736 of SEQ ID NO:78.

(SEQ ID NO: 78)
MAADGYLPDWLEDNLSEGIREWWDLKPGASKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTVPGKKRPVEQSP
QEPDSSSGIGKTGQQPAKKRLNFGQTGDSESVPDPQPLGEPPAAPSGLGP
NTMASGGGAPMADNNEGADGVGNSSGNWHCDSTWLGDRVITTSTRTWALP
TYNNHLYKQISNGTSGGSTNDNTYEGYSTPWGYFDENREHCHFSPRDWQR
LINNNWGFRPKRLNFKLFNIQVKEVTTNEGTKTIANNLTSTVQVFTDSEY
QLPYVLGSAYQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDDDRFFPSSGVLIFGKQGTGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

The VP1 sequence of an AAV capsid isolated from formosan macaque (denoted as Bfm.17) is set out as SEQ ID NO:79 and (amino acids 1-737) and the locations of the associated variable regions and GBS and GH loop regions are defined in Table 5 below. The VP2 capsid protein spans amino acids 138-736 of SEQ ID NO:79 and the VP3 capsid protein spans amino acids 203-736 of SEQ ID NO:79.

(SEQ ID NO: 79)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTRLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLSYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
EALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVDTEGVYSEHRPIGTRYLTRNL

The VP1 sequence of an AAV capsid isolated from formosan macaque (denoted as Bfm.18) is set out as SEQ ID NO:80 and (amino acids 1-737) and the locations of the associated variable regions and GBS and GH loop regions are defined in Table 5 below. The VP2 capsid protein spans amino acids 138-736 of SEQ ID NO:80 and the VP3 capsid protein spans amino acids 203-736 of SEQ ID NO:80.

(SEQ ID NO: 80)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLPYVPGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYPQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTEGVYSEPRPIGTRYLTRNL

The VP1 sequence of an AAV capsid isolated from formosan macaque (denoted as Bfm.20) is set out as SEQ ID NO:81 and (amino acids 1-737) and the locations of the associated variable regions and GBS and GH loop regions are defined in Table 5 below. The VP2 capsid protein spans amino acids 138-736 of SEQ ID NO:81 and the VP3 capsid protein spans amino acids 203-736 of SEQ ID NO:81.

(SEQ ID NO: 81)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPPGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSFFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIERELQKENSKRWNPEI
QYTSNYYKSTNVDFAVNTEGVYSEPRPIGTRYLTRNL

The VP1 sequence of an AAV capsid isolated from formosan macaque (denoted as Bfm.21) is set out as SEQ ID NO:82 and (amino acids 1-737) and the locations of the associated variable regions and GBS and GH loop regions are defined in Table 5 below. The VP2 capsid protein spans amino acids 138-736 of SEQ ID NO:82 and the VP3 capsid protein spans amino acids 203-736 of SEQ ID NO:82.

(SEQ ID NO: 82)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKERPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYEGYSTPWGYFDENREHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSAVLMFGKQGAG
KDNVDYSSVMLTSEEEIKTTNPVTTEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTESVYSEPRPIGTRYLTRNL

The VP1 sequence of an AAV capsid isolated from formosan macaque (denoted as Bfm.24) is set out as SEQ ID NO:83 and (amino acids 1-737) and the locations of the associated variable regions and GBS and GH loop regions are defined in Table 5 below. The VP2 capsid protein spans amino acids 138-736 of SEQ ID NO:83 and the VP3 capsid protein spans amino acids 203-736 of SEQ ID NO:83.

(SEQ ID NO: 83)
MAADGYLPDWLEDNLSEGIREWWALKPGAPKPKANQQKQDDGRGLVLPGY
KYLGPFNGPDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRSHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIRTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTEGVYSEPRPIGTRYLTRNL

The VP1 sequence of an AAV capsid isolated from formosan macaque (denoted as Bfm.25) is set out as SEQ ID NO:84 and (amino acids 1-737) and the locations of the associated variable regions and GBS and GH loop regions are defined in Table 5 below. The VP2 capsid protein spans amino acids 138-736 of SEQ ID NO:84 and the VP3 capsid protein spans amino acids 203-736 of SEQ ID NO:84.

(SEQ ID NO: 84)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRLFPSSGVLMFGKQGAG
KDNVDYSSVMLTSEEEVKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTEGVYSEPRPIGTRYLTRNL

The VP1 sequence of an AAV capsid isolated from formosan macaque (denoted as Bfm.27) is set out as SEQ ID NO:85 and (amino acids 1-737) and the locations of the associated variable regions and GBS and GH loop regions are defined in Table 5 below. The VP2 capsid protein spans amino acids 138-736 of SEQ ID NO:85 and the VP3 capsid protein spans amino acids 203-736 of SEQ ID NO:85.

(SEQ ID NO: 85)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKRRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYEGYSTPWGYFDENREHCHFSPRDWQR
LINNNWGFRPKKLSFKLFNIQVKEVTQNEGTKTIANNLTSTIQVFTDSEY
QLPYVLGSARQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVDTEGVYSEPRPIGTRYLTRNL

The VP1 sequence of an AAV capsid isolated from formosan macaque (denoted as Bfm.32) is set out as SEQ ID NO:86 and (amino acids 1-737) and the locations of the associated variable regions and GBS and GH loop regions are defined in Table 5 below. The VP2 capsid protein spans amino acids 138-736 of SEQ ID NO:86 and the VP3 capsid protein spans amino acids 203-736 of SEQ ID NO:86.

(SEQ ID NO: 86)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFDIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTEGVYSEPRPIGTRYLTRNL

The VP1 sequence of an AAV capsid isolated from formosan macaque (denoted as Bfm.33) is set out as SEQ ID NO:87 and (amino acids 1-737) and the locations of the associated variable regions and GBS and GH loop regions are defined in Table 5 below. The VP2 capsid protein spans amino acids 138-736 of SEQ ID NO:87 and the VP3 capsid protein spans amino acids 203-736 of SEQ ID NO:87.

(SEQ ID NO: 87)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSAVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTESVYSEPRPIGTRYLTRNL

The VP1 sequence of an AAV capsid isolated from formosan macaque (denoted as Bfm.34) is set out as SEQ ID NO:88 and (amino acids 1-737) and the locations of the associated variable regions and GBS and GH loop regions are defined in Table 5 below. The VP2 capsid protein spans amino acids 138-736 of SEQ ID NO:88 and the VP3 capsid protein spans amino acids 203-736 of SEQ ID NO:88.

(SEQ ID NO: 88)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDNTYFGYSTPWGYFDFNRFHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFSFSYTFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVYPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELQKENSKRWNPEI
QYTSNYYKSTNVDFAVNTEGVYSEPRPIGTRYLTRNL

The VP1 sequence of an AAV capsid isolated from formosan macaque (denoted as Bfm.35) is set out as SEQ ID NO:89 and (amino acids 1-737) and the locations of the associated variable regions and GBS and GH loop regions are defined in Table 5 below. The VP2 capsid protein spans amino acids 138-736 of SEQ ID NO:89 and the VP3 capsid protein spans amino acids 203-736 of SEQ ID NO:89.

(SEQ ID NO: 89)
MAADGYLPDWLEDNLSEGIREWWALKPGAPKPKANQQKQDDGRGLVLPGY
KYLGPFNGLDKGGPVNAADAAALEHDKAYDQQLQAGDNPYLRYNHADAEF
RERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSSLGL
NTMAAGGGAPMADNNEGADGVGSSSGNWHCDSTWLGDRVITTSTRTWALP
TYNNHLYKQISNGTSGGSTNDSTYEGYSTPWGYFDENRSHCHFSPRDWQR
LINNNWGFRPKKLSFKLFNIQVKEVTQNEGTKTIANNLTSTIQVFTDSEY
QLPYVLGSAHQGCLPPFPADVFMIPQYGYLTLNNGGQAVGRSSFYCLEYF
PSQMLRTGNNESFSYIFEDVPFHSSYAHSQSLDRLMNPLIDQYLYYLSRT
QSTGGTAGTQQLLFSQAGPNNMSAQAKNWLPGPMYRQQRVSTTLSQNNNS
NFAWTGGTKYHLNGRDSLVNPGVAMATNKDDEDRFFPSSGVLMFGKQGAG
KDNVDYSSVMLTSEEEIKTTNPVATEQYGVVADNLQQQNTAPIVGAVNSQ
GALPGMVWQNRDVYLQGPIWAKIPHTDGNFHPSPLMGGFGLKHPPPQILI
KNTPVPADPPTAFNQAKLNSFITQYSTGQVSVEIEWELLKESSKRWNPEI
QYTSNYYKSTNVDFAVNTEGVYSEPRPIGTRYLTRNL

The VP1 sequence of AAV5 capsid is set out as SEQ ID NO:5 (amino acids 1-724) and the locations of the associated variable regions and GBS and GH loop regions are defined in Table 5 below. The VP2 capsid protein spans amino acids 137-724 (TAPTGK . . . TRPL) of SEQ ID NO:5 and the VP3 capsid protein spans amino acids 193-724 (MSAGGG . . . TRPL) of SEQ ID NO:5.

The VP1 sequence of AAV8 capsid is set out as SEQ ID NO:9 (amino acids 1-738) and the locations of the associated variable regions and GBS and GH loop regions are defined in Table 5 below. The VP2 capsid protein spans amino acids 138-738 (TAPGKK . . . TRNL) of SEQ ID NO:9 and the VP3 capsid protein spans amino acids 204-738 (MAAGGG . . . TRNL) of SEQ ID NO:9.

The VP1 sequence of AAVBo capsid is set out as SEQ ID NO:1 (amino acids 1-736) and the locations of the associated variable regions and GBS and GH loop regions are defined in Table 5 below. The VP2 capsid protein spans amino acids 140-736 (TAPAAK . . . TNHL) of SEQ ID NO:1 and the VP3 capsid protein spans amino acids 204-736 (MRAAGG . . . TNHL) of SEQ ID NO:1.

The VP1 sequence of Rh10 capsid is set out as SEQ ID NO:12 (amino acids 1-738) and the locations of the associated variable regions and GBS and GH loop regions are defined in Table 5 below. The VP2 capsid protein spans amino acids 138-738 (TAPGKK . . . TRNL) of SEQ ID NO:12 and the VP3 capsid protein spans amino acids 204-738 (MAAGGG . . . TRNL) of SEQ ID NO:12.

The VP1 sequence of LK03 capsid is set out as SEQ ID NO:173 (amino acids 1-738) and the locations of the associated variable regions and GBS and GH loop regions are defined in Table 5 below. The VP2 capsid protein spans amino acids 138-738 (TAPGKK . . . TRNL) of SEQ ID NO:173 and the VP3 capsid protein spans amino acids 204-738 (MAAGGG . . . TRNL) of SEQ ID NO:173.

In Table 5 below, “VR” refers to the variable region and the numbers refer to the amino acid residues each variable region or the GBS and GH loop regions span in the amino acid sequence.

TABLE 5

| AAV | VRI | VRII | VRIII | VRIV | GBS | VRV | VRVI | VRVII | VRVIII | VRIX | GHloop |
|---|---|---|---|---|---|---|---|---|---|---|---|
| AAV2 | 262- | 325- | 380- | 450- | 464- | 487- | 526- | 544- | 580- | 703- | 446- |
| (SEQ ID | 268 | 330 | 384 | 459 | 475 | 504 | 538 | 557 | 592 | 711 | 600 |
| NO: 3) | | | | | | | | | | | |
| AAV4 | 256- | 316- | 372- | 444- | 459- | 481- | 526- | 542- | 579- | 702- | 440- |
| (SEQ ID | 260 | 321 | 378 | 454 | 470 | 504 | 536 | 556 | 591 | 710 | 599 |
| NO: 4) | | | | | | | | | | | |
| AAV5 | 252- | 316- | 372- | 443- | 452- | 474- | 513- | 531- | 570- | 692- | 439- |
| (SEQ ID | 258 | 321 | 377 | 446 | 462 | 491 | 525 | 547 | 582 | 700 | 590 |
| NO: 5) | | | | | | | | | | | |
| AAV6 | 262- | 326- | 381- | 451- | 465- | 488- | 527- | 545- | 581- | 704- | 447- |
| (SEQ ID | 269 | 331 | 385 | 460 | 476 | 505 | 539 | 558 | 593 | 712 | 601 |
| NO: 6) | | | | | | | | | | | |
| AAV6.2 | 262- | 326- | 381- | 451- | 465- | 488- | 527- | 545- | 581- | 704- | 447- |
| (SEQ ID | 269 | 331 | 385 | 460 | 476 | 505 | 539 | 558 | 593 | 712 | 601 |
| NO: 7) | | | | | | | | | | | |
| AAV7 | 263- | 327- | 382- | 452- | 467- | 490- | 529- | 547- | 582- | 705- | 448- |
| (SEQ ID | 270 | 332 | 386 | 462 | 478 | 507 | 541 | 559 | 594 | 713 | 602 |
| NO: 8) | | | | | | | | | | | |
| AAV8 | 263- | 328- | 383- | 453- | 467- | 490- | 529- | 548- | 583- | 706- | 449- |
| (SEQ ID | 271 | 333 | 387 | 462 | 478 | 507 | 541 | 560 | 595 | 714 | 603 |
| NO: 9) | | | | | | | | | | | |
| AAV9 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 10) | | | | | | | | | | | |
| AAVrh8 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 11) | | | | | | | | | | | |
| AAVrh10 | 263- | 328- | 383- | 453- | 467- | 490- | 529- | 548- | 583- | 706- | 449- |
| (SEQ ID | 271 | 333 | 387 | 462 | 478 | 507 | 541 | 560 | 595 | 714 | 603 |
| NO: 12) | | | | | | | | | | | |
| AAVbo | 258- | 318- | 374- | 446- | 462- | 484- | 528- | 544- | 581- | 704- | 442- |
| (SEQ ID | 262 | 323 | 380 | 456 | 472 | 506 | 538 | 558 | 593 | 712 | 601 |
| NO: 1) | | | | | | | | | | | |
| AAVBba.21 | 265- | 325- | 381- | 452- | 468- | 490- | 534- | 550- | 587- | 710- | 448- |
| (SEQ ID | 269 | 330 | 386 | 462 | 478 | 512 | 544 | 564 | 599 | 718 | 607 |
| NO: 15) | | | | | | | | | | | |
| AAVBba.31 | 265- | 325- | 381- | 452- | 468- | 490- | 534- | 550- | 587- | 710- | 448- |
| (SEQ ID | 269 | 330 | 386 | 462 | 478 | 512 | 544 | 564 | 599 | 718 | 607 |
| NO: 20) | | | | | | | | | | | |
| AAVBba.32 | 265- | 325- | 381- | 452- | 468- | 490- | 534- | 550- | 587- | 710- | 448- |
| (SEQ ID | 269 | 330 | 386 | 462 | 478 | 512 | 544 | 564 | 599 | 718 | 607 |
| NO: 21) | | | | | | | | | | | |
| AAVBba.33 | 265- | 325- | 381- | 452- | 468- | 490- | 534- | 550- | 587- | 710- | 448- |
| (SEQ ID | 269 | 330 | 386 | 462 | 478 | 512 | 544 | 564 | 599 | 718 | 607 |
| NO: 22) | | | | | | | | | | | |
| AAVBpo.1 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 53) | | | | | | | | | | | |
| AAVBpo.2 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 54) | | | | | | | | | | | |
| AAVBpo.3 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 55) | | | | | | | | | | | |
| AAVBpo.4 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 56) | | | | | | | | | | | |
| AAVBpo.6 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 57) | | | | | | | | | | | |
| AAVBpo.8 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 58) | | | | | | | | | | | |
| AAVBpo.13 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 59) | | | | | | | | | | | |
| AAVBpo.18 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 60) | | | | | | | | | | | |
| AAVBpo.20 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 61) | | | | | | | | | | | |
| AAVBpo.23 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 62) | | | | | | | | | | | |
| AAVBpo.24 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 63) | | | | | | | | | | | |
| AAVBpo.27 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 64) | | | | | | | | | | | |
| AAVBpo.28 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 65) | | | | | | | | | | | |
| AAVBpo.29 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 66) | , | | | | | | | | | | |
| AAVBpo.33 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 67) | | | | | | | | | | | |
| AAVBpo.35 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 68) | | | | | | | | | | | |
| AAVBpo.36 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 69) | | | | | | | | | | | |
| AAVBpo.37 | 243- | 307- | 363- | 434- | 443- | 465- | 505- | 523- | 562- | 684- | 430- |
| (SEQ ID | 249 | 312 | 368 | 437 | 453 | 483 | 517 | 539 | 574 | 692 | 582 |
| NO: 70) | | | | | | | | | | | |
| AAVmo | 249- | 310- | 366- | 437- | 443- | 467- | 507- | 525- | 556- | 679- | 433- |
| (SEQ ID | 252 | 315 | 371 | 439 | 455 | 484 | 519 | 533 | 568 | 689 | 576 |
| NO: 2) | | | | | | | | | | | |
| AAVBce.14 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 32) | | | | | | | | | | | |
| AAVBce.15 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 33) | | | | | | | | | | | |
| AAVBce.16 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 34) | | | | | | | | | | | |
| AAVBce.17 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 35) | | | | | | | | | | | |
| AAVBce.18 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 36) | | | | | | | | | | | |
| AAVBce.20 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 37) | | | | | | | | | | | |
| AAVBma.42 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 51) | | | | | | | | | | | |
| AAVBma.43 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 52) | | | | | | | | | | | |
| AAVBce.39 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 40) | | | | | | | | | | | |
| AAVBce.40 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 41) | | | | | | | | | | | |
| AAVBce.41 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 42) | | | | | | | | | | | |
| AAVBce.42 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 43) | | | | | | | | | | | |
| AAVanc110 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 602 |
| NO: 14) | | | | | | | | | | | |
| AAVanc80 | 262- | 326- | 381- | 451- | 465- | 488- | 527- | 545- | 581- | 704- | 447- |
| (SEQ ID | 269 | 331 | 385 | 460 | 476 | 505 | 539 | 558 | 593 | 712 | 601 |
| NO: 13) | | | | | | | | | | | |
| AAVBce.35 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 38) | | | | | | | | | | | |
| AAVBce.36 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 39) | | | | | | | | | | | |
| AAVBce.43 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 44) | | | | | | | | | | | |
| AAVBce.44 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 45) | | | | | | | | | | | |
| AAVBce.45 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 46) | | | | | | | | | | | |
| AAVBce.46 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 47) | | | | | | | | | | | |
| AAVBcy.20 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 48) | | | | | | | | | | | |
| AAVBcy.22 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 49) | | | | | | | | | | | |
| AAVBcy.23 | 258- | 322- | 377- | 447- | 460- | 483- | 522- | 540- | 575- | 698- | 443- |
| (SEQ ID | 265 | 327 | 381 | 455 | 471 | 500 | 534 | 552 | 587 | 706 | 595 |
| NO: 50) | | | | | | | | | | | |
| AAVBba.26 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 16) | | | | | | | | | | | |
| AAVBba.27 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 17) | | | | | | | | | | | |
| AAVBba.29 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 18) | | | | | | | | | | | |
| AAVBba.30 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 19) | | | | | | | | | | | |
| AAVBba.34 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 23) | | | | | | | | | | | |
| AAVBba.35 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 24) | | | | | | | | | | | |
| AAVBba.36 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 25) | | | | | | | | | | | |
| AAVBba.37 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 26) | | | | | | | | | | | |
| AAVBba.38 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 27) | | | | | | | | | | | |
| AAVBba.41 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 28) | | | | | | | | | | | |
| AAVBba.42 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 29) | | | | | | | | | | | |
| AAVBba.43 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 30) | | | | | | | | | | | |
| AAVBba.44 | 265- | 328- | 383- | 453- | 469- | 492- | 531- | 549- | 584- | 707- | 449- |
| (SEQ ID | 271 | 333 | 387 | 464 | 480 | 508 | 543 | 561 | 596 | 715 | 604 |
| NO: 31) | | | | | | | | | | | |
| AAVBrh.26 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 71) | | | | | | | | | | | |
| AAVBrh.27 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 72) | | | | | | | | | | | |
| AAVBrh.28 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 73) | | | | | | | | | | | |
| AAVBrh.29 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 74) | | | | | | | | | | | |
| AAVBrh.30 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 75) | | | | | | | | | | | |
| AAVBrh.31 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 76) | | | | | | | | | | | |
| AAVBrh.32 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 77) | | | | | | | | | | | |
| AAVBrh.33 | 262- | 327- | 382- | 452- | 465- | 488- | 527- | 545- | 581- | 704- | 448- |
| (SEQ ID | 270 | 332 | 386 | 460 | 476 | 505 | 539 | 558 | 593 | 712 | 601 |
| NO: 78) | | | | | | | | | | | |
| AAVBfm.17 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 79) | | | | | | | | | | | |
| AAVBfm.18 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 80) | | | | | | | | | | | |
| AAVBfm.20 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 81) | | | | | | | | | | | |
| AAVBfm.21 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 82) | | | | | | | | | | | |
| AAVBfm.24 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 83) | , | | | | | | | | | | |
| AAVBfm.25 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 84) | | | | | | | | | | | |
| AAVBfm.27 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 85) | | | | | | | | | | | |
| AAVBfm.32 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 86) | | | | | | | | | | | |
| AAVBfm.33 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 87) | | | | | | | | | | | |
| AAVBfm.34 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 88) | | | | | | | | | | | |
| AAVBfm.35 | 262- | 327- | 382- | 452- | 466- | 489- | 528- | 546- | 582- | 705- | 448- |
| (SEQ ID | 270 | 332 | 386 | 461 | 477 | 506 | 540 | 559 | 594 | 713 | 602 |
| NO: 89) | | | | | | | | | | | |
| AAVLK03 | 263- | 328- | 383- | 453- | 467- | 490- | 529- | 547- | 583- | 706- | 449- |
| (SEQ ID | 271 | 333 | 387 | 462 | 478 | 507 | 541 | 560 | 595 | 714 | 603 |
| NO: 173) | | | | | | | | | | | |

### Transgene

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, desirably, the transgene is a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See, for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in a host cell. The disclosure further includes using multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Suitable transgenes may be readily selected by one of skill in the art. The selection of the transgene is not considered to be a limitation of this disclosure.

In some embodiments, the transgene is a heterologous protein, and this heterologous protein is a therapeutic protein. Exemplary therapeutic proteins include, but are not limited to, blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α.), transforming growth factor beta (TGF-.β.), and the like; soluble receptors, such as soluble TNF-α. receptors, soluble VEGF receptors, soluble interleukin receptors (e.g., soluble IL-1 receptors and soluble type II IL-1 receptors), soluble .γ/δ T cell receptors, ligand-binding fragments of a soluble receptor, and the like; enzymes, such as α-glucosidase, imiglucarase, β-glucocerebrosidase, and alglucerase; enzyme activators, such as tissue plasminogen activator; chemokines, such as 1P-10, monokine induced by interferon-gamma (Mig), Groa/IL-8, RANTES, MIP-1α, MIP-1β., MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In various embodiments, the heterologous protein is selected from the group consisting of Factor VIII, Factor IX, ATP7B protein, C1 esterase inhibitor (C1-INH), alpha 1 antitrypsin, and galactose-1-phosphate uridyl transferase (GALT), dystrophin, a mini-dystrophin, microdystrophin, phenylalanine hydroxylase (PAH), alpha-galactosidase A, and glucocerebrosidase.

### Regulatory Control Elements

The AAV vector also includes conventional control elements or sequences which are operably linked to the transgene in a manner which permits its transcription, translation and/or expression in a cell transfected with the plasmid vector or infected with the virus described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

In another embodiment, the native promoter for the transgene is used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a gene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

Optionally, plasmids carrying therapeutically useful transgenes may also include selectable markers or reporter genes may include sequences encoding geneticin, hygromicin or purimycin resistance, among others. Such selectable reporters or marker genes (preferably located outside the viral genome to be rescued by the method of production) can be used to signal the presence of the plasmids in bacterial cells, such as ampicillin resistance. Other components of the plasmid may include an origin of replication. Selection of these and other promoters and vector elements are conventional and many such sequences are available [see, e.g., Sambrook et al, and references cited therein].

### Methods for Producing Recombinant AAVs

The present disclosure provides materials and methods for producing recombinant AAVs in insect or mammalian cells. In some embodiments, the viral construct further comprises a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the viral construct further comprises a posttranscriptional regulatory element downstream of the restriction site and upstream of the 3′ AAV ITR. In some embodiments, the viral construct further comprises a polynucleotide inserted at the restriction site and operably linked with the promoter, where the polynucleotide comprises the coding region of a protein of interest. As a skilled artisan will appreciate, any one of the AAV vector disclosed in the present application can be used in the method as the viral construct to produce the recombinant AAV.

In some embodiments, the helper functions are provided by one or more helper plasmids or helper viruses comprising adenoviral or baculoviral helper genes. Non-limiting examples of the adenoviral or baculoviral helper genes include, but are not limited to, E1A, E1B, E2A, E4 and VA, which can provide helper functions to AAV packaging.

Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US Publication No. 20110201088 (the disclosure of which is incorporated herein by reference), helper vectors pHELP (Applied Viromics). A skilled artisan will appreciate that any helper virus or helper plasmid of AAV that can provide adequate helper function to AAV can be used herein.

In some embodiments, the AAV cap genes are present in a plasmid. The plasmid can further comprise an AAV rep gene. The cap genes and/or rep gene from any AAV serotype (including, but not limited to, AAV1, AAV2, AAV4, AAV3, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, and/or variants thereof.) can be used herein to produce the recombinant AAV. In some embodiments, the AAV cap genes encode a capsid from serotype 1, serotype 2, serotype 4, serotype 5, serotype 6, serotype 7, serotype 8, serotype 9, serotype 10, serotype 11, serotype 12, serotype 13 or a variant thereof.

In some embodiments, the insect or mammalian cell can be transfected with the helper plasmid or helper virus, the viral construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Recombinant AAV can also be produced using any conventional methods known in the art suitable for producing infectious recombinant AAV. In some instances, a recombinant AAV can be produced by using an insect or mammalian cell that stably expresses some of the necessary components for AAV particle production. For example, a plasmid (or multiple plasmids) comprising AAV rep and cap genes, and a selectable marker, such as a neomycin resistance gene, can be integrated into the genome of the cell. The insect or mammalian cell can then be co-infected with a helper virus (e.g., adenovirus or baculovirus providing the helper functions) and the viral vector comprising the 5′ and 3′ AAV ITR (and the nucleotide sequence encoding the heterologous protein, if desired). The advantages of this method are that the cells are selectable and are suitable for large-scale production of the recombinant AAV. As another non-limiting example, adenovirus or baculovirus rather than plasmids can be used to introduce rep and cap genes into packaging cells. As yet another non-limiting example, both the viral vector containing the 5′ and 3′ AAV LTRs and the rep-cap genes can be stably integrated into the DNA of producer cells, and the helper functions can be provided by a wild-type adenovirus to produce the recombinant AAV.

### Cell Types Used in AAV Production

The viral particles comprising the AAV vectors described herein may be produced using any invertebrate cell type which allows for production of AAV or biologic products and which can be maintained in culture. For example, the insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. Preferred insect cells are cells from the insect species which are susceptible to baculovirus infection, including High Five, Sf9, Se301, SelZD2109, SeUCR1, Sf900+, Sf21, BTI-TN-5B1-4, MG-1, Tn368, HzAm1, BM-N, Ha2302, Hz2E5 and Ao38.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori (Bm-NPV) (Kato et al., 2010).

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; EP 155,476; Vlak et al (1988); Miller et al (1988); Carbonell et al (1988); Maeda et al (1985); Lebacq-Verheyden et al (1988); Smith et al (1985); Miyajima et al (1987); and Martin et al (1988). Numerous baculovirus strains and variants and corresponding permissive insect host cells that can be used for protein production are described in Luckow et al (1988), Miller et al (1986); Maeda et al (1985) and McKenna (1989).

In another aspect of the disclosure, the methods are also carried out with any mammalian cell type which allows for replication of AAV or production of biologic products, and which can be maintained in culture. Preferred mammalian cells used can be HEK293, HeLa, CHO, NS0, SP2/0, PER.C6, Vero, RD, BHK, HT 1080, A549, Cos-7, ARPE-19 and MRC-5 cells.

### Production of Heterologous Proteins In Vitro

As a non-limiting example, the recombinant AAV disclosed herein can be used to produce a protein of interest in vitro, for example, in a cell culture. As one non-limiting example, in some embodiments, a method for producing a protein of interest in vitro, where the method includes providing a recombinant AAV comprising a nucleotide sequence encoding the heterologous protein; and contacting the recombinant AAV with a cell in a cell culture, whereby the recombinant AAV expresses the protein of interest in the cell. The size of the nucleotide sequence encoding the protein of interest can vary. For example, the nucleotide sequence can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length.

### Production of Heterologous Proteins In Vivo

The recombinant AAV disclosed herein can be used to produce a protein of interest in vivo, for example in an animal such as a mammal. Some embodiments provide a method for producing a protein of interest in vivo, where the method includes providing a recombinant AAV comprising a nucleotide sequence encoding the protein of interest; and administering the recombinant AAV to the subject, whereby the recombinant AAV expresses the protein of interest in the subject. The subject can be, in some embodiments, a non-human mammal, for example, a monkey, a dog, a cat, a mouse, or a cow. The size of the nucleotide sequence encoding the protein of interest can vary. For example, the nucleotide sequence can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length.

### Therapeutic Uses

The recombinant AAV produced by the methods described herein can be used to express one or more therapeutic proteins to treat various diseases or disorders. Non-limiting examples of the diseases include cancer such as carcinoma, sarcoma, leukemia, or lymphoma. Additional diseases that can be treated using the AAV vectors, recombinant viruses and methods disclosed herein include genetic disorders including sickle cell anemia, cystic fibrosis, lysosomal acid lipase (LAL) deficiency 1, Tay-Sachs disease, Phenylketonuria, Mucopolysaccharidoses, Glycogen storage diseases (GSD, e.g., GSD types I, II, III, IV, V, VI, VII, VIII, IX, X, XI, XII, XIII, and XIV), Galactosemia, muscular dystrophies (e.g., Duchenne muscular dystrophy), hemophilia such as hemophilia A (classic hemophilia) and hemophilia B (Christmas Disease), Wilson's disease, hereditary angioedema (HAE), alpha 1 antitrypsin deficiency, phenylketonuria (PKU), Fabry Disease, and Gaucher Disease.

In various embodiments, the disorder or disease is selected from the group consisting of hemophilia A, hemophilia B, Wilson's disease, hereditary angioedema (HAE), alpha 1 antitrypsin deficiency, galactosemia, Duchenne's Muscular Dystrophy or other muscular dystrophies, phenylketonuria (PKU), Fabry Disease, and Gaucher Disease. Additional disorders or diseases contemplated herein include those that can be treated either by local expression in the liver or in muscle, or by expression of secreted protein from the liver (or muscle). The amount of the heterologous protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the protein of interest is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein of interest is needed for the method to be effective can vary depending on non-limiting factors such as the particular protein of interest and the subject receiving the treatment, and an effective amount of the protein can be readily determined by a skilled artisan using conventional methods known in the art without undue experimentation.

Contemplated herein is a method of treating a subject having a disease or disorder as described herein with multiple doses of a recombinant adeno-associated virus (rAAV) vector, the method comprising: administering to a subject a first rAAV vector comprising a transgene and a first capsid protein, and administering to a subject a second rAAV vector with a second capsid protein comprising the same transgene as the first gene therapy vector.

In addition, comtemplated herein is a first rAAV vector for use in a method of treating a subject with multiple doses of rAAV vector, wherein the method comprises: (a) administering to the subject the first rAAV vector, wherein the first rAAV vector comprises a transgene comprising a therapeutic molecule and a first capsid, and (b) administering to the subject a second rAAV vector, wherein the second rAAV vector comprises a transgene and a second capsid, wherein the transgene in the second rAAV vector comprises the same therapeutic molecule or a different therapeutic molecule as the transgene in the first rAAV vector.

Also contemplated herein is a first rAAV vector for use in a method of treating a disease or disorder in a subject in need thereof with multiple doses of rAAV vector, the method comprising: (a) administering to the subject the first rAAV vector comprising a transgene comprising a therapeutic molecule useful for treating the disease or disorder and a first capsid, and (b) administering to a subject a second rAAV vector comprising a transgene comprising a therapeutic molecule useful for treating the disease or disorder and a second capsid, wherein the transgene in the second rAAV vector comprises the same therapeutic molecule or a different therapeutic molecule useful to treat the disease or disorder as the transgene in the first rAAV vector.

In addition, comtemplated herein is a first rAAV vector for use in a gene therapy method which involves the administration of multiple doses of rAAV vector, wherein the method comprises: (a) administering to a subject the first rAAV vector, wherein the first rAAV vector comprises a transgene comprising a therapeutic molecule and a first capsid, and (b) administering to the subject a second rAAV vector, wherein the second rAAV vector comprises a transgene and a second capsid, wherein the transgene in the second rAAV vector comprises the same therapeutic molecule or a different therapeutic molecule.

Also contemplated herein is a a first rAAV vector for use in a gene therapy method which involves the administration of multiple doses of rAAV vector, the method comprising: (a) administering to a subject in need thereof the first rAAV vector comprising a transgene comprising a therapeutic molecule useful for treating the disease or disorder and a first capsid, and (b) administering to the subject a second rAAV vector comprising a transgene comprising a therapeutic molecule useful for treating the disease or disorder and a second capsid, wherein the transgene in the second rAAV vector comprises the same therapeutic molecule or a different therapeutic molecule useful to treat the disease or disorder as the transgene in the first rAAV vector.

It is further contemplated that the first and second capsid proteins are phylogenetically distinct capsid proteins. It is contemplated that the second or subsequent AAV comprises a capsid having sufficient phylogenetic distance between the viruses that there is not significant cross-reactivity of preexisting immunogenicity in the subject against the second capsid protein. In various embodiments, the first capsid protein is selected from the group consisting of AAV1, AAV2, AAV4, AAV3, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, and/or variants thereof. In various embodiments, the second capsid protein is selected from the group consisting of AAV1, AAV2, AAV4, AAV3, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9 VR, AAV_go.1, AAVs listed in Table 4, AAV listed in Table 5, and/or variants thereof.

In some embodiments, the first rAAV vector, the second rAAV vector or both are administered to a subject at a dose of from about 1×10⁹ vg/kg to about 1×10¹⁵ vg/kg of body weight. In certain embodiments, the first rAAV vector, the second rAAV vector or both are administered to a subject at a dose of from about 1×10¹² vg/kg to about 1×10¹⁵ vg/kg of body weight.

In various embodiments, the subject is administered an immunosuppressant, prior to or subsequent to administration of the second gene therapy vector. In various embodiments, the immunosuppressant is selected from the group consisting of T cell inhibitors, calcineurin inhibitors, mTOR inhibitor and steroids. In various embodiments, the immunosuppressant is anti-thymocyte globulin (ATG), tacrolimus, cyclosporine, mycophenolate mofetil, mycophenolate sodium, azathioprine, sirolimus (rapamycin), or prednisone. In various embodiments, the immunosuppressant is delivered via a delivery vehicle, such as a liposome or nanoparticle.

In various embodiments, the subject is administered intravenous immunoglobulins (IVIG) prior to or subsequent to administration of the second gene therapy vector.

In various embodiments, the second gene therapy vector is administered 6 months, 1 year, 1.5 year, 2 years, 2.5 years, 3 years, 4 years, 5 years or 6 years or more after the first administration of a gene therapy vector.

In various embodiments, the gene therapy is administered intravenously or subcutaneously.

### Kits

Contemplated herein is a pharmaceutical kit comprising a first rAAV vector, in a first container; and a second rAAV vector, in a second container, wherein the first rAAV vector comprises a transgene comprising a therapeutic molecule and a first capsid and the second rAAV vector comprises a transgene comprising a therapeutic molecule and a second capsid, wherein the transgene in the second rAAV vector comprises the same or different therapeutic molecule as the transgene in the first rAAV vector. In a specific embodiments, the first capsid is phylogenetically distinct from the second capsid of the second rAAV vector. In certain embodiments, the concentration of the first rAAV in the first container is from about 1×10¹² vg/mL to about 1×10¹⁵ vg/mL. In certain embodiments, the concentration of the second rAAV located in the second container is from about 1×10¹² vg/mL to about 1×10¹⁵ vg/mL. In certain embodiments, the kit comprises instructions for using the kit in a method for the treatment of a disease or disorder in a subject in need thereof, or in a gene therapy method involving multiple administrations of AAV. In certain embodiments, the instructions comprise administration methods. In certain embodiments, the instructions comprise dosing methods. In certain embodiments, the dosing methods comprise timing for dosing a subject with the first rAAV vector, the second rAAV vector, or both.

The following Examples are meant to be illustrative rather than limiting.

### Example 1: Analysis of AAV Capsid Pre-Existing Immunity

One side-effect of gene therapy administration can be the immune response generated against the viral capsid proteins in the gene therapy vector. In addition, humans may have been exposed to AAV and exhibit pre-existing immunity to some capsids that may limit the transduction by the vector. However, some gene therapy vectors may exhibit lower pre-existing immunity compared to other gene therapy vectors. For example, Liu et al. (Gene Ther. 21(8):732-8, 2014) previously described that pre-existing immunity to AAV5 was lower in healthy Chinese populations compared to AAV2 or AAV8. A low-preexisting immunity to the viral vector may be helpful and preferable when determining whether a gene therapy vector can be administered or redosed to a subject without eliciting an immune cascade that limits the efficacy of the vector and reduces the amount of transgene expressed to the subject.

In order to determine which AAV vectors may have the lowest pre-existing immunity, neutralization assays were conducted to determine the amount of transduction inhibition activity to various capsids in average human serum. IVIG, which is IgG pooled from 5000 individuals, is used to evaluate the average pre-existing immunity in human serum. Briefly, 293T cells were uniformly seeded in 96 well, opaque white plates at 4×10⁴ cells/well 20 hours prior to vector addition. Dilutions of 100 mg/mL IVIG (Gammagard) prepared by 2-fold serial dilution into DMEM+1% BSA, from 20 mg/mL to 0.04 mg/mL and a 0 mg/mL control. Vector solutions prepared by dilution of vector stock to 4×10⁹ vg/mL in DMEM+1% BSA+100 uM Etoposide. All vectors were packaged with the same RSV-Firefly Luciferase reporter, purified by double-CsCl gradient and vg/mL quantified by qPCR titer. Consistent total capsid protein was assessed by silver stain gel. IVIG dilutions and vector solutions were mixed 1:1 and incubated at 37° C. for 1 hour. 20 ul/well from the mix plate was subsequently added to the corresponding well in a previously prepared cell plate, in duplicate. Cell plates were incubated at 37° C. for 72 hours. Firefly luciferase expression was quantified by RLU after substrate addition and cell lysis and viewed as relative expression to the 0 mg/mL IVIG control. Neutralizing titers (NC50) were calculated by the concentration of IVIG that results in 50% gene transduction of the cells. Neutralizing titers from serum and plasma from mice, NHPs and humans were generated using minor variations of this general protocol.

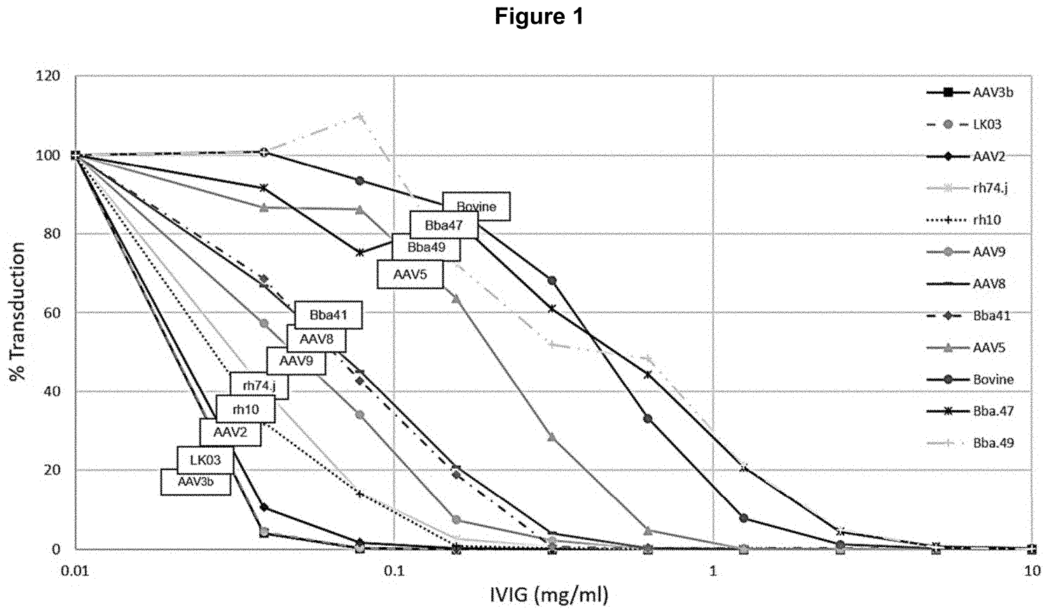

FIG. 1 illustrates the pre-existing immunity to various gene therapy vectors comprising AAV capsids described herein and known in the literature. FIG. 1 shows that human IVIG contains less neutralizing antibodies to bovine, Bba.49, AAV5 and Bba.47 than to the other AAVs.

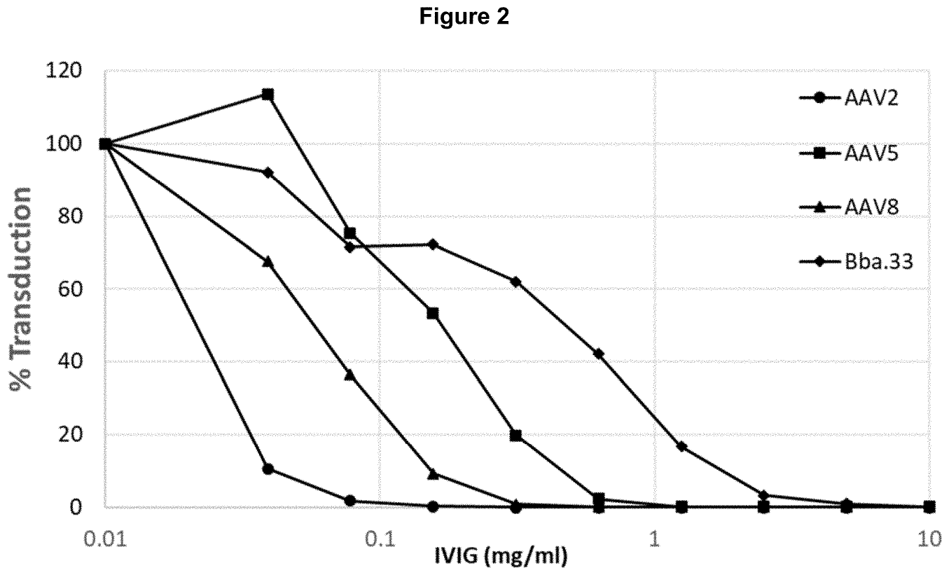

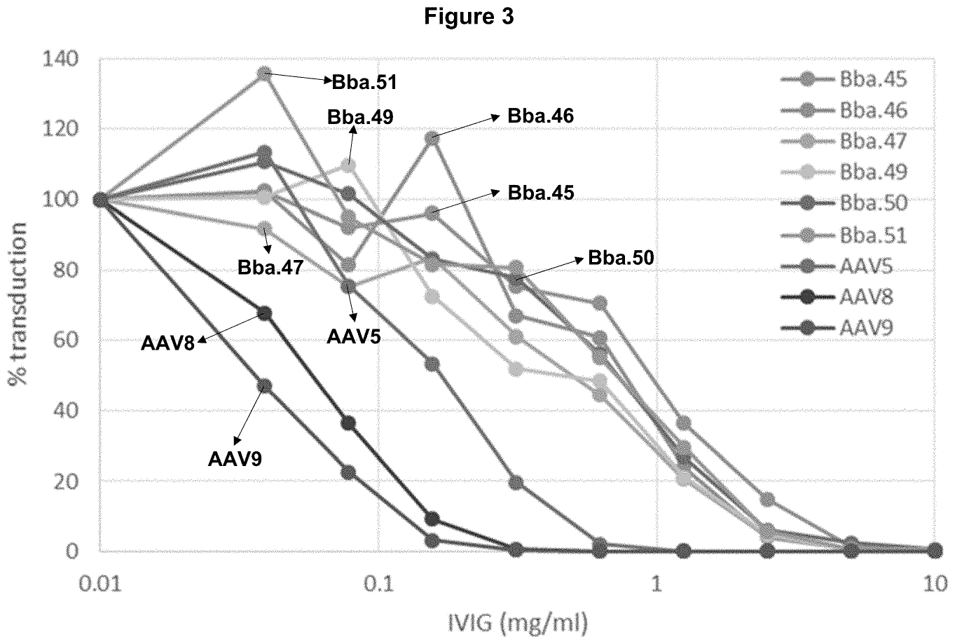

FIG. 2 shows that humans have lower pre-existing immunity to Bba.33 vector compared to AAV2, AAV8 and AAV5. Similarly, FIG. 3 shows that humans also have lower pre-existing immunity to AAV12-like variants Bba.45, Bba.45, Bba.46, Bba.47, Bba.49, Bba.50, and Bba.51 compared to AAV5, AAV8 and AAV9.

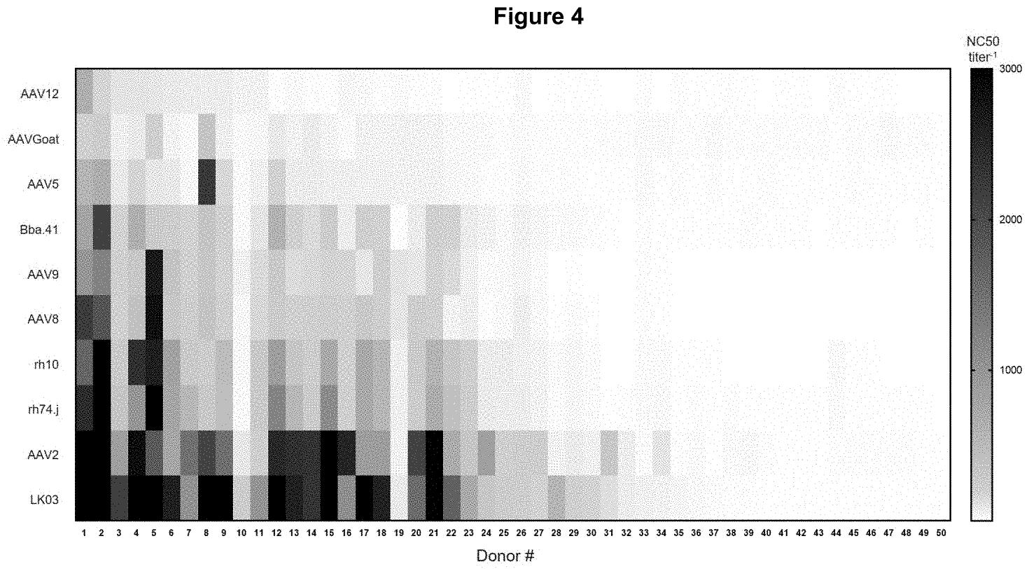

FIG. 4 shows titers of pre-existing neutralizing antibodies to different AAV vectors in individual human donors. The heat map is shaded by titer as described in the legend at the right. The higher the titer, the darker the shading. FIG. 4 demonstrates that humans have pre-existing NAb titers to multiple capsids, and the specificity of those titers closely follows AAV VP3 phylogeny. For example, if a human subject has a pre-existing NAb titer to AAV5 or AAV12, the data suggest they will also have a NAb titer to the common NHP/Human AAV isolates. AAV12-specific antibody titers were generally similar to or lower than AAV5 in all of the 50 human samples tested.

### Example 2: Cross-Reactivity of Capsid Proteins In Vivo

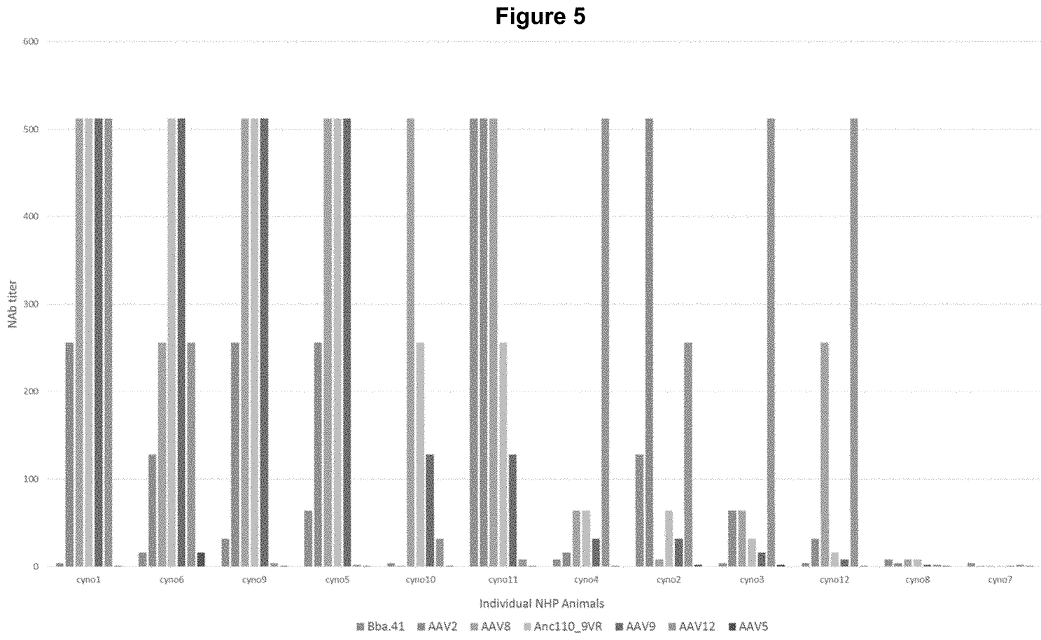

In order to determine if redosing with a second gene therapy vector comprising a different AAV capsid would be possible, cross reactivity to a second vector was evaluated in subjects who have received a first AAV vector expressing a transgene. Pre-existing NAb screening in Non-Human Primates (NHPs) have shown low AAV5 seropositivity but high titers for other “primate capsids”. FIG. 5 shows the results of assays for pre-existing NAb titers for AAV9, AAV12 and other capsids. All animals were dosed with 6×10¹³ vg/kg AAV5-FVIII and examined over 8 weeks for plasma expression of hFVIII protein.

In a further experiment, NHPs with or without pre-existing immunity to AAV5, that received AAV5-FVIII, were assayed for cross-reacting neutralizing antibodies to Bba.49, pre and post AAV5-FVIII dose. NHPs were evaluated for pre-existing antibodies to AAV5 using a total antibody assay (TAb) and inhibitors of transduction were determined using a cell-based in vitro TI assay as previously described. (see e.g. Falese L et al. (2017) Gene Therapy; Sandberg H et al. (2001) Thromb Haemost; 85(1): 93-100). Total binding antibody (TAb) against AAV5 were detected in plasma using a sandwich electrochemiluminescence assay (ECLA) on the MSDTM platform. The cell based transduction inhibition assay tests ability of plasma to block the in vitro transduction of HEK293T/17 cells by a AAV5-CMV-GFP vector.

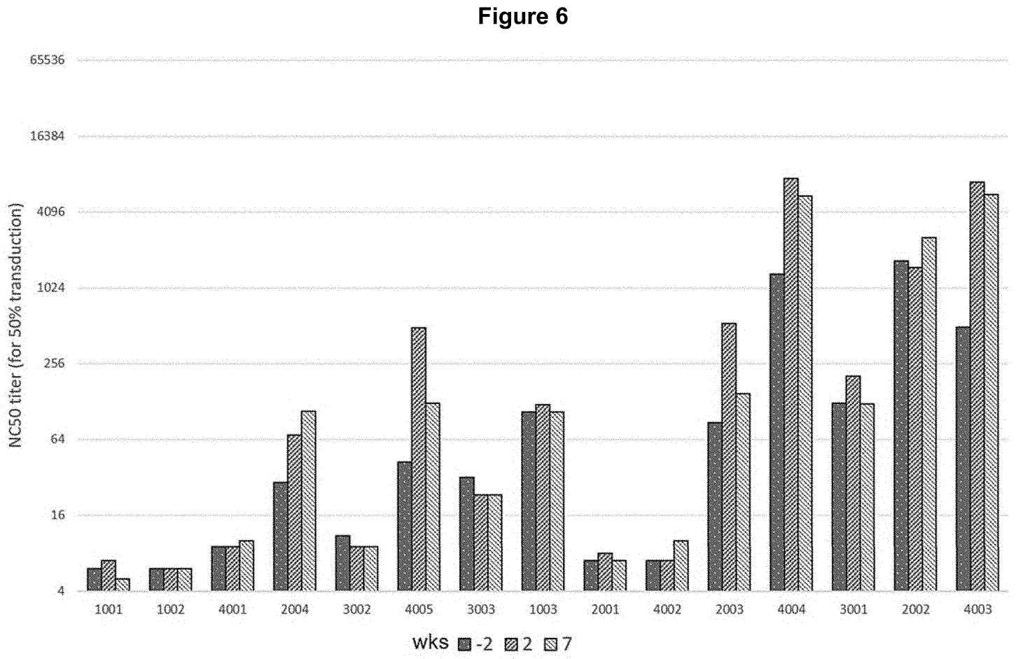

FIG. 6 shows neutralizing antibody titers to Bba.49 in all dosed NHPs, pre AAV5 dose (−2 weeks) and at 2 weeks post AAV5 dose and 7 weeks post AAV5 dose. Pre-existing neutralizing antibody titer to Bba.49 did not significantly affect the cross-reactive titer to Bba.49 post the AAV5 dose.

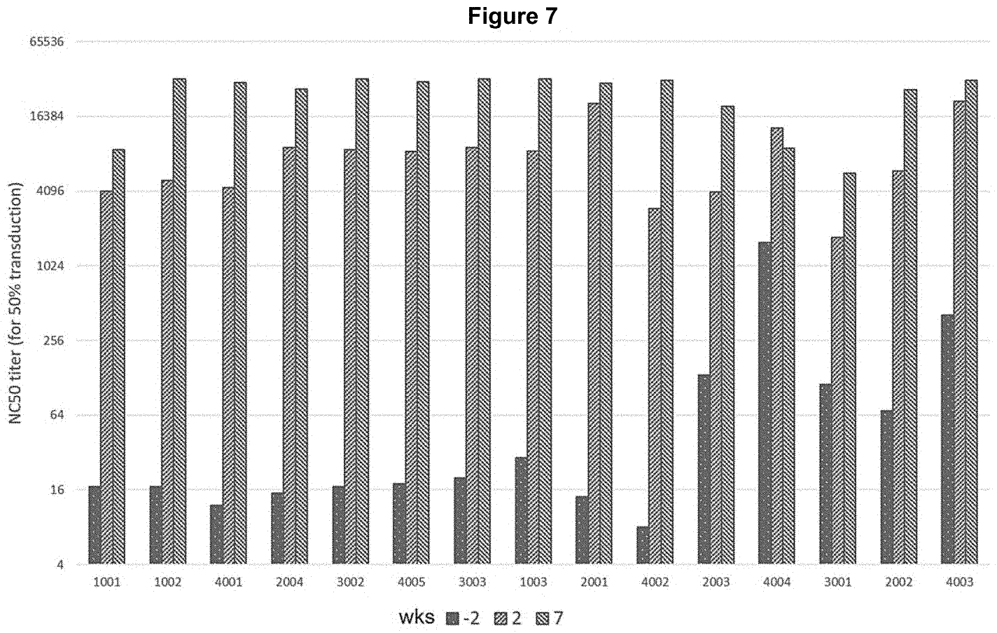

FIG. 7 shows levels of antibody titers to AAV5 at −2 weeks (pre-dose and at 2 weeks post AAV5 dose and 7 weeks post AAV5 dose in NHPs with varying levels of pre-existing neutralizing antibodies to AAV5. As expected, all animals dosed with AAV5 vector produced a robust and sustained anti-AAV5 humoral response, whether or not they had a pre-existing anti-AAV5 titer.

In 9/15 animals, the administration of AAV5 vector did not significantly alter the neutralizing titer to Bba49. 6/15 animals had a pre-existing titer of <20 and none of those showed a significant increase in titer. 9/15 animals had a pre-existing titer >20 and 5 of those animals showed only a modest increase in titer at either 2 or 7 wks post-AAV5 dose. These increases (10 fold) are small relative to the specific anti-AAV5 titers generated from the AAV5 vector, seen in FIG. 7. It is possible the few measured increases in Bba49 neutralizing titers could be due to an indirect boosting of pre-existing Ig levels and/or an antibody affinity maturation process that is inherently broad, both are likely transient and unlikely relevant for clinical redosing applications. That does not preclude the possibility of shared neutralizing epitopes between AAV5 and Bba49, but most of the neutralizing activity generated from the AAV5 vector was conclusively not shared. The lack of any Bba49 cross-reactive neutralizing titers generated post-AAV5 dosing in animals with low pre-existing anti-Bba49 titers and the modest increase in a few animals with elevated pre-existing anti-Bba49 titers supports the conclusion of limited humoral cross-reactivity between AAV5 and Bba49.

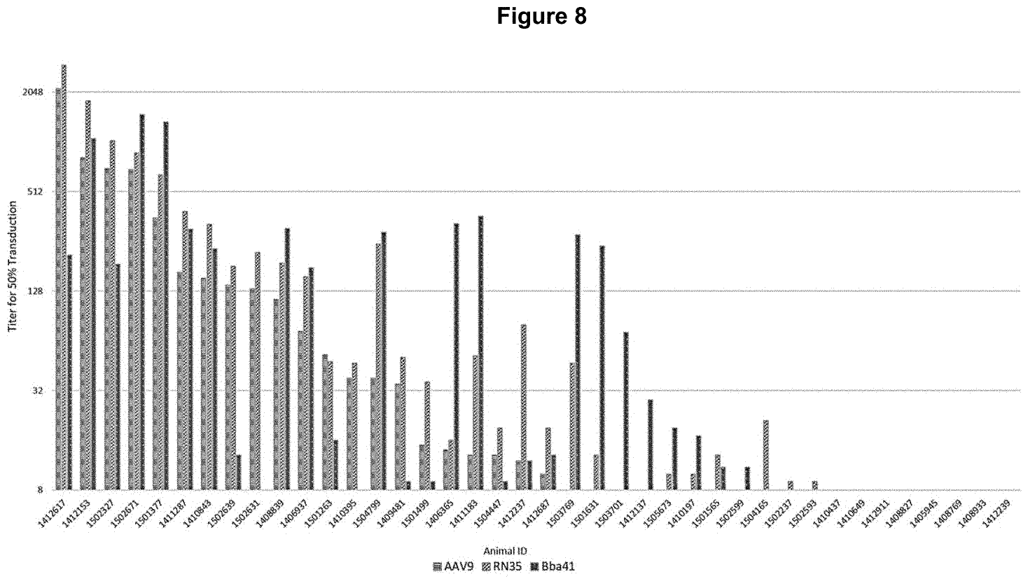

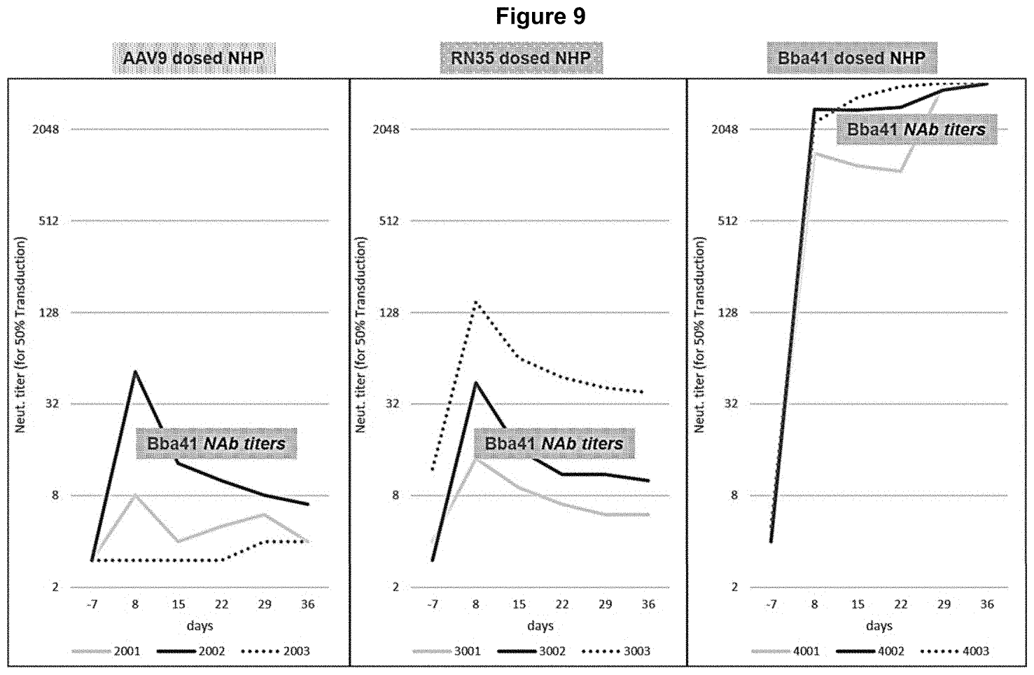

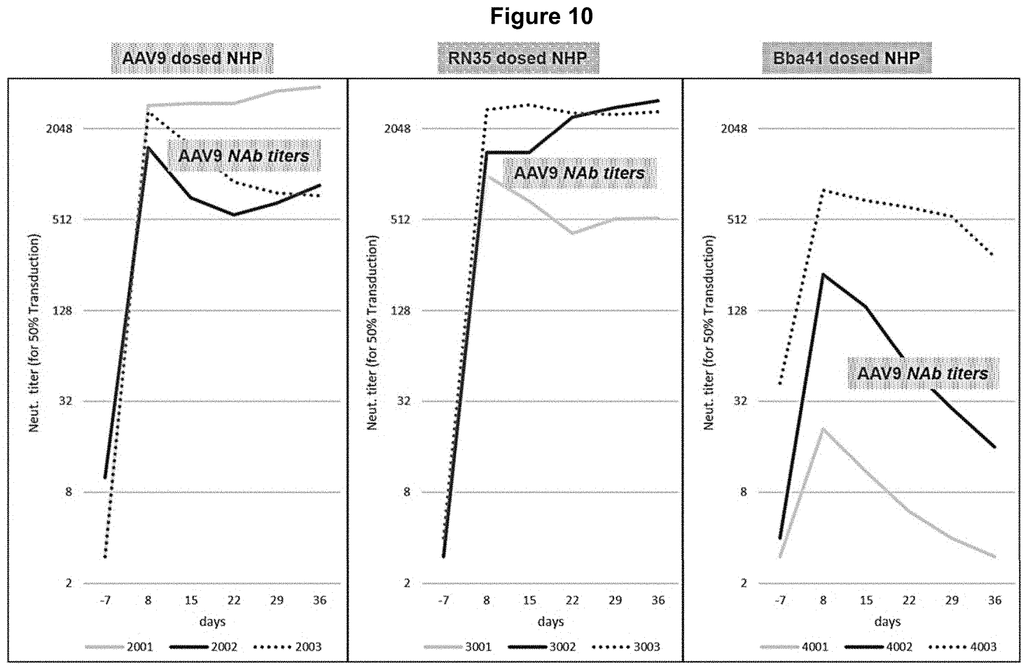

Pre-existing neutralizing antibodies to AAV9, RN35 and Bba.41 were evaluated in NHPs. NHPs have high sero-prevalence for both AAV9 and Bba.41, although some NHP samples showed pre-existing titers specific for one or the other capsid (FIG. 8), suggesting that AAV9 and Bba.41 may be serologically distinct. Neutralizing activity to Bba.41 was tested in NHPs previously dosed with AAV9, RN35 or Bba.41. In NHPs with no pre-existing titers, neutralizing activity shows specificity to the AAV dosed. Only NHPs dosed with Bba.41 showed neutralizing antibodies to Bba.41 (FIG. 9). Cross-reactivity is demonstrated for highly similar capsid proteins (FIG. 10). For example, AAV9 and RN35 have highly similar capsid protein sequences (92.9% sequence ID; RN35 was designed to mimic the capsid surface of AAV9). NHPs dosed with AAV9 show cross-reactivity to RN35 as expected. NHPs dosed with AAV9, which has less sequence identity to Bba.41, show limited cross-reactivity to Bba.41. These results suggest that captive NHPs seem to have more specific pre-existing NAb titers, hypothetically due to more isolated/acute exposures to AAV.

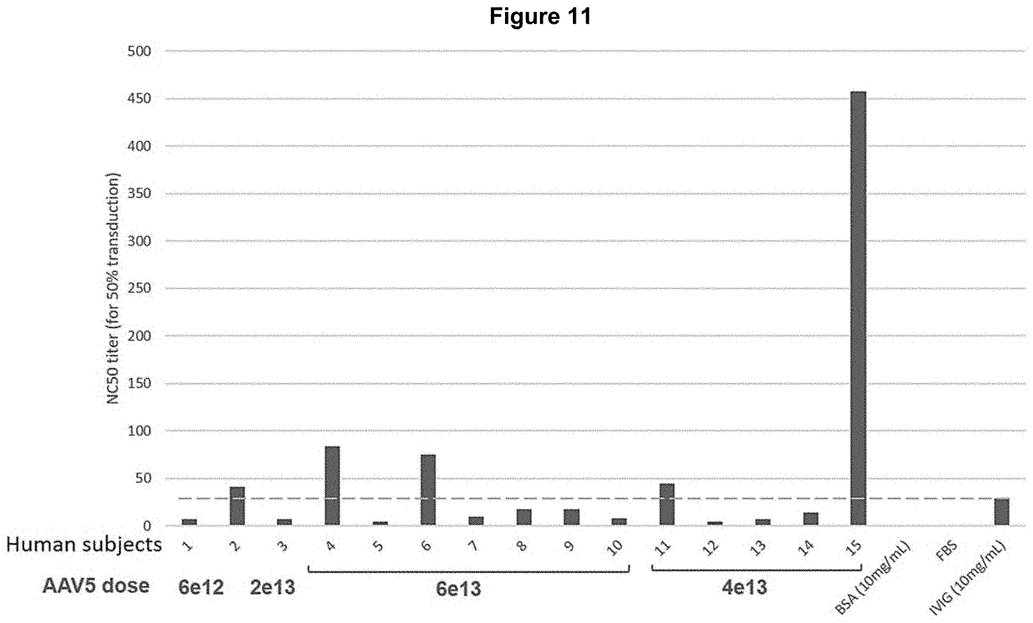

Neutralizing titers to Bba.49 were measured in human patients receiving AAV5-FVIII therapy at 6×10¹² vg/kg, 2×10¹³ vg/kg, 4×10¹³ vg/kg or 6×10¹³ vg/kg (FIG. 11). Serum samples from human subjects receiving AAV5-FVIII therapy during a clinical trial obtained 1.5 to 2.5 years after initial dosing were assayed for inhibition of transduction by Bba.49 in vitro. The neutralizing titer in IVIG represents the average titer in human serum as negative control for comparison. Overall low NAb titers to Bba.49 post-AAV5 dose indicate limited cross-neutralizing activity. One patient had an elevated titer but it is unknown if the titer was pre-existing or caused by AAV5 dose.

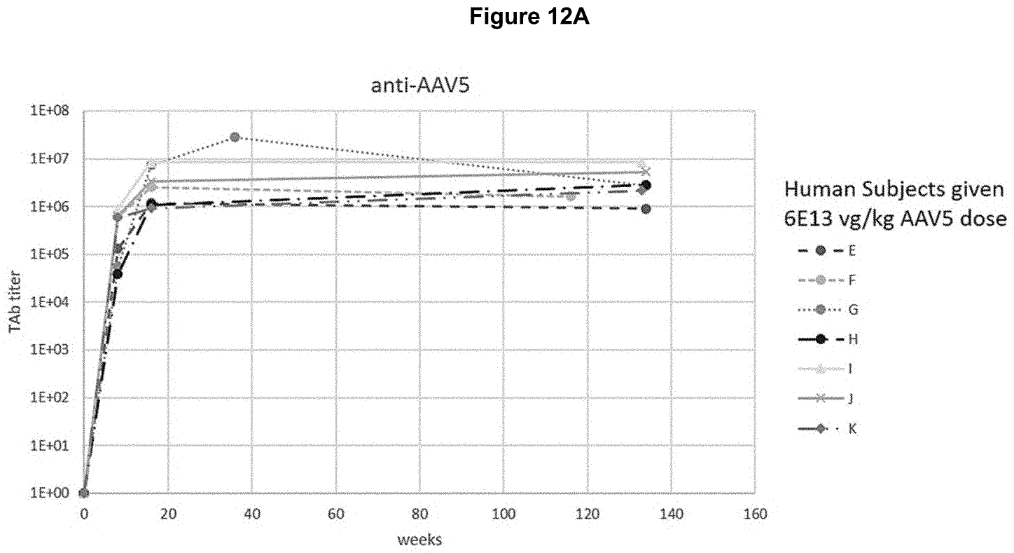

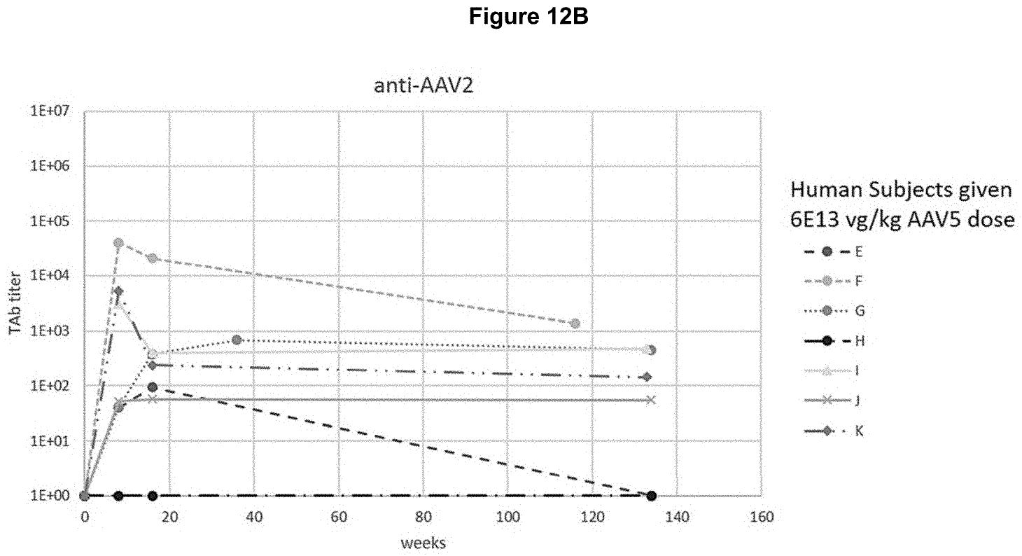

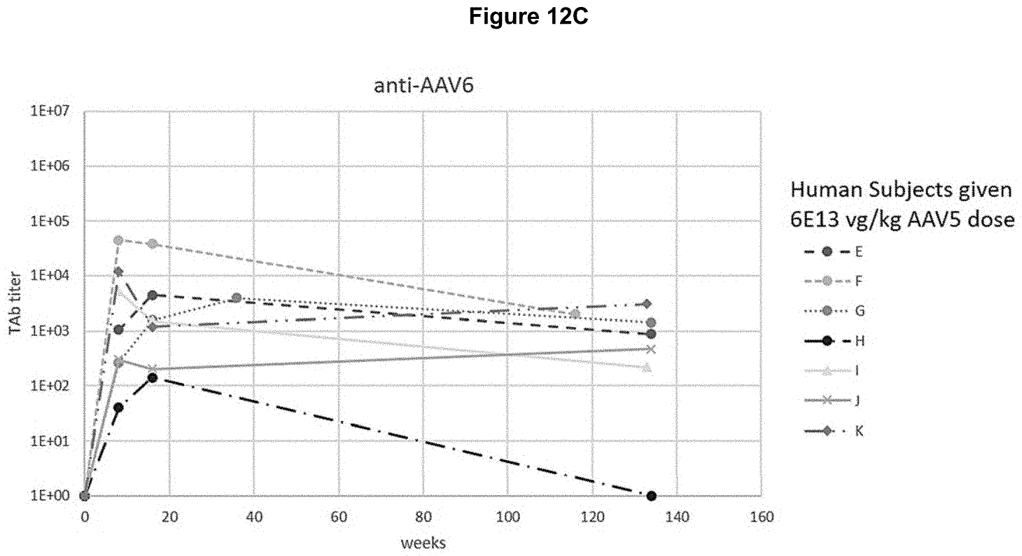

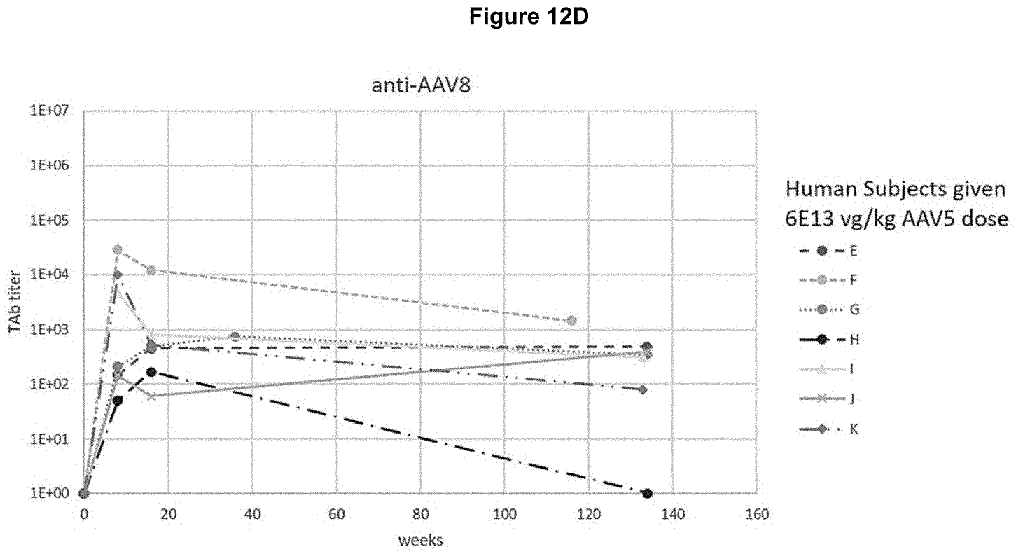

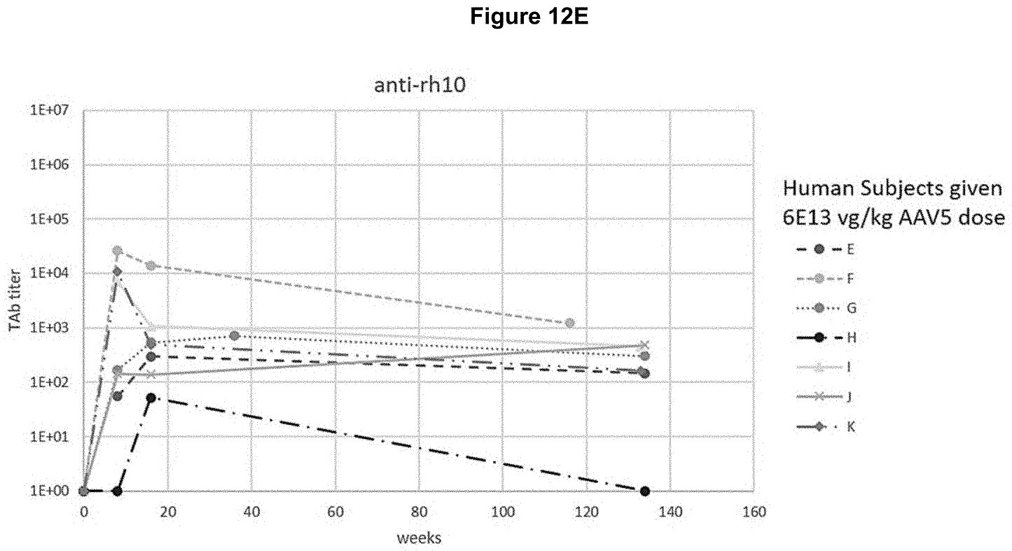

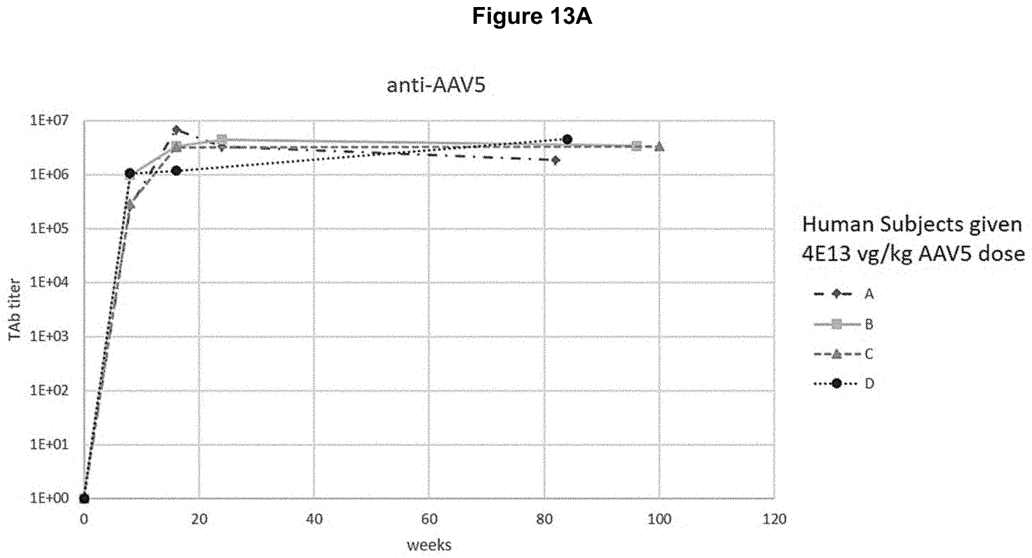

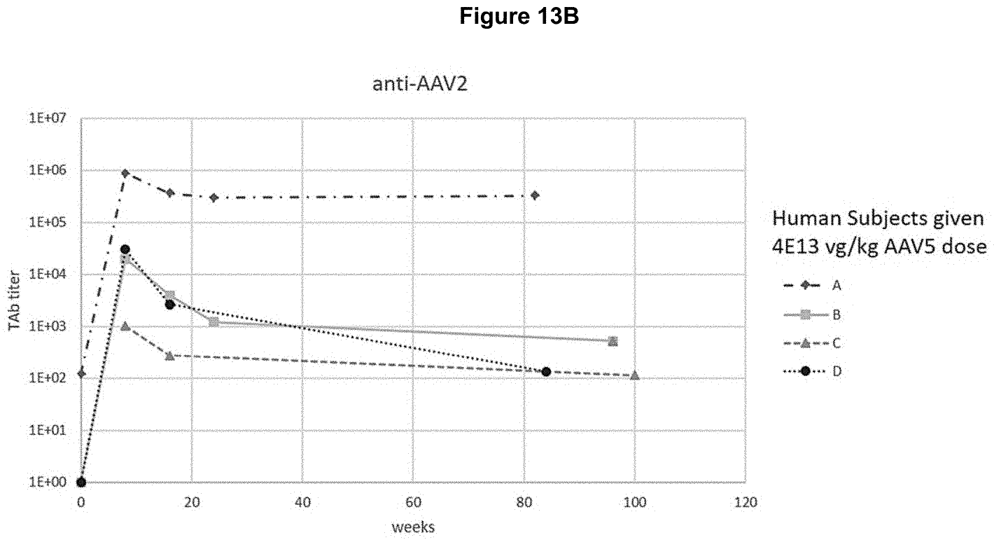

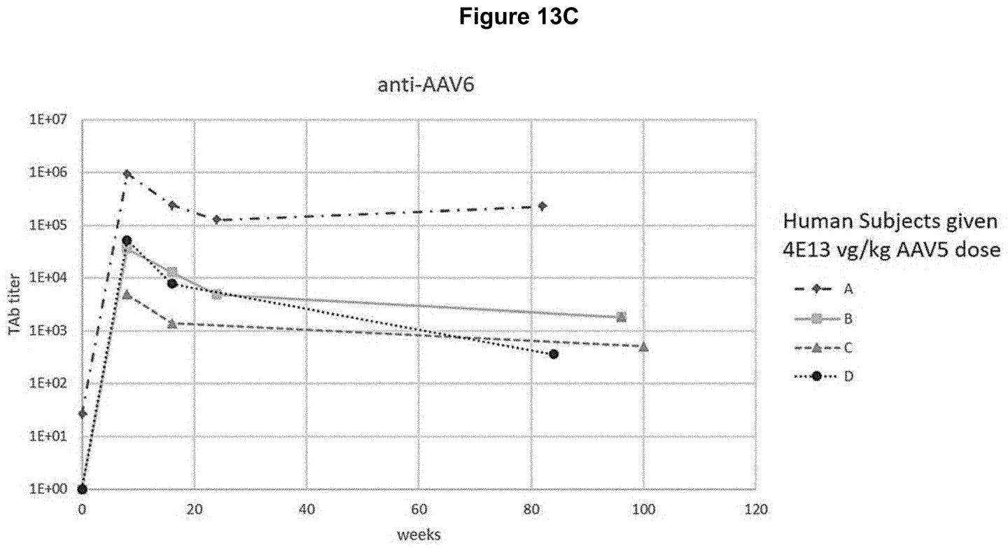

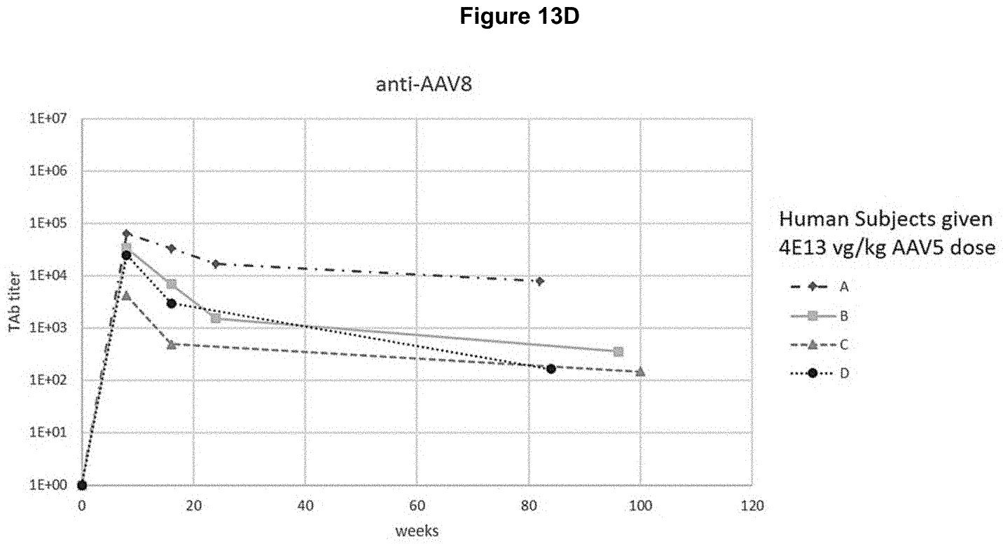

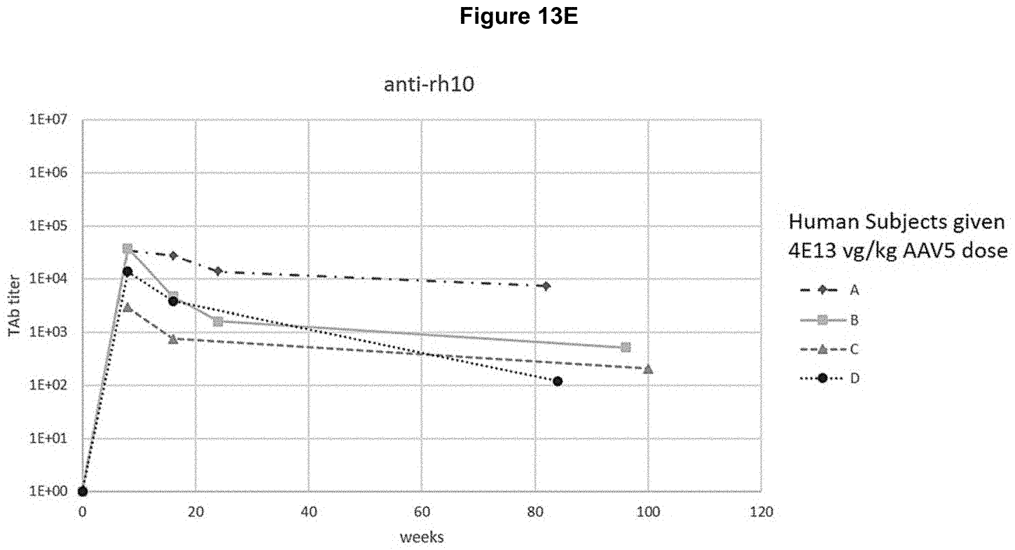

Additional cross-reactivity to different capsids (AAV5, AAV2, AAV6, AAV8 and AAVrh10) was measured in human patients receiving AAV5-FVIII therapy at either 4×10¹³ vg/kg (FIGS. 13A-E) or 6×10¹³ vg/kg (FIGS. 12A-E), pre-dose and at 8 weeks and 1.5-2 yrs post dose. Following dosing, AAV5 antibody titers rise into the tens/hundreds of thousands by week 8 and elevate to millions by 1.5-2.5 years post dose in humans. Antibodies against other capsids are generally at “pre-existing” titer levels 1.5-2.5 yrs post-dose (FIGS. 12A-E-13A-E). However, one patient in the 4×10¹³ vg/kg group had pre-existing titers to both AAV2 and AAV6 and exhibited anti-AAV2 and AAV6 titers well above their pre-existing TAb+ levels (FIGS. 13B-C).

### Example 3-Dual Dosing/Redosing Experiments In Vivo

In order to determine the feasibility of redosing of animals with a different AAV gene therapy vector, experiments in mice were undertaken.

Wild type male mice (C57BL/6J, Jackson Laboratories #000664), 8-10 weeks old, were injected intravenously with 6×10¹³ vg/kg (4 ul/gm) of a luciferase (LUC) gene, contained in either AAV5 or AAV9 serotype capsids. Four weeks following this administration, the mice treated with AAV5-luciferase were injected intravenously with 6×10¹³ vg/kg of β-chain of chorionic gonadotropin (βCG) gene contained in either an AAV5, Bba.47 or Bba.49 capsid (4 ul/gm). Mice treated initially with AAV9-luciferase were injected with 6×10¹³ vg/kg (4 ul/gm) β-CG gene in either an AAV9 or Bba.41 capsid.

Lysates and serum samples were suspended in buffer containing 8M urea and a stable isotope peptide of L*LEPADNPFLPQ (SEQ ID NO: 172) (Pepscan) Samples where then reduced with dithiothreitol (DTT) (Sigma), alkylated with iodoacetamide (IAA) (VWR) and digested with Trypsin/Lys-C (Promega) overnight at 37° C. Digestion was quenched with 10% formic acid. Digested samples were cleaned up using Waters Sep-Pak C18 SPE plate on a Waters positive pressure manifold. Eluted samples were dried using a Rotovap (Thermo) and resuspended in 0.1% formic acid in water prior to injection on a Waters H-Class UPLC connected to a 6500 AB Sciex. Peak integration was performed using Sciex MultiQuant software.

Blood samples were collected just prior to study start and every 2 weeks during the experiment by making a transverse nick across one of the lateral veins of the tail, approximately 0.1 cm in length, using a sterile surgical blade. This yielded a blood sample of approximately 100 uL which was collected and centrifuged (5000 rpm for 10 minutes at 4° C.) to yield serum. Serum was decanted into 1.5 mL eppendorf tubes, stored at −80° C., and transferred for analysis of βCG expression and in vitro transduction inhibition. Animals were killed 12 weeks after the initial AAV5-luciferase or AAV9-luciferase administration, and liver, heart, and gastrocnemius were collected and stored for possible future analysis.

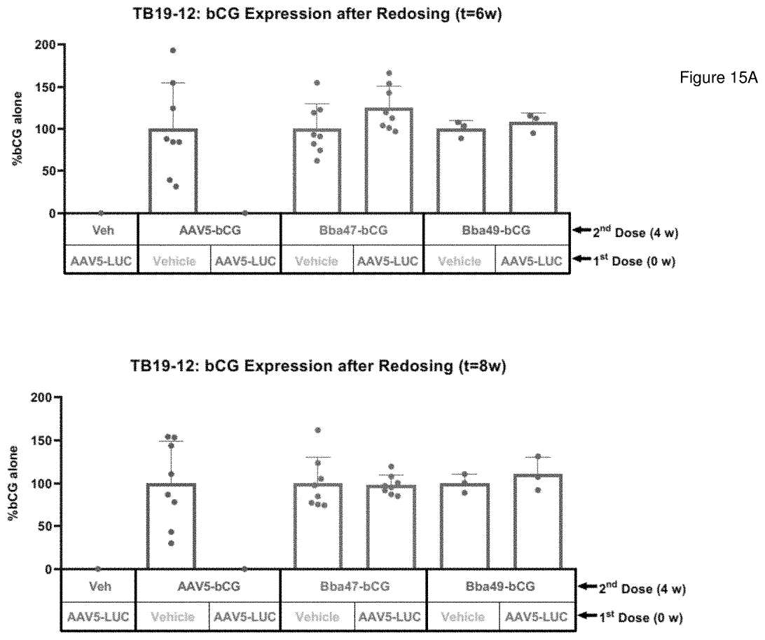

FIG. 15A shows that AAV5-βCG dosed 4 weeks after AAV5-LUC does not produce any measurable βCG in serum at 6 weeks or 8 weeks post administration. In contrast, Bba.47-βCG and Bba.49-βCG, dosed 4 weeks after AAV5-LUC produced equivalent βCG serum expression to mice that were predosed with vehicle alone. Similarly, FIG. 15B shows AAV9-βCG dosed after vehicle produced βCG expression in serum, while AAV9-βCG, dosed after AAV9-LUC did not. In contrast, Bba.41βCG produced βCG expression when predosed with vehicle or AAV9-LUC.

In summary, these studies would suggest that Bba.47 and Bba.49 can be dosed in animals after AAV5 treatment without any considerable neutralizing effects. Similarly, Bba.41-βCG can be dosed after AAV9-LUC without any considerable loss in βCG expression.

TABLE 6
(VP1 sequences (amino acid sequences and representative nucleic acid-
encoding sequences)

| SEQ ID | | |
|---|---|---|
| NO. | AAV | Sequence |
| 1 | AAVbo | MSFVDHPPDWLESIGDGFREFLGLEAGPPKPKANQQKQDNARGLVLPGYK |
| | | YLGPGNGLDKGDPVNFADEVAREHDLSYQKQLEAGDNPYLKYNHADAEFQ |
| | | EKLASDTSFGGNLGKAVFQAKKRILEPLGLVETPDKTAPAAKKRPLEQSP |
| | | QEPDSSSGVGKKGKQPARKRLNFDDEPGAGDGPPPEGPSSGAMSTETEMR |
| | | AAAGGNGGDAGQGAEGVGNASGDWHCDSTWSESHVTTTSTRTWVLPTYNN |
| | | HLYLRLGSSNASDTFNGFSTPWGYFDFNRFHCHFSPRDWQRLINNHWGLR |
| | | PKSMQVRIFNIQVKEVTTSNGETTVSNNLTSTVQIFADSTYELPYVMDAG |
| | | QEGSLPPFPNDVFMVPQYGYCGLVTGGSSQNQTDRNAFYCLEYFPSQMLR |
| | | TGNNFEMVYKFENVPFHSMYAHSQSLDRLMNPLLDQYLWELQSTTSGGTL |
| | | NQGNSATNFAKLTKTNFSGYRKNWLPGPMMKQQRFSKTASQNYKIPQGRN |
| | | NSLLHYETRTTLDGRWSNFAPGTAMATAANDATDFSQAQLIFAGPNITGN |
| | | TTTDANNLMFTSEDELRATNPRDTDLFGHLATNQQNATTVPTVDDVDGVG |
| | | VYPGMVWQDRDIYYQGPIWAKIPHTDGHFHPSPLIGGFGLKSPPPQIFIK |
| | | NTPVPANPATTFSPARINSFITQYSTGQVAVKIEWEIQKERSKRWNPEVQ |
| | | FTSNYGAQDSLLWAPDNAGAYKEPRAIGSRYLTNHL |
| 2 | AAVmo | MSFFDWLGKQYAQGAAEFWDLKSGPPAPKKARKDGSAGFNFPGHKYLGPG |
| | | NSLDRGDPVDADDAAAQKHDQSYQEQLEAGDNPYLKYNHADREFQEALKD |
| | | DTSFEGNLARGLFEAKKLVAEPLGLVEPELAPPSGRKRPVQSSQESGYSS |
| | | SQDKRPNLDVDEEDREFAAAAAETETGSAPPTGNLGPGTMAGGGSAPIDD |
| | | GSYGADGVGNASGDWHCDSTWLDNCVITRTTRTWNLPTYNNHIYKRLNGT |
| | | TSGDQSYFGFSTPWGYFDFNRFHCHFSPRDWQRLINNNWGLRPKSLRFKI |
| | | FNIQVKEVTTQDSTKIISNNLTSTVQVFADTEYQLPYVIGSAHEGCLPPF |
| | | PADVFMLPQYGYCTRQDGNSNNPTPRSAFYCLEYFPSKMLRTGNSFEFTY |
| | | NFEKVPFHSMWAHNQSLDRLMNPLIDQYLYYLDVTSSTGFTYQKGVHTNL |
| | | PEQERNWLPGPGIRNQAWFNSATGNNPLTGTWQYSNKYVLENRASKIAPG |
| | | PAMGIESTKFDGNGIIFSKEYITNVNTANPNQVNITRETEINSTNPLAGG |
| | | SLGAHANNSQNTTTAPTLDHTNVMGVFPGSVWQDRDIYLQGQIWAKIPHT |
| | | DGHFHPSPLMGGFGLKNPPPQILIKNTPVPADPPTEFNANKISSFITQYS |
| | | TGQVTVEMEWELQKETSKRWNPEIQYSDDSSSTSGSILHFAPDDVGNYKE |
| | | FRSIGTRYLTRPL |
| 3 | AAV2 | MAADGYLPDWLEDTLSEGIRQWWKLKPGPPPPKPAERHKDDSRGLVLPGY |
| | | KYLGPFNGLDKGEPVNEADAAALEHDKAYDRQLDSGDNPYLKYNHADAEF |
| | | QERLKEDTSFGGNLGRAVFQAKKRVLEPLGLVEEPVKTAPGKKRPVEHSP |
| | | VEPDSSSGTGKAGQQPARKRLNFGQTGDADSVPDPQPLGQPPAAPSGLGT |
| | | NTMATGSGAPMADNNEGADGVGNSSGNWHCDSTWMGDRVITTSTRTWALP |
| | | TYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQL |
| | | PYVLGSAHQGCLPPFPADVFMVPQYGYLTLNNGSQAVGRSSFYCLEYFPS |
| | | QMLRTGNNFTFSYTFEDVPFHSSYAHSQSLDRLMNPLIDQYLYYLSRTNT |
| | | PSGTTTQSRLQFSQAGASDIRDQSRNWLPGPCYRQQRVSKTSADNNNSEY |
| | | SWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGV |
| | | LPGMVWQDRDVYLQGPIWAKIPHTDGHFHPSPLMGGFGLKHPPPQILIKN |
| | | TPVPANPSTTFSAAKFASFITQYSTGQVSVEIEWELQKENSKRWNPEIQY |
| | | TSNYNKSVNVDFTVDTNGVYSEPRPIGTRYLTRNL |
| 4 | AAV4 | MTDGYLPDWLEDNLSEGVREWWALQPGAPKPKANQQHQDNARGLVLPGYK |
| | | YLGPGNGLDKGEPVNAADAAALEHDKAYDQQLKAGDNPYLKYNHADAEFQ |
| | | QRLQGDTSFGGNLGRAVFQAKKRVLEPLGLVEQAGETAPGKKRPLIESPQ |
| | | QPDSSTGIGKKGKQPAKKKLVFEDETGAGDGPPEGSTSGAMSDDSEMRAA |
| | | AGGAAVEGGQGADGVGNASGDWHCDSTWSEGHVTTTSTRTWVLPTYNNHL |
| | | YKRLGESLQSNTYNGFSTPWGYFDFNRFHCHFSPRDWQRLINNNWGMRPK |
| | | AMRVKIFNIQVKEVTTSNGETTVANNLTSTVQIFADSSYELPYVMDAGQE |
| | | GSLPPFPNDVFMVPQYGYCGLVTGNTSQQQTDRNAFYCLEYFPSQMLRTG |
| | | NNFEITYSFEKVPFHSMYAHSQSLDRLMNPLIDQYLWGLQSTTTGTTLNA |
| | | GTATTNFTKLRPTNFSNFKKNWLPGPSIKQQGFSKTANQNYKIPATGSDS |
| | | LIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAV |
| | | PGMVWQNRDIYYQGPIWAKIPHTDGHFHPSPLIGGFGLKHPPPQIFIKNT |
| | | PVPANPATTFSSTPVNSFITQYSTGQVSVQIDWEIQKERSKRWNPEVQFT |
| | | SNYGQQNSLLWAPDAAGKYTEPRAIGTRYLTHHL |
| 5 | AAV5 | MSFVDHPPDWLEEVGEGLREFLGLEAGPPKPKPNQQHQDQARGLVLPGYN |
| | | YLGPGNGLDRGEPVNRADEVAREHDISYNEQLEAGDNPYLKYNHADAEFQ |
| | | EKLADDTSFGGNLGKAVFQAKKRVLEPFGLVEEGAKTAPTGKRIDDHFPK |
| | | RKKARTEEDSKPSTSSDAEAGPSGSQQLQIPAQPASSLGADTMSAGGGGP |
| | | LGDNNQGADGVGNASGDWHCDSTWMGDRVVTKSTRTWVLPSYNNHQYREI |
| | | KSGSVDGSNANAYFGYSTPWGYFDFNRFHSHWSPRDWQRLINNYWGFRPR |
| | | SLRVKIFNIQVKEVTVQDSTTTIANNLTSTVQVFTDDDYQLPYVVGNGTE |
| | | GCLPAFPPQVFTLPQYGYATLNRDNTENPTERSSFFCLEYFPSKMLRTGN |
| | | NFEFTYNFEEVPFHSSFAPSQNLFKLANPLVDQYLYRFVSTNNTGGVQFN |
| | | KNLAGRYANTYKNWFPGPMGRTQGWNLGSGVNRASVSAFATTNRMELEGA |
| | | SYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERD |
| | | VYLQGPIWAKIPETGAHFHPSPAMGGFGLKHPPPMMLIKNTPVPGNITSF |
| | | SDVPVSSFITQYSTGQVTVEMEWELKKENSKRWNPEIQYTNNYNDPQFVD |
| | | FAPDSTGEYRTTRPIGTRYLTRPL |
| 6 | AAV6 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPFGLVEEGAKTAPGKKRPVEQSP |
| | | QEPDSSSGIGKTGQQPAKKRLNFGQTGDSESVPDPQPLGEPPATPAAVGP |
| | | TTMASGGGAPMADNNEGADGVGNASGNWHCDSTWLGDRVITTSTRTWALP |
| | | TYNNHLYKQISSASTGASNDNHYFGYSTPWGYFDFNRFHCHFSPRDWQRL |
| | | INNNWGFRPKRLNFKLFNIQVKEVTTNDGVTTIANNLTSTVQVFSDSEYQ |
| | | LPYVLGSAHQGCLPPFPADVFMIPQYGYLTLNNGSQAVGRSSFYCLEYFP |
| | | SQMLRTGNNFTFSYTFEDVPFHSSYAHSQSLDRLMNPLIDQYLYYLNRTQ |
| | | NQSGSAQNKDLLFSRGSPAGMSVQPKNWLPGPCYRQQRVSKTKTDNNNSN |
| | | FTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMG |
| | | ALPGMVWQDRDVYLQGPIWAKIPHTDGHFHPSPLMGGFGLKHPPPQILIK |
| | | NTPVPANPPAEFSATKFASFITQYSTGQVSVEIEWELQKENSKRWNPEVQ |
| | | YTSNYAKSANVDFTVDNNGLYTEPRPIGTRYLTRPL |
| 7 | AAV6.2 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPGKKRPVEQSP |
| | | QEPDSSSGIGKTGQQPAKKRLNFGQTGDSESVPDPQPLGEPPATPAAVGP |
| | | TTMASGGGAPMADNNEGADGVGNASGNWHCDSTWLGDRVITTSTRTWALP |
| | | TYNNHLYKQISSASTGASNDNHYFGYSTPWGYFDFNRFHCHFSPRDWQRL |
| | | INNNWGFRPKRLNFKLFNIQVKEVTTNDGVTTIANNLTSTVQVFSDSEYQ |
| | | LPYVLGSAHQGCLPPFPADVFMIPQYGYLTLNNGSQAVGRSSFYCLEYFP |
| | | SQMLRTGNNFTFSYTFEDVPFHSSYAHSQSLDRLMNPLIDQYLYYLNRTQ |
| | | NQSGSAQNKDLLFSRGSPAGMSVQPKNWLPGPCYRQQRVSKTKTDNNNSN |
| | | FTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMG |
| | | ALPGMVWQDRDVYLQGPIWAKIPHTDGHFHPSPLMGGFGLKHPPPQILIK |
| | | NTPVPANPPAEFSATKFASFITQYSTGQVSVEIEWELQKENSKRWNPEVQ |
| | | YTSNYAKSANVDFTVDNNGLYTEPRPIGTRYLTRPL |
| 8 | AAV7 | MAADGYLPDWLEDNLSEGIREWWDLKPGAPKPKANQQKQDNGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPAKKRPVEPSP |
| | | QRSPDSSTGIGKKGQQPARKRLNFGQTGDSESVPDPQPLGEPPAAPSSVG |
| | | SGTVAAGGGAPMADNNEGADGVGNASGNWHCDSTWLGDRVITTSTRTWAL |
| | | PTYNNHLYKQISSETAGSTNDNTYFGYSTPWGYFDFNRFHCHFSPRDWQR |
| | | LINNNWGFRPKKLRFKLFNIQVKEVTTNDGVTTIANNLTSTIQVFSDSEY |
| | | QLPYVLGSAHQGCLPPFPADVFMIPQYGYLTLNNGSQSVGRSSFYCLEYF |
| | | PSQMLRTGNNFEFSYSFEDVPFHSSYAHSQSLDRLMNPLIDQYLYYLART |
| | | QSNPGGTAGNRELQFYQGGPSTMAEQAKNWLPGPCFRQQRVSKTLDQNNN |
| | | SNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQ |
| | | GALPGMVWQNRDVYLQGPIWAKIPHTDGNFHPSPLMGGFGLKHPPPQILI |
| | | KNTPVPANPPEVFTPAKFASFITQYSTGQVSVEIEWELQKENSKRWNPEI |
| | | QYTSNFEKQTGVDFAVDSQGVYSEPRPIGTRYLTRNL |
| 9 | AAV8 | MAADGYLPDWLEDNLSEGIREWWALKPGAPKPKANQQKQDDGRGLVLPGY |
| | | KYLGPFNGLDKGEPVNAADAAALEHDKAYDQQLQAGDNPYLRYNHADAEF |
| | | QERLQEDTSFGGNLGRAVFQAKKRVLEPLGLVEEGAKTAPGKKRPVEPSP |
| | | QRSPDSSTGIGKKGQQPARKRLNFGQTGDSESVPDPQPLGEPPAAPSGVG |
| | | PNTMAAGGGAPMADNNEGADGVGSSSGNWHCDSTWLGDRVITTSTRTWAL |
| | | PTYNNHLYKQISNGTSGGATNDNTYFGYSTPWGYFDFNRFHCHFSPRDWQ |
| | | RLINNNWGFRPKRLSFKLFNIQVKEVTQNEGTKTIANNLTSTIQVFTDSE |
| | | YQLPYVLGSAHQGCLPPFPADVFMIPQYGYLTLNNGSQAVGRSSFYCLEY |
| | | FPSQMLRTGNNFQFTYTFEDVPFHSSYAHSQSLDRLMNPLIDQYLYYLSR |
| | | TQTTGGTANTQTLGFSQGGPNTMANQAKNWLPGPCYRQQRVSTTTGQNNN |
| | | SNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNS |
| | | QGALPGMVWQNRDVYLQGPIWAKIPHTDGNFHPSPLMGGFGLKHPPPQIL |
| | | IKNTPVPADPPTTFNQSKLNSFITQYSTGQVSVEIEWELQKENSKRWNPE |
| | | IQYTSNYYKSTSVDFAVNTEGVYSEPRPIGTRYLTRNL |
| 10 | AAV9 | MAADGYLPDWLEDNLSEGIREWWALKPGAPQPKANQQHQDNARGLVLPGY |
| | | KYLGPGNGLDKGEPVNAADAAALEHDKAYDQQLKAGDNPYLKYNHADAEF |
| | | QERLKEDTSFGGNLGRAVFQAKKRLLEPLGLVEEAAKTAPGKKRPVEQSP |
| | | QEPDSSAGIGKSGAQPAKKRLNFGQTGDTESVPDPQPIGEPPAAPSGVGS |
| | | LTMASGGGAPVADNNEGADGVGSSSGNWHCDSQWLGDRVITTSTRTWALP |
| | | TYNNHLYKQISNSTSGGSSNDNAYFGYSTPWGYFDFNRFHCHFSPRDWQR |
| | | LINNNWGFRPKRLNFKLFNIQVKEVTDNNGVKTIANNLTSTVQVFTDSDY |
| | | QLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKT |
| | | INGSGQNQQTLKFSVAGPSNMAVQGRNYIPGPSYRQQRVSTTVTQNNNSE |
| | | FAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQG |
| | | ILPGMVWQDRDVYLQGPIWAKIPHTDGNFHPSPLMGGFGMKHPPPQILIK |
| | | NTPVPADPPTAFNKDKLNSFITQYSTGQVSVEIEWELQKENSKRWNPEIQ |
| | | YTSNYYKSNNVEFAVNTEGVYSEPRPIGTRYLTRNL |
| 11 | AAVrh8 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPGKKRPVEQSP |
| | | QEPDSSSGIGKTGQQPAKKRLNFGQTGDSESVPDPQPLGEPPAAPSGLGP |
| | | NTMASGGGAPMADNNEGADGVGNSSGNWHCDSTWLGDRVITTSTRTWALP |
| | | TYNNHLYKQISNGTSGGSTNDNTYFGYSTPWGYFDFNRFHCHFSPRDWQR |
| | | LINNNWGFRPKRLNFKLFNIQVKEVTTNEGTKTIANNLTSTVQVFTDSEY |
| | | QLPYVLGSAHQGCLPPFPADVFMVPQYGYLTLNNGSQALGRSSFYCLEYF |
| | | PSQMLRTGNNFQFSYTFEDVPFHSSYAHSQSLDRLMNPLIDQYLYYLVRT |
| | | QTTGTGGTQTLAFSQAGPSSMANQARNWVPGPCYRQQRVSTTTNQNNNSN |
| | | FAWTGAAKFKLNGRDSLMNPGVAMASHKDDDDRFFPSSGVLIFGKQGAGN |
| | | DGVDYSQVLITDEEEIKATNPVATEEYGAVAINNQAANTQAQTGLVHNQG |
| | | VIPGMVWQNRDVYLQGPIWAKIPHTDGNFHPSPLMGGFGLKHPPPQILIK |
| | | NTPVPADPPLTFNQAKLNSFITQYSTGQVSVEIEWELQKENSKRWNPEIQ |
| | | YTSNYYKSTNVDFAVNTEGVYSEPRPIGTRYLTRNL |
| 12 | AAVrh 10 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPGKKRPVEPSP |
| | | QRSPDSSTGIGKKGQQPAKKRLNFGQTGDSESVPDPQPIGEPPAGPSGLG |
| | | SGTMAAGGGAPMADNNEGADGVGSSSGNWHCDSTWLGDRVITTSTRTWAL |
| | | PTYNNHLYKQISNGTSGGSTNDNTYFGYSTPWGYFDFNRFHCHFSPRDWQ |
| | | RLINNNWGFRPKRLNFKLFNIQVKEVTQNEGTKTIANNLTSTIQVFTDSE |
| | | YQLPYVLGSAHQGCLPPFPADVFMIPQYGYLTLNNGSQAVGRSSFYCLEY |
| | | FPSQMLRTGNNFEFSYQFEDVPFHSSYAHSQSLDRLMNPLIDQYLYYLSR |
| | | TQSTGGTAGTQQLLFSQAGPNNMSAQAKNWLPGPCYRQQRVSTTLSQNNN |
| | | SNFAWTGATKYHLNGRDSLVNPGVAMATHKDDEERFFPSSGVLMFGKQGA |
| | | GKDNVDYSSVMLTSEEEIKTTNPVATEQYGVVADNLQQQNAAPIVGAVNS |
| | | QGALPGMVWQNRDVYLQGPIWAKIPHTDGNFHPSPLMGGFGLKHPPPQIL |
| | | IKNTPVPADPPTTFSQAKLASFITQYSTGQVSVEIEWELQKENSKRWNPE |
| | | IQYTSNYYKSTNVDFAVNTDGTYSEPRPIGTRYLTRNL |
| 13 | AAVanc80 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPGKKRPVEQSP |
| | | QEPDSSSGIGKKGQQPARKRLNFGQTGDSESVPDPQPLGEPPAAPSGVGS |
| | | NTMAAGGGAPMADNNEGADGVGNASGNWHCDSTWLGDRVITTSTRTWALP |
| | | TYNNHLYKQISSQSGGSTNDNTYFGYSTPWGYFDFNRFHCHFSPRDWQRL |
| | | INNNWGFRPKKLNFKLFNIQVKEVTTNDGTTTIANNLTSTVQVFTDSEYQ |
| | | LPYVLGSAHQGCLPPFPADVFMIPQYGYLTLNNGSQAVGRSSFYCLEYFP |
| | | SQMLRTGNNFQFSYTFEDVPFHSSYAHSQSLDRLMNPLIDQYLYYLSRTQ |
| | | TTSGTAGNRTLQFSQAGPSSMANQAKNWLPGPCYRQQRVSKTTNQNNNSN |
| | | FAWTGATKYHLNGRDSLVNPGPAMATHKDDEDKFFPMSGVLIFGKQGAGN |
| | | SNVDLDNVMITNEEEIKTTNPVATEEYGTVATNLQSANTAPATGTVNSQG |
| | | ALPGMVWQDRDVYLQGPIWAKIPHTDGHFHPSPLMGGFGLKHPPPQILIK |
| | | NTPVPANPPTTFSPAKFASFITQYSTGQVSVEIEWELQKENSKRWNPEIQ |
| | | YTSNYNKSTNVDFAVDTNGVYSEPRPIGTRYLTRNL |
| 14 | AAVanc110 | MAADGYLPDWLEDNLSEGIREWWDLKPGAPKPKANQQKQDDGRGLVLPGY |
| | | KYLGPFNGLDKGEPVNAADAAALEHDKAYDQQLKAGDNPYLRYNHADAEF |
| | | QERLQEDTSFGGNLGRAVFQAKKRVLEPLGLVEEGAKTAPGKKRPVEQSP |
| | | QEPDSSSGIGKTGQQPAKKRLNFGQTGDSESVPDPQPLGEPPAAPSGVGS |
| | | NTMASGGGAPMADNNEGADGVGNSSGNWHCDSTWLGDRVITTSTRTWALP |
| | | TYNNHLYKQISNGTSGGSTNDNTYFGYSTPWGYFDFNRFHCHFSPRDWQR |
| | | LINNNWGFRPKRLNFKLFNIQVKEVTTNEGTKTIANNLTSTVQVFTDSEY |
| | | QLPYVLGSAHQGCLPPFPADVFMIPQYGYLTLNNGSQAVGRSSFYCLEYF |
| | | PSQMLRTGNNFQFSYTFEDVPFHSSYAHSQSLDRLMNPLIDQYLYYLSRT |
| | | QTTGTAGTQTLQFSQAGPSSMANQARNWVPGPCYRQQRVSTTTNQNNNSN |
| | | FAWTGATKYHLNGRDSLMNPGVAMASHKDDEDRFFPSSGVLIFGKQGAGN |
| | | DNVDYSQVMITNEEEIKTTNPVATEEYGAVATNNQSANTQAQTGLVHNQG |
| | | VLPGMVWQNRDVYLQGPIWAKIPHTDGNFHPSPLMGGFGLKHPPPQILIK |
| | | NTPVPADPPTTFNQAKLNSFITQYSTGQVSVEIEWELQKENSKRWNPEIQ |
| | | YTSNYYKSTNVDFAVNTEGVYSEPRPIGTRYLTRNL |
| 173 | LK03 | MAADGYLPDWLEDNLSEGIREWWALQPGAPKPKANQQHQDNARGLVLPGY |
| | | KYLGPGNGLDKGEPVNAADAAALEHDKAYDQQLKAGDNPYLKYNHADAEF |
| | | QERLKEDTSFGGNLGRAVFQAKKRLLEPLGLVEEAAKTAPGKKRPVDQSP |
| | | QEPDSSSGVGKSGKQPARKRLNFGQTGDSESVPDPQPLGEPPAAPTSLGS |
| | | NTMASGGGAPMADNNEGADGVGNSSGNWHCDSQWLGDRVITTSTRTWALP |
| | | TYNNHLYKQISSQSGASNDNHYFGYSTPWGYFDFNRFHCHFSPRDWQRLI |
| | | NNNWGFRPKKLSFKLFNIQVKEVTQNDGTTTIANNLTSTVQVFTDSEYQL |
| | | PYVLGSAHQGCLPPFPADVFMVPQYGYLTLNNGSQAVGRSSFYCLEYFPS |
| | | QMLRTGNNFQFSYTFEDVPFHSSYAHSQSLDRLMNPLIDQYLYYLNRTQG |
| | | TTSGTTNQSRLLFSQAGPQSMSLQARNWLPGPCYRQQRLSKTANDNNNSN |
| | | FPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTRTVNDQG |
| | | ALPGMVWQDRDVYLQGPIWAKIPHTDGHFHPSPLMGGFGLKHPPPQIMIK |
| | | NTPVPANPPTTFSPAKFASFITQYSTGQVSVEIEWELQKENSKRWNPEIQ |
| | | YTSNYNKSVNVDFTVDTNGVYSEPRPIGTRYLTRPL |
| 165 | Bba.45 | atggctgctgacggttatcttccagattggctcgaggacaacctctctga |
| | | aggcattcgcgagtggtgggcgctgaaacctggagccccacagcccaagg |
| | | caaatcgacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttc |
| | | cagcagcgcttggcgaccgacacctcttttgggggcaacctcggcaaggc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaagggcactcgactttgaag |
| | | actctggagcaggagacggaccccctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcagggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggaacagctaccaccacgtacgggaa |
| | | aattaccactggggactttgcctactacaggaaaaactggttacctggag |
| | | cctgcattaaacaacaaaaattttcaaagaatgccagtcaaaactacaag |
| | | attcccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccaccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 166 | Bba.46 | atggctgctgacggttatcttccagattggctcgaggacaacctctctga |
| | | aggcattcgcgagtggtgggcgctgaaacctggagccccacagcccaagg |
| | | caaatcaacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttc |
| | | cagcagcgcttggcgaccgacacctcttttgggggcaacctcggcaaggc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggaccccctgagggatcatcttccggagaaatg |
| | | tctcatgacgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatgctttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggagcagctaccaccacgtacgggaa |
| | | aattaccactggggactttgcctactacaggaaaaactggttgcctggag |
| | | cctgcattaaacaacaaaaattttcaaagaatgccagtcaaaactacaag |
| | | atccccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccaccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 167 | Bba.47 | atggctgctgacggttatcttccagattggctcgaggacaacctctctga |
| | | aggcattcgcgagtggtgggcgctgaaacctggagccccacagcccaagg |
| | | caaatcaacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttc |
| | | cagcagcgcttggcgaccgacacctcttttgggggcaacctcggcaaggc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggacctcctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggaacagctaccaccacgtacgggaa |
| | | aattaccactggggactttgcctactacaggaaaaactggttgcctggag |
| | | cctgcattaaacaacaaaaattttcaaagaatgccagtcaaaactacaag |
| | | attcccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | ggcagattgcagataataatcaaaatgccaccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 168 | Bba.48 | atggctgctgacggttatcttccagattggctcgaggacaacctctctga |
| | | aggcattcgcgagtggtgggcgctgaaacctggagccccacagcccaagg |
| | | caaatcaacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttt |
| | | caggagcgtcttcaagaagatacgtcttttgggggcaacctcgggcgagc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggaccccctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccgcttttccccgcgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggaacagctatcaccacgtacgggaa |
| | | aattaccactggggactttgcctactacaggaaaaactggttgcctggag |
| | | cctgcattaaacaacaaaaattttcaaagaatgccagtcaaaactacaag |
| | | attcccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccgccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 169 | Bba.49 | atggctgctgacggttatcttccagattggctcgaggacaacctctctga |
| | | aggcattcgcgagtggtgggcgctgaaacctggagccccacagcccaagg |
| | | caaatcaacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttt |
| | | caggagcgtcttcaagaagatacgtcttttgggggcaacctcgggcgagc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggaccccctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggaacagctatcaccacgtacgggaa |
| | | aattaccactggggactttgcctactacaggaaaaactggttgcctggag |
| | | ccggcattaaacaacaaaaattttcaaagaatgccagtcaaaactacaag |
| | | attcccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccaccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 170 | Bba.50 | atggctgctgacggttatcttccagattggctcgaggacaacctctctga |
| | | aagcattcgcgagtggtgggcgctgaaacctggagccccacggcccaagg |
| | | caaatcaacaacatcaagacgacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttc |
| | | cagcagcgcttggcgaccgacacctcttttgggggcaacctcggcaaggc |
| | | agtcttccaggccaaaaagaggattctcgagcctctgggtctggttgaag |
| | | agggcgttaaaacggctcctggaaggaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggaccccctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctctccattcaatgtacgcgcacagccagagcctggaca |
| | | gaatgatgaatcctttgctggatcagtacctgtggcatctgcaatcgacc |
| | | actaccggaaattcccttaatcaaggaacagctaccaccacgtacgggaa |
| | | aattaccactggggactttgcctactacaggaaaaactggttgcctggag |
| | | cctgcattaaacaacaaaaattttcaaagaatgccagtcaaaactacaag |
| | | attcccgccagcggggaagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccaccaccgcccctcacatcgct |
| | | aacctggacgctatgggaattgttcccggaatggtctggcaaaacagaga |
| | | catctactaccagggccctatctgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |
| 171 | Bba.51 | atggctgctgacggttatcttccagattggctcgaggacaacctctctga |
| | | aggcattcgcgagtggtgggcgctgaaacctggagccccacagcccaagg |
| | | caaatcaacaacatcaagacaacgctcggggtcttgtgcttccgggttac |
| | | aaatacttgggacccggtaacggactcgacaagggagagccggtcaacga |
| | | ggcagacgccgcggccctcgagcacgacaaggcctacgaccagcagctca |
| | | agtcgggagacaacccgtacctcaagtacaaccacgcggacgccgagttc |
| | | cagcagcgcttggcgaccgacacctcttttgggggcaacctcggcaaggc |
| | | agtcttccaggccaaaaagaggattctcgagcctctgggtctggttgaag |
| | | agggcgttaaaacggctcctggaaagaaacgcccattagaaaagactcca |
| | | aatcggccgaccaacccggactctgggaaggccccggccaagaaaaagca |
| | | aaaagacggcgagacagccgactctgctagaaggacactcgactttgaag |
| | | actctggagcaggagacggaccccctgagggatcatcttccggagaaatg |
| | | tctcatgatgctgagatgcgtgcggcgccaggcggaaatgctgtcgaggc |
| | | gggacaaggtgccgatggagtgggtaatgcctccggtgattggcattgcg |
| | | attccacctggtcagagggccgagtcaccaccaccagcacccgaacctgg |
| | | gtcctgcccacctacaacaaccacctgtacctgcgaatcggaacaacggc |
| | | caacagcaacacctacaatggattctccaccccctggggatactttgact |
| | | ttaaccgcttccactgccacttttccccacgcgactggcagcgactcatc |
| | | aacaacaactggggactcaggccgaaatcgatgcgtgttaaaatcttcaa |
| | | catccaggtcaaggaggtcactacgtcaaacggcgagactacggtcgcta |
| | | ataaccttaccagcacggttcagatctttgcggattcaacgtatgaactc |
| | | ccatacgtgatggacgccggtcaggaggggagccttcctccgttccccaa |
| | | cgacgtgtttatggttccccaatacgggtactgcggagtcgtcactggag |
| | | aaaaccagaaccaaacagacagaaatgccttttactgtctggagtacttt |
| | | ccatcccaaatgctaagaactggcaacaactttgaaatcagttaccaatt |
| | | tgaaaaagttcctttccattcaatgtacgcgcacagccagagcctggaca |
| | | gaatgatgaatcctttgctggatcagtacctgtggcatctgcaatcgacc |
| | | actaccggaaattcccttaatcaaggaacagctaccaccacgtacgggaa |
| | | aattaccactggggactttgcctactacaggaaaaactggttgcctggag |
| | | cctgcattaaacaacaaaaattttcaaagaatgccagtcaaaactacaag |
| | | attcccgccagcgggggagacgcccttttaaagtatgacacgcataccac |
| | | tttaaatgggcgatggagtaacatggctcctggtcctccaatggccaccg |
| | | caggtgccggggactcggattttagcaacagccagctgatctttgccgga |
| | | cccaatcagagcggtaacacgaccacgtcttcaaacaatttgttgtttac |
| | | ctcagaagaggagattgccacaacaaacccacgagacacggacatgtttg |
| | | gacagattgcagataataatcaaaatgccaccaccgcccctcacatcgct |
| | | aacctggacgctatgggaattgttcccggaatggtctggcaaaacagaga |
| | | catctactaccagggccctatttgggccaaggtccctcacacggacggac |
| | | actttcacccttcgccgctgatgggaggatttggactgaaacacccgcct |
| | | ccgcagattttcatcaaaaacacccccgtacccgccaatcccaatactac |
| | | ctttagcgctgcaaggatcaattcttttttgacgcagtacagcaccggac |
| | | aagtcgccgttcagatcgactgggaaattcagaaggagcactccaaacgc |
| | | tggaatcccgaagtccaatttacttcaaactacggcactcaaaattctat |
| | | gctgtgggctcccgacaacgccggcaactaccacgaaccccgggctattg |
| | | ggtcccgtttcctcacccaccacttgtaa |

TABLE 7
Chimeric AAV Capsid Proteins

| SEQ ID | |
|---|---|
| NO. | Sequence |
| 90 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEPSPQRSPDSSTGIGKKGQQPAKKRLNFGQTGDSESV |
| | PDPQPIGEPPAGPSGLGSGTMAAGGGAPMADNNEGADGVGSSSGNWHCDSTWLGDRVITTS |
| | TRTWALPTYNNHLYKQISNGTSGGSTNDNTYFGYSTPWGYFDFNRFHCHFSPRDWQRLINN |
| | NWGFRPKRLNFKLFNIQVKEVTQNEGTKTIANNLTSTIQVFTDSEYQLPYVLGSAHQGCLP |
| | PFPADVFMIPQYGYLTLNNGSQAVGRSSFYCLEYFPSQMLRTGNNFEFSYQFEDVPFHSSY |
| | AHSQSLDRLMNPLIDQYLYYLSKTINGSGQNQQTLKFSVAGPSNMAVQGRNYIPGPSYRQQ |
| | RVSTTVTQNNNSEFAWPGASSWALNGRNSLMNPGPAMASHKEGEDRFFPLSGSLIFGKQGT |
| | GRDNVDADKVMITNEEEIKTTNPVATESYGQVATNHQSAQAQAQTGWVQNQGILPGMVWQN |
| | RDVYLQGPIWAKIPHTDGNFHPSPLMGGFGLKHPPPQILIKNTPVPADPPTTFSQAKLASF |
| | ITQYSTGQVSVEIEWELQKENSKRWNPEIQYTSNYYKSTNVDFAVNTDGTYSEPRPIGTRY |
| | LTRNL |
| 91 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEPSPQRSPDSSTGIGKKGQQPAKKRLNFGQTGDSESV |
| | PDPQPIGEPPAGPSGLGSGTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLNFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFEFSYQFEDVPFHSSY |
| | AHSQSLDRLMNPLIDQYLYYLSRTINGSGQNQQTLKFSVAGPSNMAVQAKNWLPGPCYRQQ |
| | RVSTTVTQNNNSEFAWPGATKYHLNGRDSLVNPGVAMASHKEGEDRFFPLSGSLMFGKQGT |
| | GRDNVDADKVMLTSEEEIKTTNPVATEQYGVVATNHQSAQAQAQTGWVQNQGILPGMVWQN |
| | RDVYLQGPIWAKIPHTDGNFHPSPLMGGFGLKHPPPQILIKNTPVPADPPTTFSQAKLASF |
| | ITQYSTGQVSVEIEWELQKENSKRWNPEIQYTSNYYKSNNVEFAVNTEGTYSEPRPIGTRY |
| | LTRNL |
| 92 | MAADGYLPDWLEDNLSEGIREWWALKPGAPQPKANQQKQDDGRGLVLPGYKYLGPFNGLDK |
| | GEPVNEADAAALEHDKAYDQQLKAGDNPYLRYNHADAEFQERLQEDTSFGGNLGRAVFQAK |
| | KRVLEPLGLVEEAAKTAPGKKRPVDSPDSTSGIGKKGQQPARKRLNFGQTGDAESVPDPQP |
| | IGEPPAAPSGLGSGTMAAGGGAPMADNNEGADGVGNASGNWHCDSTWLGNRVITTSTRTWA |
| | LPTYNNHLYKQISSSSSGATNDNHYFGYSTPWGYFDFNRFHCHFSPRDWQRLINNNWGFRP |
| | KRLRFKLFNIQVKEVTTNDGVTTIANNLTSTVQVFSDSEYQLPYVLGSAHQGCLPPFPADV |
| | FMIPQYGYLTLNNGSQSVGRSSFYCLEYFPSQMLRTGNNFEFSYEFEDVPFHSSYAHSQSL |
| | DRLMNPLIDQYLYYLSKTINGSGQNQQTLKFSVAGPSNMAVQGRNYIPGPSYRQQRVSTTV |
| | TQNNNSEFAWPGASSWALNGRNSLMNPGPAMASHKEGEDRFFPLSGSLIFGKQGTGRDNVD |
| | ADKVMITNEEEIKTTNPVATESYGQVATNHQSAQAQAQTGWVQNQGILPGMVWQNRDVYLQ |
| | GPIWAKIPHTDGNFHPSPLMGGFGLKHPPPQILIKNTPVPANPPETFTPAKFASFITQYST |
| | GQVSVEIEWELQKENSKRWNPEIQYTSNYDKQTGVDFAVDTQGVYSEPRPIGTRYLTRNL |
| 93 | MAADGYLPDWLEDNLSEGIREWWALKPGAPQPKANQQKQDDGRGLVLPGYKYLGPFNGLDK |
| | GEPVNEADAAALEHDKAYDQQLKAGDNPYLRYNHADAEFQERLQEDTSFGGNLGRAVFQAK |
| | KRVLEPLGLVEEAAKTAPGKKRPVDSPDSTSGIGKKGQQPARKRLNFGQTGDAESVPDPQP |
| | IGEPPAAPSGLGSGTMAAGGGAPMADNNEGADGVGNASGNWHCDSTWLGNRVITTSTRTWA |
| | LPTYNNHLYKQISNSTSGGSSNDNHYFGYSTPWGYFDFNRFHCHFSPRDWQRLINNNWGFR |
| | PKRLRFKLFNIQVKEVTDNNGVKTIANNLTSTVQVFSDSEYQLPYVLGSAHQGCLPPFPAD |
| | VFMIPQYGYLTLNDGSQAVGRSSFYCLEYFPSQMLRTGNNFEFSYEFEDVPFHSSYAHSQS |
| | LDRLMNPLIDQYLYYLARTINGSGQNQQTLKFSVAGPSNMAVQSKNWLPGPCFRQQRVSTT |
| | VTQNNNSEFAWPGATKYHLNGRNSLTNPGVPMASHKEGEDRFFPLSGSLVFGKQGTGRDNV |
| | DADKVLMTDEEEIKATNPVATEEYGVVATNHQSAQAQAQTGWVQNQGILPGMVWQNRDVYL |
| | QGPIWAKIPHTDGNFHPSPLMGGFGLKHPPPQILIKNTPVPANPPETFTPAKFASFITQYS |
| | TGQVSVEIEWELQKENSKRWNPEIQYTSNYYKSNNVEFAVNTEGVYSEPRPIGTRYLTRNL |
| 94 | MAADGYLPDWLEDNLSEGIREWWDLKPGAPKPKANQQKQDNGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPAKKRPVEPSPQRSPDSSTGIGKKGQQPARKRLNFGQTGDSESV |
| | PDPQPLGEPPAAPSSVGSGTVAAGGGAPMADNNEGADGVGNASGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKKLRFKLFNIQVKEVTDNNGVKTIANNLTSTIQVFSDSEYQLPYVLGSAHQGCLP |
| | PFPADVFMIPQYGYLTLNDGSQAVGRSSFYCLEYFPSQMLRTGNNFEFSYSFEDVPFHSSY |
| | AHSQSLDRLMNPLIDQYLYYLARTINGSGQNQQTLKFSVAGPSNMAVQAKNWLPGPCFRQQ |
| | RVSTTVTQNNNSEFAWPGATKYHLNGRNSLVNPGVAMASHKEGEDRFFPLSGSLIFGKQGT |
| | GRDNVDADKLMTNEEEIRPTNPVATEEYGIVATNHQSAQAQAQTGWVQNQGILPGMVWQNR |
| | DVYLQGPIWAKIPHTDGNFHPSPLMGGFGLKHPPPQILIKNTPVPANPPEVFTPAKFASFI |
| | TQYSTGQVSVEIEWELQKENSKRWNPEIQYTSNFYKSNNVEFAVNTEGVYSEPRPIGTRYL |
| | TRNL |
| 95 | MAADGYLPDWLEDNLSEGIREWWDLKPGAPKPKANQQKQDNGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPAKKRPVEPSPQRSPDSSTGIGKKGQQPARKRLNFGQTGDSESV |
| | PDPQPLGEPPAAPSSVGSGTVAAGGGAPMADNNEGADGVGNASGNWHCDSTWLGDRVITTS |
| | TRTWALPTYNNHLYKQISSETAGSTNDNTYFGYSTPWGYFDFNRFHCHFSPRDWQRLINNN |
| | WGFRPKKLRFKLFNIQVKEVTTNDGVTTIANNLTSTIQVFSDSEYQLPYVLGSAHQGCLPP |
| | FPADVFMIPQYGYLTLNNGSQSVGRSSFYCLEYFPSQMLRTGNNFEFSYSFEDVPFHSSYA |
| | HSQSLDRLMNPLIDQYLYYLSKTINGSGQNQQTLKFSVAGPSNMAVQGRNYIPGPSYRQQR |
| | VSTTVTQNNNSEFAWPGASSWALNGRNSLMNPGPAMASHKEGEDRFFPLSGSLIFGKQGTG |
| | RDNVDADKVMITNEEEIKTTNPVATESYGQVATNHQSAQAQAQTGWVQNQGILPGMVWQNR |
| | DVYLQGPIWAKIPHTDGNFHPSPLMGGFGLKHPPPQILIKNTPVPANPPEVFTPAKFASFI |
| | TQYSTGQVSVEIEWELQKENSKRWNPEIQYTSNFEKQTGVDFAVDSQGVYSEPRPIGTRYL |
| | TRNL |
| 96 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPATPAAVGPTTMASGGGAPMADNNEGADGVGNASGNWHCDSTWLGDRVITTST |
| | RTWALPTYNNHLYKQISNSTSGGSSNDNHYFGYSTPWGYFDFNRFHCHFSPRDWQRLINNN |
| | WGFRPKRLNFKLFNIQVKEVTDNNGVKTIANNLTSTVQVFSDSEYQLPYVLGSAHQGCLPP |
| | FPADVFMIPQYGYLTLNDGSQAVGRSSFYCLEYFPSQMLRTGNNFTFSYTFEDVPFHSSYA |
| | HSQSLDRLMNPLIDQYLYYLNRTINGSGQNQQTLKFSVAGPSNMAVQPKNWLPGPCYRQQR |
| | VSTTVTQNNNSEFAWPGASKYNLNGRESIINPGTAMASHKEGEDRFFPLSGSMIFGKQGTG |
| | RDNVDADKVMITDEEEIKATNPVATERFGTVATNHQSAQAQAQTGWVQNQGILPGMVWQDR |
| | DVYLQGPIWAKIPHTDGHFHPSPLMGGFGLKHPPPQILIKNTPVPANPPAEFSATKFASFI |
| | TQYSTGQVSVEIEWELQKENSKRWNPEVQYTSNYYKSNNVEFAVNTEGLYTEPRPIGTRYL |
| | TRPL |
| 97 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPATPAAVGPTTMASGGGAPMADNNEGADGVGNASGNWHCDSTWLGDRVITTST |
| | RTWALPTYNNHLYKQISSASTGASNDNHYFGYSTPWGYFDFNRFHCHFSPRDWQRLINNNW |
| | GFRPKRLNFKLFNIQVKEVTTNDGVTTIANNLTSTVQVFSDSEYQLPYVLGSAHQGCLPPF |
| | PADVFMIPQYGYLTLNNGSQAVGRSSFYCLEYFPSQMLRTGNNFTFSYTFEDVPFHSSYAH |
| | SQSLDRLMNPLIDQYLYYLSKTINGSGQNQQTLKFSVAGPSNMAVQGRNYIPGPSYRQQRV |
| | STTVTQNNNSEFAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQGILPGMVWQDRD |
| | VYLQGPIWAKIPHTDGHFHPSPLMGGFGLKHPPPQILIKNTPVPANPPAEFSATKFASFIT |
| | QYSTGQVSVEIEWELQKENSKRWNPEVQYTSNYAKSANVDFTVDNNGLYTEPRPIGTRYLT |
| | RPL |
| 98 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KRLSSQSGASNDNHSYFGFSTPWGYFDFNRFHCHFSPRDWQRLINNNWGLRPKSLRFKIFN |
| | IQVKEVTQNDGTTTISNNLTSTVQVFADTEYQLPYVIGSAHEGCLPPFPADVFMLPQYGYC |
| | TLNNGSQPTPRSAFYCLEYFPSKMLRTGNSFEFTYNFEKVPFHSMWAHNQSLDRLMNPLID |
| | QYLYYLDVTNTPSGTTTQSRFTYQKGVHTNLPEQERNWLPGPGIRNQRVSKTSADNNNSEY |
| | SWQYSNKYVLENGRASKIAPGPAMASHKDDEEKFFPQSGVLIFGKQGSEKTNVDIEKVMIT |
| | RETEINSTNPLAGGSLGAVSTNLQRGNRQAALDHTNVMGVFPGSVWQDRDIYLQGQIWAKI |
| | PHTDGHFHPSPLMGGFGLKNPPPQILIKNTPVPADPPTEFNANKISSFITQYSTGQVTVEM |
| | EWELQKETSKRWNPEIQYTSNYNKSVNVHFAPDDVGNYKEFRSIGTRYLTRPL |
| 99 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KRLSSQSGASNDNHSYFGFSTPWGYFDFNRFHCHFSPRDWQRLINNNWGLRPKSLRFKIFN |
| | IQVKEVTQNDGTTTISNNLTSTVQVFADTEYQLPYVIGSAHEGCLPPFPADVFMLPQYGYC |
| | TLNNGSQPTPRSAFYCLEYFPSKMLRTGNSFEFTYNFEDVPFHSSYAHSQSLDRLMNPLID |
| | QYLYYLSRTNTPSGTTTQSRLQFSQAGASDIRDQSRNWLPGPCYRQQRVSKTSADNNNSEY |
| | SWTGATKYHLNGRDSLVNPGPAMASHKDDEEKFFPQSGVLIFGKQGSEKTNVDIEKVMITD |
| | EEEIRTTNPVATEQYGSVSTNLQRGNRQAATADVNTQGVFPGSVWQDRDIYLQGQIWAKIP |
| | HTDGHFHPSPLMGGFGLKNPPPQILIKNTPVPADPPTEFNANKISSFITQYSTGQVTVEME |
| | WELQKETSKRWNPEIQYTSNYNKSVNVHFAPDDVGNYKEFRSIGTRYLTRPL |
| 100 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KQISNSTSGGSSNDNAYFGFSTPWGYFDFNRFHCHFSPRDWQRLINNNWGLRPKSLRFKIF |
| | NIQVKEVTDNNGVKIISNNLTSTVQVFADTEYQLPYVIGSAHEGCLPPFPADVFMLPQYGY |
| | LTLNDGSQAVGRSSFYCLEYFPSKMLRTGNSFEFTYNFEKVPFHSMWAHNQSLDRLMNPLI |
| | DQYLYYLSKTINGSGQNQQTLKFSVAGPSNMAVQGRNWLPGPGIRQQRVSTTVTQNNNSEF |
| | AWPGASSWALNGRASKIAPGPAMASHKEGEDRFFPLSGSLIFGKQGTGRDNVDADKVNITR |
| | ETEINSTNPLAGGSLGQVATNHQSAQAQAQTGWVQNQGVFPGSVWQDRDIYLQGQIWAKIP |
| | HTDGHFHPSPLMGGFGLKNPPPQILIKNTPVPADPPTEFNANKISSFITQYSTGQVTVEME |
| | WELQKETSKRWNPEIQYTSNYYKSNNVEFAVNTEGVYKEFRSIGTRYLTRPL |
| 101 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KRLNGTTSGDQSYFGFSTPWGYFDFNRFHCHFSPRDWQRLINNNWGLRPKSLRFKIFNIQV |
| | KEVTTQDSTKIISNNLTSTVQVFADTEYQLPYVIGSAHEGCLPPFPADVFMLPQYGYCTRQ |
| | DGNSNNPTPRSAFYCLEYFPSKMLRTGNSFEFTYNFEKVPFHSMWAHNQSLDRLMNPLIDQ |
| | YLYYLSKTINGSGQNQQTLKFSVAGPSNMAVQGRNYIPGPSYRQQRVSTTVTQNNNSEFAW |
| | PGASSWALNGRNSLMNPGPAMASHKEGEDRFFPLSGSLIFGKQGTGRDNVDADKVMITNEE |
| | EIKTTNPVATESYGQVATNHQSAQAQAQTGWVQNQGVFPGSVWQDRDIYLQGQIWAKIPHT |
| | DGHFHPSPLMGGFGLKNPPPQILIKNTPVPADPPTEFNANKISSFITQYSTGQVTVEMEWE |
| | LQKETSKRWNPEIQYSDDSSSTSGSILHFAPDDVGNYKEFRSIGTRYLTRPL |
| 102 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KQISNSTSGGSSNDNAYFGFSTPWGYFDFNRFHCHFSPRDWQRLINNNWGLRPKSLRFKIF |
| | NIQVKEVTDNNGVKIISNNLTSTVQVFADTEYQLPYVIGSAHEGCLPPFPADVFMLPQYGY |
| | LTLNDGSQAVGRSSFYCLEYFPSKMLRTGNSFEFTYNFEKVPFHSMWAHNQSLDRLMNPLI |
| | DQYLYYLSKTINGSGQNQQTLKFSVAGPSNMAVQGRNYIPGPSYRQQRVSTTVTQNNNSEF |
| | AWPGASSWALNGRNSLMNPGPAMASHKEGEDRFFPLSGSLIFGKQGTGRDNVDADKVMITN |
| | EEEIKTTNPVATESYGQVATNHQSAQAQAQTGWVQNQGVFPGSVWQDRDIYLQGQIWAKIP |
| | HTDGHFHPSPLMGGFGLKNPPPQILIKNTPVPADPPTEFNANKISSFITQYSTGQVTVEME |
| | WELQKETSKRWNPEIQYTSNYYKSNNVEFAVNTEGVYKEFRSIGTRYLTRPL |
| 103 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KQISNGTSGGATNDNTYFGFSTPWGYFDFNRFHCHFSPRDWQRLINNNWGLRPKSLRFKIF |
| | NIQVKEVTQNEGTKIISNNLTSTVQVFADTEYQLPYVIGSAHEGCLPPFPADVFMLPQYGY |
| | LTLNNGSQAVGRSSFYCLEYFPSKMLRTGNSFEFTYNFEKVPFHSMWAHNQSLDRLMNPLI |
| | DQYLYYLSRTQTTGGTANTQTLGFSQGGPNTMANQAKNWLPGPGIRQQRVSTTTGQNNNSN |
| | FAWTAGTKYHLNGRASKIAPGPAMATHKDDEERFFPSNGILIFGKQNAARDNADYSDVNIT |
| | RETEINSTNPLAGGSLGIVADNLQQQNTAPQIGTVNSQGVFPGSVWQDRDIYLQGQIWAKI |
| | PHTDGHFHPSPLMGGFGLKNPPPQILIKNTPVPADPPTEFNANKISSFITQYSTGQVTVEM |
| | EWELQKETSKRWNPEIQYTSNYYKSTSVDFAVNTEGVYKEFRSIGTRYLTRPL |
| 104 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KRLNGTTSGDQSYFGFSTPWGYFDFNRFHCHFSPRDWQRLINNNWGLRPKSLRFKIFNIQV |
| | KEVTTQDSTKIISNNLTSTVQVFADTEYQLPYVIGSAHEGCLPPFPADVFMLPQYGYCTRQ |
| | DGNSNNPTPRSAFYCLEYFPSKMLRTGNSFEFTYNFEKVPFHSMWAHNQSLDRLMNPLIDQ |
| | YLYYLSRTQTTGGTANTQTLGFSQGGPNTMANQAKNWLPGPCYRQQRVSTTTGQNNNSNFA |
| | WTAGTKYHLNGRNSLANPGIAMATHKDDEERFFPSNGILIFGKQNAARDNADYSDVMLTSE |
| | EEIKTTNPVATEEYGIVADNLQQQNTAPQIGTVNSQGVFPGSVWQDRDIYLQGQIWAKIPH |
| | TDGHFHPSPLMGGFGLKNPPPQILIKNTPVPADPPTEFNANKISSFITQYSTGQVTVEMEW |
| | ELQKETSKRWNPEIQYSDDSSSTSGSILHFAPDDVGNYKEFRSIGTRYLTRPL |
| 105 | MSFFDWLGKQYAQGAAEFWDLKSGPPAPKKARKDGSAGFNFPGHKYLGPGNSLDRGDPVDA |
| | DDAAAQKHDQSYQEQLEAGDNPYLKYNHADREFQEALKDDTSFEGNLARGLFEAKKLVAEP |
| | LGLVEPELAPPSGRKRPVQSSQESGYSSSQDKRPNLDVDEEDREFAAAAAETETGSAPPTG |
| | NLGPGTMAGGGSAPIDDGSYGADGVGNASGDWHCDSTWLDNCVITRTTRTWNLPTYNNHIY |
| | KQISNGTSGGATNDNTYFGFSTPWGYFDFNRFHCHFSPRDWQRLINNNWGLRPKSLRFKIF |
| | NIQVKEVTQNEGTKIISNNLTSTVQVFADTEYQLPYVIGSAHEGCLPPFPADVFMLPQYGY |
| | LTLNNGSQAVGRSSFYCLEYFPSKMLRTGNSFEFTYNFEKVPFHSMWAHNQSLDRLMNPLI |
| | DQYLYYLSRTQTTGGTANTQTLGFSQGGPNTMANQAKNWLPGPCYRQQRVSTTTGQNNNSN |
| | FAWTAGTKYHLNGRNSLANPGIAMATHKDDEERFFPSNGILIFGKQNAARDNADYSDVMLT |
| | SEEEIKTTNPVATEEYGIVADNLQQQNTAPQIGTVNSQGVFPGSVWQDRDIYLQGQIWAKI |
| | PHTDGHFHPSPLMGGFGLKNPPPQILIKNTPVPADPPTEFNANKISSFITQYSTGQVTVEM |
| | EWELQKETSKRWNPEIQYTSNYYKSTSVDFAVNTEGVYKEFRSIGTRYLTRPL |
| 106 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISSQSGASNDNHYFGFSTPWGYFDFNRFHCHFSPRDWQRLINNNWGMRPKAM |
| | RVKIFNIQVKEVTQNDGTTTVANNLTSTVQIFADSSYELPYVMDAGQEGSLPPFPNDVFMV |
| | PQYGYLTLNNGSQAVGRSSFYCLEYFPSQMLRTGNNFEITYSFEKVPFHSMYAHSQSLDRL |
| | MNPLIDQYLYYLSRTNTPSGTTTQSRLQFSQAGASDIRDQSRNWLPGPSIRQQRVSKTSAD |
| | NNNSEYSWTGATKYHLNGRWSALTPGPPMATHKDDEEKFFPQSGVLIFGKQGSEKTNVDIE |
| | KLIFTSEEELAATNATDTDMWGSVSTNLQRGNRQAATADVNTQGAVPGMVWQNRDIYYQGP |
| | IWAKIPHTDGHFHPSPLIGGFGLKHPPPQIFIKNTPVPANPATTFSSTPVNSFITQYSTGQ |
| | VSVQIDWEIQKERSKRWNPEVQFTSNYNKSVNVDFTVDTNGVYTEPRAIGTRYLTHHL |
| 107 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKRLGESLQSNTYNGFSTPWGYFDFNRFHCHFSPRDWQRLINNNWGMRPKAMRVK |
| | IFNIQVKEVTTSNGETTVANNLTSTVQIFADSSYELPYVMDAGQEGSLPPFPNDVFMVPQY |
| | GYCGLVTGNTSQQQTDRNAFYCLEYFPSQMLRTGNNFEITYSFEKVPFHSMYAHSQSLDRL |
| | MNPLIDQYLYYLSRTNTPSGTTTQSRLQFSQAGASDIRDQSRNWLPGPCYRQQRVSKTSAD |
| | NNNSEYSWTGATKYHLNGRDSLVNPGPAMASHKDDEEKFFPQSGVLIFGKQGSEKTNVDIE |
| | KVMITDEEEIRTTNPVATEQYGSVSTNLQRGNRQAATADVNTQGAVPGMVWQNRDIYYQGP |
| | IWAKIPHTDGHFHPSPLIGGFGLKHPPPQIFIKNTPVPANPATTFSSTPVNSFITQYSTGQ |
| | VSVQIDWEIQKERSKRWNPEVQFTSNYGQQNSLLWAPDAAGKYTEPRAIGTRYLTHHL |
| 108 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISSQSGASNDNHYFGFSTPWGYFDFNRFHCHFSPRDWQRLINNNWGMRPKAM |
| | RVKIFNIQVKEVTQNDGTTTVANNLTSTVQIFADSSYELPYVMDAGQEGSLPPFPNDVFMV |
| | PQYGYLTLNNGSQAVGRSSFYCLEYFPSQMLRTGNNFEITYSFEKVPFHSMYAHSQSLDRL |
| | MNPLIDQYLYYLSRTNTPSGTTTQSRLQFSQAGASDIRDQSRNWLPGPCYRQQRVSKTSAD |
| | NNNSEYSWTGATKYHLNGRDSLVNPGPAMASHKDDEEKFFPQSGVLIFGKQGSEKTNVDIE |
| | KVMITDEEEIRTTNPVATEQYGSVSTNLQRGNRQAATADVNTQGAVPGMVWQNRDIYYQGP |
| | IWAKIPHTDGHFHPSPLIGGFGLKHPPPQIFIKNTPVPANPATTFSSTPVNSFITQYSTGQ |
| | VSVQIDWEIQKERSKRWNPEVQFTSNYNKSVNVDFTVDTNGVYTEPRAIGTRYLTHHL |
| 109 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISNGTSGGATNDNTYFGFSTPWGYFDFNRFHCHFSPRDWQRLINNNWGMRPK |
| | AMRVKIFNIQVKEVTQNEGTKTVANNLTSTVQIFADSSYELPYVMDAGQEGSLPPFPNDVF |
| | MVPQYGYLTLNNGSQAVGRSSFYCLEYFPSQMLRTGNNFEITYSFEKVPFHSMYAHSQSLD |
| | RLMNPLIDQYLYYLSRTQTTGGTANTQTLGFSQGGPNTMANQAKNWLPGPSIRQQRVSTTT |
| | GQNNNSNFAWTAGTKYHLNGRWSALTPGPPMATHKDDEERFFPSNGILIFGKQNAARDNAD |
| | YSDLIFTSEEELAATNATDTDMWGIVADNLQQQNTAPQIGTVNSQGAVPGMVWQNRDIYYQ |
| | GPIWAKIPHTDGHFHPSPLIGGFGLKHPPPQIFIKNTPVPANPATTFSSTPVNSFITQYST |
| | GQVSVQIDWEIQKERSKRWNPEVQFTSNYYKSTSVDFAVNTEGVYTEPRAIGTRYLTHHL |
| 110 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKRLGESLQSNTYNGFSTPWGYFDFNRFHCHFSPRDWQRLINNNWGMRPKAMRVK |
| | IFNIQVKEVTTSNGETTVANNLTSTVQIFADSSYELPYVMDAGQEGSLPPFPNDVFMVPQY |
| | GYCGLVTGNTSQQQTDRNAFYCLEYFPSQMLRTGNNFEITYSFEKVPFHSMYAHSQSLDRL |
| | MNPLIDQYLYYLSRTQTTGGTANTQTLGFSQGGPNTMANQAKNWLPGPCYRQQRVSTTTGQ |
| | NNNSNFAWTAGTKYHLNGRNSLANPGIAMATHKDDEERFFPSNGILIFGKQNAARDNADYS |
| | DVMLTSEEEIKTTNPVATEEYGIVADNLQQQNTAPQIGTVNSQGAVPGMVWQNRDIYYQGP |
| | IWAKIPHTDGHFHPSPLIGGFGLKHPPPQIFIKNTPVPANPATTFSSTPVNSFITQYSTGQ |
| | VSVQIDWEIQKERSKRWNPEVQFTSNYGQQNSLLWAPDAAGKYTEPRAIGTRYLTHHL |
| 111 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISNGTSGGATNDNTYFGFSTPWGYFDFNRFHCHFSPRDWQRLINNNWGMRPK |
| | AMRVKIFNIQVKEVTQNEGTKTVANNLTSTVQIFADSSYELPYVMDAGQEGSLPPFPNDVF |
| | MVPQYGYLTLNNGSQAVGRSSFYCLEYFPSQMLRTGNNFEITYSFEKVPFHSMYAHSQSLD |
| | RLMNPLIDQYLYYLSRTQTTGGTANTQTLGFSQGGPNTMANQAKNWLPGPCYRQQRVSTTT |
| | GQNNNSNFAWTAGTKYHLNGRNSLANPGIAMATHKDDEERFFPSNGILIFGKQNAARDNAD |
| | YSDVMLTSEEEIKTTNPVATEEYGIVADNLQQQNTAPQIGTVNSQGAVPGMVWQNRDIYYQ |
| | GPIWAKIPHTDGHFHPSPLIGGFGLKHPPPQIFIKNTPVPANPATTFSSTPVNSFITQYST |
| | GQVSVQIDWEIQKERSKRWNPEVQFTSNYYKSTSVDFAVNTEGVYTEPRAIGTRYLTHHL |
| 112 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISNSTSGGSSNDNAYFGFSTPWGYFDFNRFHCHFSPRDWQRLINNNWGMRPK |
| | AMRVKIFNIQVKEVTDNNGVKTVANNLTSTVQIFADSSYELPYVMDAGQEGSLPPFPNDVF |
| | MVPQYGYLTLNDGSQAVGRSSFYCLEYFPSQMLRTGNNFEITYSFEKVPFHSMYAHSQSLD |
| | RLMNPLIDQYLYYLSKTINGSGQNQQTLKFSVAGPSNMAVQGRNWLPGPSIRQQRVSTTVT |
| | QNNNSEFAWPGASSWALNGRWSALTPGPPMATHKEGEDRFFPLSGSLIFGKQGTGRDNVDA |
| | DKLIFTSEEELAATNATDTDMWGQVATNHQSAQAQAQTGWVQNQGAVPGMVWQNRDIYYQG |
| | PIWAKIPHTDGHFHPSPLIGGFGLKHPPPQIFIKNTPVPANPATTFSSTPVNSFITQYSTG |
| | QVSVQIDWEIQKERSKRWNPEVQFTSNYYKSTSVDFAVNTEGVYTEPRAIGTRYLTHHL |
| 113 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKRLGESLQSNTYNGFSTPWGYFDFNRFHCHFSPRDWQRLINNNWGMRPKAMRVK |
| | IFNIQVKEVTTSNGETTVANNLTSTVQIFADSSYELPYVMDAGQEGSLPPFPNDVFMVPQY |
| | GYCGLVTGNTSQQQTDRNAFYCLEYFPSQMLRTGNNFEITYSFEKVPFHSMYAHSQSLDRL |
| | MNPLIDQYLYYLSKTINGSGQNQQTLKFSVAGPSNMAVQGRNYIPGPSYRQQRVSTTVTQN |
| | NNSEFAWPGASSWALNGRNSLMNPGPAMASHKEGEDRFFPLSGSLIFGKQGTGRDNVDADK |
| | VMITNEEEIKTTNPVATESYGQVATNHQSAQAQAQTGWVQNQGAVPGMVWQNRDIYYQGPI |
| | WAKIPHTDGHFHPSPLIGGFGLKHPPPQIFIKNTPVPANPATTFSSTPVNSFITQYSTGQV |
| | SVQIDWEIQKERSKRWNPEVQFTSNYGQQNSLLWAPDAAGKYTEPRAIGTRYLTHHL |
| 114 | MTDGYLPDWLEDNLSEGVREWWALQPGAPKPKANQQHQDNARGLVLPGYKYLGPGNGLDKG |
| | EPVNAADAAALEHDKAYDQQLKAGDNPYLKYNHADAEFQQRLQGDTSFGGNLGRAVFQAKK |
| | RVLEPLGLVEQAGETAPGKKRPLIESPQQPDSSTGIGKKGKQPAKKKLVFEDETGAGDGPP |
| | EGSTSGAMSDDSEMRAAAGGAAVEGGQGADGVGNASGDWHCDSTWSEGHVTTTSTRTWVLP |
| | TYNNHLYKQISNSTSGGSSNDNAYFGFSTPWGYFDFNRFHCHFSPRDWQRLINNNWGMRPK |
| | AMRVKIFNIQVKEVTDNNGVKTVANNLTSTVQIFADSSYELPYVMDAGQEGSLPPFPNDVF |
| | MVPQYGYLTLNDGSQAVGRSSFYCLEYFPSQMLRTGNNFEITYSFEKVPFHSMYAHSQSLD |
| | RLMNPLIDQYLYYLSKTINGSGQNQQTLKFSVAGPSNMAVQGRNYIPGPSYRQQRVSTTVT |
| | QNNNSEFAWPGASSWALNGRNSLMNPGPAMASHKEGEDRFFPLSGSLIFGKQGTGRDNVDA |
| | DKVMITNEEEIKTTNPVATESYGQVATNHQSAQAQAQTGWVQNQGAVPGMVWQNRDIYYQG |
| | PIWAKIPHTDGHFHPSPLIGGFGLKHPPPQIFIKNTPVPANPATTFSSTPVNSFITQYSTG |
| | QVSVQIDWEIQKERSKRWNPEVQFTSNYYKSNNVEFAVNTEGVYTEPRAIGTRYLTHHL |
| 115 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLQISNSTSGGSSNDNAYFGFSTPWGYFDFNRFHCHFSPRDWQRLINNHWGLR |
| | PKSMQVRIFNIQVKEVTDNNGVKSNNLTSTVQIFADSTYELPYVMDAGQEGSLPPFPNDVF |
| | MVPQYGYLTLNDGSQAVGRSSFYCLEYFPSQMLRTGNNFEMVYKFENVPFHSMYAHSQSLD |
| | RLMNPLLDQYLYYLSKTINGSGQNQQTLKFSVAGPSNMAVQGRNWLPGPMMRQQRVSTTVT |
| | QNNNSEFAWPGASSWALNGRWSNFAPGTAMATHKEGEDRFFPLSGSLIFGKQGTGRDNVDA |
| | DKLMFTSEDELRATNPRDTDLFGQVATNHQSAQAQAQTGWVQNQGVYPGMVWQDRDIYYQG |
| | PIWAKIPHTDGHFHPSPLIGGFGLKSPPPQIFIKNTPVPANPATTFSPARINSFITQYSTG |
| | QVAVKIEWEIQKERSKRWNPEVQFTSNYYKSNNVEFAVNTEGVYKEPRAIGSRYLTNHL |
| 116 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLRLGSSNASDTFNGFSTPWGYFDFNRFHCHFSPRDWQRLINNHWGLRPKSMQ |
| | VRIFNIQVKEVTTSNGETTVSNNLTSTVQIFADSTYELPYVMDAGQEGSLPPFPNDVFMVP |
| | QYGYCGLVTGGSSQNQTDRNAFYCLEYFPSQMLRTGNNFEMVYKFENVPFHSMYAHSQSLD |
| | RLMNPLLDQYLYYLSKTINGSGQNQQTLKFSVAGPSNMAVQGRNYIPGPSYRQQRVSTTVT |
| | QNNNSEFAWPGASSWALNGRNSLMNPGPAMASHKEGEDRFFPLSGSLIFGKQGTGRDNVDA |
| | DKVMITNEEEIKTTNPVATESYGQVATNHQSAQAQAQTGWVQNQGVYPGMVWQDRDIYYQG |
| | PIWAKIPHTDGHFHPSPLIGGFGLKSPPPQIFIKNTPVPANPATTFSPARINSFITQYSTG |
| | QVAVKIEWEIQKERSKRWNPEVQFTSNYGAQDSLLWAPDNAGAYKEPRAIGSRYLTNHL |
| 117 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLQISNSTSGGSSNDNAYFGFSTPWGYFDFNRFHCHFSPRDWQRLINNHWGLR |
| | PKSMQVRIFNIQVKEVTDNNGVKSNNLTSTVQIFADSTYELPYVMDAGQEGSLPPFPNDVF |
| | MVPQYGYLTLNDGSQAVGRSSFYCLEYFPSQMLRTGNNFEMVYKFENVPFHSMYAHSQSLD |
| | RLMNPLLDQYLYYLSKTINGSGQNQQTLKFSVAGPSNMAVQGRNYIPGPSYRQQRVSTTVT |
| | QNNNSEFAWPGASSWALNGRNSLMNPGPAMASHKEGEDRFFPLSGSLIFGKQGTGRDNVDA |
| | DKVMITNEEEIKTTNPVATESYGQVATNHQSAQAQAQTGWVQNQGVYPGMVWQDRDIYYQG |
| | PIWAKIPHTDGHFHPSPLIGGFGLKSPPPQIFIKNTPVPANPATTFSPARINSFITQYSTG |
| | QVAVKIEWEIQKERSKRWNPEVQFTSNYYKSNNVEFAVNTEGVYKEPRAIGSRYLTNHL |
| 118 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLQISNGTSGGATNDNTYFGFSTPWGYFDFNRFHCHFSPRDWQRLINNHWGLR |
| | PKSMQVRIFNIQVKEVTQNEGTKSNNLTSTVQIFADSTYELPYVMDAGQEGSLPPFPNDVF |
| | MVPQYGYLTLNNGSQAVGRSSFYCLEYFPSQMLRTGNNFEMVYKFENVPFHSMYAHSQSLD |
| | RLMNPLLDQYLYYLSRTQTTGGTANTQTLGFSQGGPNTMANQAKNWLPGPMMRQQRVSTTT |
| | GQNNNSNFAWTAGTKYHLNGRWSNFAPGTAMATHKDDEERFFPSNGILIFGKQNAARDNAD |
| | YSDLMFTSEDELRATNPRDTDLFGIVADNLQQQNTAPQIGTVNSQGVYPGMVWQDRDIYYQ |
| | GPIWAKIPHTDGHFHPSPLIGGFGLKSPPPQIFIKNTPVPANPATTFSPARINSFITQYST |
| | GQVAVKIEWEIQKERSKRWNPEVQFTSNYYKSTSVDFAVNTEGVYKEPRAIGSRYLTNHL |
| 119 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLRLGSSNASDTFNGFSTPWGYFDFNRFHCHFSPRDWQRLINNHWGLRPKSMQ |
| | VRIFNIQVKEVTTSNGETTVSNNLTSTVQIFADSTYELPYVMDAGQEGSLPPFPNDVFMVP |
| | QYGYCGLVTGGSSQNQTDRNAFYCLEYFPSQMLRTGNNFEMVYKFENVPFHSMYAHSQSLD |
| | RLMNPLLDQYLYYLSRTQTTGGTANTQTLGFSQGGPNTMANQAKNWLPGPCYRQQRVSTTT |
| | GQNNNSNFAWTAGTKYHLNGRNSLANPGIAMATHKDDEERFFPSNGILIFGKQNAARDNAD |
| | YSDVMLTSEEEIKTTNPVATEEYGIVADNLQQQNTAPQIGTVNSQGVYPGMVWQDRDIYYQ |
| | GPIWAKIPHTDGHFHPSPLIGGFGLKSPPPQIFIKNTPVPANPATTFSPARINSFITQYST |
| | GQVAVKIEWEIQKERSKRWNPEVQFTSNYGAQDSLLWAPDNAGAYKEPRAIGSRYLTNHL |
| 120 | MSFVDHPPDWLESIGDGFREFLGLEAGPPKPKANQQKQDNARGLVLPGYKYLGPGNGLDKG |
| | DPVNFADEVAREHDLSYQKQLEAGDNPYLKYNHADAEFQEKLASDTSFGGNLGKAVFQAKK |
| | RILEPLGLVETPDKTAPAAKKRPLEQSPQEPDSSSGVGKKGKQPARKRLNFDDEPGAGDGP |
| | PPEGPSSGAMSTETEMRAAAGGNGGDAGQGAEGVGNASGDWHCDSTWSESHVTTTSTRTWV |
| | LPTYNNHLYLQISNGTSGGATNDNTYFGFSTPWGYFDFNRFHCHFSPRDWQRLINNHWGLR |
| | PKSMQVRIFNIQVKEVTQNEGTKSNNLTSTVQIFADSTYELPYVMDAGQEGSLPPFPNDVF |
| | MVPQYGYLTLNNGSQAVGRSSFYCLEYFPSQMLRTGNNFEMVYKFENVPFHSMYAHSQSLD |
| | RLMNPLLDQYLYYLSRTQTTGGTANTQTLGFSQGGPNTMANQAKNWLPGPCYRQQRVSTTT |
| | GQNNNSNFAWTAGTKYHLNGRNSLANPGIAMATHKDDEERFFPSNGILIFGKQNAARDNAD |
| | YSDVMLTSEEEIKTTNPVATEEYGIVADNLQQQNTAPQIGTVNSQGVYPGMVWQDRDIYYQ |
| | GPIWAKIPHTDGHFHPSPLIGGFGLKSPPPQIFIKNTPVPANPATTFSPARINSFITQYST |
| | GQVAVKIEWEIQKERSKRWNPEVQFTSNYYKSTSVDFAVNTEGVYKEPRAIGSRYLTNHL |
| 121 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPSNMAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 122 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSKTINGSGQNQQTLKFSVAGPSNMAVQGRNYIPGPSYRQQ |
| | RVSTTVTQNNNSEFAWPGASSWALNGRNSLMNPGPAMASHKEGEDRFFPLSGSLIFGKQGT |
| | GRDNVDADKVMITNEEEIKTTNPVATESYGQVATNHQSAQAQAQTGWVQNQGILPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 123 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNSTSGGSSNDNTYFGYSTPWGYFDFNRFHCHFSPRDWQRLINNN |
| | WGFRPKRLNFKLFNIQVKEVTDNNGVKTIANNLTSTVQVFTDSEYQLPYVLGSAHQGCLPP |
| | FPADVFMIPQYGYLTLNDGSQAVGRSSFYCLEYFPSQMLRTGNNFQFSYTFEDVPFHSSYA |
| | HSQSLDRLMNPLIDQYLYFLSRTINGSGQNQQTLQFSQAGPSSMANQARNWVPGPCYRQQR |
| | VSTTVTQNNNSEFAWPGATKYHLNGRDSLMNPGVAMASHKEGEDRFFPLSGVLIFGKQGTG |
| | RDNVDADKVMITNEEEIKTTNPVATEEYGAVATNHQSAQTLAVPFKAQAQTGLVHNQGVLP |
| | GMVWQNRDVYLQGPIWAKIPHTDGNFHPSPLMGGFGLKHPPPQILIKNTPVPADPPTTFNQ |
| | AKLNSFITQYSTGQVSVEIEWELQKENSKRWNPEIQYTSNYYKSNNVEFAVNTEGVYSEPR |
| | PIGTRFLTRNL |
| 124 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNSTSGGSSNDNTYFGYSTPWGYFDFNRFHCHFSPRDWQRLINNN |
| | WGFRPKRLNFKLFNIQVKEVTDNNGVKTIANNLTSTVQVFTDSEYQLPYVLGSAHQGCLPP |
| | FPADVFMIPQYGYLTLNDGSQAVGRSSFYCLEYFPSQMLRTGNNFQFSYTFEDVPFHSSYA |
| | HSQSLDRLMNPLIDQYLYFLSRTINGSGQNQQTLQFSQAGPSSMANQARNWVPGPCYRQQR |
| | VSTTVTQNNNSEFAWPGATKYHLNGRDSLMNPGVAMASHKEGEDRFFPLSGVLIFGKQGTG |
| | RDNVDADKVMITNEEEIKTTNPVATEEYGAVATNHQSAQAQAQTGLVHNQGVLPGMVWQNR |
| | DVYLQGPIWAKIPHTDGNFHPSPLMGGFGLKHPPPQILIKNTPVPADPPTTFNQAKLNSFI |
| | TQYSTGQVSVEIEWELQKENSKRWNPEIQYTSNYYKSNNVEFAVNTEGVYSEPRPIGTRFL |
| | TRNL |
| 125 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGSTNDNTYFGYSTPWGYFDFNRFHCHFSPRDWQRLINNN |
| | WGFRPKRLNFKLFNIQVKEVTTNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTINGSGQNQQTLKFSVAGPSNMAVQGRNYIPGPSYRQQR |
| | VSTTVTQNNNSEFAWPGASSWALNGRNSLMNPGPAMASHKEGEDRFFPLSGSLIFGKQGTG |
| | RDNVDADKVMITNEEEIKTTNPVATESYGQVATNHQSAQTLAVPFKAQAQTGWVQNQGVLP |
| | GMVWQNRDVYLQGPIWAKIPHTDGNFHPSPLMGGFGLKHPPPQILIKNTPVPADPPTTFNQ |
| | AKLNSFITQYSTGQVSVEIEWELQKENSKRWNPEIQYTSNYYKSTNVDFAVNTEGVYSEPR |
| | PIGTRFLTRNL |
| 126 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGSTNDNTYFGYSTPWGYFDFNRFHCHFSPRDWQRLINNN |
| | WGFRPKRLNFKLFNIQVKEVTTNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTINGSGQNQQTLKFSVAGPSNMAVQGRNYIPGPSYRQQR |
| | VSTTVTQNNNSEFAWPGASSWALNGRNSLMNPGPAMASHKEGEDRFFPLSGSLIFGKQGTG |
| | RDNVDADKVMITNEEEIKTTNPVATESYGQVATNHQSAQAQAQTGWVQNQGVLPGMVWQNR |
| | DVYLQGPIWAKIPHTDGNFHPSPLMGGFGLKHPPPQILIKNTPVPADPPTTFNQAKLNSFI |
| | TQYSTGQVSVEIEWELQKENSKRWNPEIQYTSNYYKSTNVDFAVNTEGVYSEPRPIGTRFL |
| | TRNL |
| 127 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNSTSGGSSNDNTYFGYSTPWGYFDFNRFHCHFSPRDWQRLINNN |
| | WGFRPKRLNFKLFNIQVKEVTDNNGVKTIANNLTSTVQVFTDSEYQLPYVLGSAHQGCLPP |
| | FPADVFMIPQYGYLTLNDGSQAVGRSSFYCLEYFPSQMLRTGNNFQFSYTFEDVPFHSSYA |
| | HSQSLDRLMNPLIDQYLYFLSRTINGSGQNQQTLQFSVAGPSNMAVQARNWVPGPCYRQQR |
| | VSTTVTQNNNSEFAWPGATKYHLNGRDSLMNPGVAMASHKEGEDRFFPLSGVLIFGKQGTG |
| | RDNVDADKVMITNEEEIKTTNPVATEEYGAVATNHQSAQAQAQTGLVHNQGVLPGMVWQNR |
| | DVYLQGPIWAKIPHTDGNFHPSPLMGGFGLKHPPPQILIKNTPVPADPPTTFNQAKLNSFI |
| | TQYSTGQVSVEIEWELQKENSKRWNPEIQYTSNYYKSNNVEFAVNTEGVYSEPRPIGTRFL |
| | TRNL |
| 128 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGSTNDNTYFGYSTPWGYFDFNRFHCHFSPRDWQRLINNN |
| | WGFRPKRLNFKLFNIQVKEVTTNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTQTTGGTANTQTLGFSQGGPNTMANQAKNWLPGPCYRQQ |
| | RVSTTTGQNNNSNFAWTAGTKYHLNGRNSLANPGIAMATHKDDEERFFPSNGILIFGKQNA |
| | ARDNADYSDVMLTSEEEIKTTNPVATEEYGIVADNLQQQNTAPQIGTVNSQGVLPGMVWQN |
| | RDVYLQGPIWAKIPHTDGNFHPSPLMGGFGLKHPPPQILIKNTPVPADPPTTFNQAKLNSF |
| | ITQYSTGQVSVEIEWELQKENSKRWNPEIQYTSNYYKSTNVDFAVNTEGVYSEPRPIGTRF |
| | LTRNL |
| 129 | MAADGYLPDWLEDNLSEGIREWWALKPGAPQPKANQQHQDNARGLVLPGYKYLGPGNGLDK |
| | GEPVNAADAAALEHDKAYDQQLKAGDNPYLKYNHADAEFQERLKEDTSFGGNLGRAVFQAK |
| | KRLLEPLGLVEEAAKTAPGKKRPVEQSPQEPDSSAGIGKSGAQPAKKRLNFGQTGDTESVP |
| | DPQPIGEPPAAPSGVGSLTMASGGGAPVADNNEGADGVGSSSGNWHCDSQWLGDRVITTST |
| | RTWALPTYNNHLYKQISNGTSGGSTNDNAYFGYSTPWGYFDFNRFHCHFSPRDWQRLINNN |
| | WGFRPKRLNFKLFNIQVKEVTTNEGTKTIANNLTSTVQVFTDSDYQLPYVLGSAHEGCLPP |
| | FPADVFMIPQYGYLTLNNGSQAVGRSSFYCLEYFPSQMLRTGNNFQFSYEFENVPFHSSYA |
| | HSQSLDRLMNPLIDQYLYFLSRTQTTGTAGTQTLKFSVAGPSNMAVQGRNYIPGPSYRQQR |
| | VSTTTNQNNNSNFAWTGASSWALNGRNSLMNPGPAMASHKDDEDRFFPSSGSLIFGKQGAG |
| | NDNVDYSQVMITNEEEIKTTNPVATESYGQVATNNQSANTQAQTGWVQNQGILPGMVWQDR |
| | DVYLQGPIWAKIPHTDGNFHPSPLMGGFGMKHPPPQILIKNTPVPADPPTAFNKDKLNSFI |
| | TQYSTGQVSVEIEWELQKENSKRWNPEIQYTSNYYKSTNVDFAVNTEGVYSEPRPIGTRFL |
| | TRNL |
| 130 | MAADGYLPDWLEDNLSEGIREWWALKPGAPQPKANQQHQDNARGLVLPGYKYLGPGNGLDK |
| | GEPVNAADAAALEHDKAYDQQLKAGDNPYLKYNHADAEFQERLKEDTSFGGNLGRAVFQAK |
| | KRLLEPLGLVEEAAKTAPGKKRPVEQSPQEPDSSAGIGKSGAQPAKKRLNFGQTGDTESVP |
| | DPQPIGEPPAAPSGVGSLTMASGGGAPVADNNEGADGVGSSSGNWHCDSQWLGDRVITTST |
| | RTWALPTYNNHLYKQISNSTSGGSSNDNAYFGYSTPWGYFDFNRFHCHFSPRDWQRLINNN |
| | WGFRPKRLNFKLFNIQVKEVTDNNGVKTIANNLTSTVQVFTDSDYQLPYVLGSAHEGCLPP |
| | FPADVFMIPQYGYLTLNDGSQAVGRSSFYCLEYFPSQMLRTGNNFQFSYEFENVPFHSSYA |
| | HSQSLDRLMNPLIDQYLYFLSRTQTTGTAGTQTLQFSQAGPSSMANQARNWVPGPCYRQQR |
| | VSTTTNQNNNSNFAWTGATKYHLNGRDSLMNPGVAMASHKDDEDRFFPSSGVLIFGKQGAG |
| | NDNVDYSQVMITNEEEIKTTNPVATEEYGAVATNNQSANTQAQTGLVHNQGILPGMVWQDR |
| | DVYLQGPIWAKIPHTDGNFHPSPLMGGFGMKHPPPQILIKNTPVPADPPTAFNKDKLNSFI |
| | TQYSTGQVSVEIEWELQKENSKRWNPEIQYTSNYYKSNNVEFAVNTEGVYSEPRPIGTRFL |
| | TRNL |
| 131 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNGTSGGSTNDNTYFGYSTPWGYFDFNRFHCHFSPRDWQRLINN |
| | NWGFRPKRLSFKLFNIQVKEVTTNEGTKTIANNLTSTIQVFTDSEYQLPYVLGSAHQGCLP |
| | PFPADVFMIPQYGYLTLNNGSQAVGRSSFYCLEYFPSQMLRTGNNFQFTYTFEDVPFHSSY |
| | AHSQSLDRLMNPLIDQYLYFLSRTQTTGTAGTQTLGFSQGGPNTMANQAKNWLPGPCYRQQ |
| | RVSTTTNQNNNSNFAWTGGTKYHLNGRNSLANPGIAMATHKDDEDRFFPSSGILIFGKQGA |
| | GNDNVDYSQVMLTSEEEIKTTNPVATEEYGIVATNNQSANTQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSTNVDFAVNTEGVYSEPRPIGTRF |
| | LTRNL |
| 132 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNGTSGGATNDNTYFGYSTPWGYFDFNRFHCHFSPRDWQRLINN |
| | NWGFRPKRLSFKLFNIQVKEVTQNEGTKTIANNLTSTIQVFTDSEYQLPYVLGSAHQGCLP |
| | PFPADVFMIPQYGYLTLNNGSQAVGRSSFYCLEYFPSQMLRTGNNFQFTYTFEDVPFHSSY |
| | AHSQSLDRLMNPLIDQYLYFLSRTQTTGTAGTQTLQFSQAGPSSMANQARNWVPGPCYRQQ |
| | RVSTTTNQNNNSNFAWTGATKYHLNGRDSLMNPGVAMASHKDDEDRFFPSSGVLIFGKQGA |
| | GNDNVDYSQVMITNEEEIKTTNPVATEEYGAVATNNQSANTQAQTGLVHNQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSTSVDFAVNTEGVYSEPRPIGTRF |
| | LTRNL |
| 133 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGATNDNTYFGYSTPWGYFDFNRFHCHFSPRDWQRLINNN |
| | WGFRPKRLNFKLFNIQVKEVTQNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTQTTGGTANTQTLQFSQAGPSSMANQARNWVPGPCYRQQ |
| | RVSTTTGQNNNSNFAWTAATKYHLNGRDSLMNPGVAMASHKDDEERFFPSNGVLIFGKQNA |
| | ARDNADYSDVMITNEEEIKTTNPVATEEYGAVADNLQQQNTAPQIGLVHNQGVLPGMVWQN |
| | RDVYLQGPIWAKIPHTDGNFHPSPLMGGFGLKHPPPQILIKNTPVPADPPTTFNQAKLNSF |
| | ITQYSTGQVSVEIEWELQKENSKRWNPEIQYTSNYYKSTSVDFAVNTEGVYSEPRPIGTRF |
| | LTRNL |
| 134 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLSRTQNQSGSAQNKDLGFSQGGPNTMANQAKNWLPGPCYRQQ |
| | RVSKTKTDNNNSNFTWTGATKYHLNGRNSLANPGIAMASHKDDKDKFFPMSGILIFGKESA |
| | GASNTALDNVMLTSEEEIKTTNPVATEEYGIVAVNLQSSSTDPATGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 135 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLSRTQNQSGSAQNKDLGFSRGSPAGMSVQAKNWLPGPCYRQQ |
| | RVSKTKTDNNNSNFTWTGATKYHLNGRNSLANPGIAMASHKDDKDKFFPMSGILIFGKESA |
| | GASNTALDNVMLTSEEEIKTTNPVATEEYGIVAVNLQSSSTDPATGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 136 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLNRTQNQSGSAQNKDLLFSRGSPAGMSVQPKNWLPGPCYRQQ |
| | RVSKTKTDNNNSNFTWTGASKYNLNGRESIINPGTAMASHKDDKDKFFPMSGVMIFGKESA |
| | GASNTALDNVMITDEEEIKATNPVATERFGTVAVNLQSSSTDPATGDVHVMGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 137 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPANMAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 138 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPTNMAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 139 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPSNAAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 140 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPSNIAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 141 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGPSNKAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 142 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISNSTSGGSSNDNTYFGYSTPWGYFDFNRFHCHFSPRDWQRLINN |
| | NWGFRPKRLSFKLFNIQVKEVTDNNGVKTIANNLTSTIQVFTDSEYQLPYVLGSAHQGCLP |
| | PFPADVFMIPQYGYLTLNDGSQAVGRSSFYCLEYFPSQMLRTGNNFQFTYTFEDVPFHSSY |
| | AHSQSLDRLMNPLIDQYLYYLSRTINGSGQNQQTLGFSVAGASNMAVQGRNWLPGPCYRQQ |
| | RVSTTVTQNNNSEFAWPGGTKYHLNGRNSLANPGIAMATHKEGEDRFFPLSGILIFGKQGT |
| | GRDNVDADKVMLTSEEEIKTTNPVATEEYGIVATNHQSAQAQAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYYKSNNVEFAVNTEGVYSEPRPIGTRY |
| | LTRNL |
| 143 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNSTSGGSSNDNTYFGYSTPWGYFDFNRFHCHFSPRDWQRLINNN |
| | WGFRPKRLNFKLFNIQVKEVTDNNGVKTIANNLTSTVQVFTDSEYQLPYVLGSAHQGCLPP |
| | FPADVFMIPQYGYLTLNDGSQAVGRSSFYCLEYFPSQMLRTGNNFQFSYTFEDVPFHSSYA |
| | HSQSLDRLMNPLIDQYLYFLSRTINGSGQNQQTLQFSQAGPSSMANQARNWVPGPCYRQQR |
| | VSTTVTQNNNSEFAWPGATKYHLNGRDSLMNPGVAMASHKEGEDRFFPLSGVLIFGKQGTG |
| | RDNVDADKVMITNEEEIKTTNPVATEEYGAVATNHQRGNRQAQTGLVHNQGVLPGMVWQNR |
| | DVYLQGPIWAKIPHTDGNFHPSPLMGGFGLKHPPPQILIKNTPVPADPPTTFNQAKLNSFI |
| | TQYSTGQVSVEIEWELQKENSKRWNPEIQYTSNYYKSNNVEFAVNTEGVYSEPRPIGTRFL |
| | TRNL |
| 144 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQIKSGSVDGSNDNTYFGYSTPWGYFDFNRFHCHFSPRDWQRLINNN |
| | WGFRPKRLSFKLFNIQVKEVTVQDSTTTIANNLTSTIQVFTDSEYQLPYVLGSAHQGCLPP |
| | FPADVFMIPQYGYLTLNRDNTENPTERSSFYCLEYFPSQMLRTGNNFQFTYTFEDVPFHSS |
| | YAHSQSLDRLMNPLIDQYLYYLSRTNTGGVQFSQGGPNTMANQAKNWLPGPCYRQQGWNLG |
| | SGVNRASVSAFATGTKYHLNGRNSLANPGIAMATNNLQGSNTYALENTLIFNSQPANPGTT |
| | ATYLEGNMLTSEEEIKTTNPVATEEYGIVATNNQSSTTAPATGTVNSQGALPGMVWQNRDV |
| | YLQGPIWAKIPHTDGNFHPSPLMGGFGLKHPPPQILIKNTPVPADPPTTFNQSKLNSFITQ |
| | YSTGQVSVEIEWELQKENSKRWNPEIQYTSNYNDPQFVDFAVNTEGVYSEPRPIGTRYLTR |
| | NL |
| 145 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQIKSGSVDGSNDNTYFGYSTPWGYFDFNRFHCHFSPRDWQRLINNNW |
| | GFRPKRLNFKLFNIQVKEVTVQDSTTTIANNLTSTVQVFTDSEYQLPYVLGSAHQGCLPPF |
| | PADVFMIPQYGYLTLNRDNTENPTERSSFYCLEYFPSQMLRTGNNFQFSYTFEDVPFHSSY |
| | AHSQSLDRLMNPLIDQYLYFLSRTNTGGVQFSQAGPSSMANQARNWVPGPCYRQQGWNLGS |
| | GVNRASVSAFATATKYHLNGRDSLMNPGVAMASNNLQGSNTYALENTLIFNSQPANPGTTA |
| | TYLEGNMITNEEEIKTTNPVATEEYGAVATNNQSSTTAPATGLVHNQGVLPGMVWQNRDVY |
| | LQGPIWAKIPHTDGNFHPSPLMGGFGLKHPPPQILIKNTPVPADPPTTFNQAKLNSFITQY |
| | STGQVSVEIEWELQKENSKRWNPEIQYTSNYNDPQFVDFAVNTEGVYSEPRPIGTRFLTRN |
| | L |
| 146 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLSRTQNQSGSAQNKDLGFSQGGPNTMANQAKNWLPGPCYRQQ |
| | RVSKTKTDNNNSNFTWTGATKYHLNGRNSLANPGIAMASHKDDKDKFFPMSGILIFGKESA |
| | GASNTALDNVMLTSEEEIKTTNPVATEEYGIVAVNLQSSSTDPATGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 147 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLSRTQNQSGSAQNKDLGFSRGSPAGMSVQAKNWLPGPCYRQQ |
| | RVSKTKTDNNNSNFTWTGATKYHLNGRNSLANPGIAMASHKDDKDKFFPMSGILIFGKESA |
| | GASNTALDNVMLTSEEEIKTTNPVATEEYGIVAVNLQSSSTDPATGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 148 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSASTGASNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLNRTQNQSGSAQNKDLLFSRGSPAGMSVQPKNWLPGPCYRQQ |
| | RVSKTKTDNNNSNFTWTGASKYNLNGRESIINPGTAMASHKDDKDKFFPMSGVMIFGKESA |
| | GASNTALDNVMITDEEEIKATNPVATERFGTVAVNLQSSSTDPATGDVHVMGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNYAKSANVDFAVNTEGVYSEPRPIGTRY |
| | LTRNL |
| 149 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSETAGSTNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLSRTQSNPGGTAGNRELGFSQGGPSTMAEQAKNWLPGPCYRQ |
| | QRVSKTLDQNNNSNFAWTGATKYHLNGRNSLANPGIAMATHKDDEDRFFPSSGILIFGKTG |
| | ATNKTTLENVMLTSEEEIKTTNPVATEEYGIVSSNLQAANTAAQTGTVNSQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNFEKQTGVDFAVNTEGVYSEPRPIGTRY |
| | LTRNL |
| 150 | MAADGYLPDWLEDNLSEGIREWWALKPGAPKPKANQQKQDDGRGLVLPGYKYLGPFNGLDK |
| | GEPVNAADAAALEHDKAYDQQLQAGDNPYLRYNHADAEFQERLQEDTSFGGNLGRAVFQAK |
| | KRVLEPLGLVEEGAKTAPGKKRPVEPSPQRSPDSSTGIGKKGQQPARKRLNFGQTGDSESV |
| | PDPQPLGEPPAAPSGVGPNTMAAGGGAPMADNNEGADGVGSSSGNWHCDSTWLGDRVITTS |
| | TRTWALPTYNNHLYKQISSETAGSTNDNTYFGYSTPWGYFDFNRFHCHFSPRDWQRLINNN |
| | WGFRPKRLSFKLFNIQVKEVTTNDGVTTIANNLTSTIQVFTDSEYQLPYVLGSAHQGCLPP |
| | FPADVFMIPQYGYLTLNNGSQAVGRSSFYCLEYFPSQMLRTGNNFQFTYTFEDVPFHSSYA |
| | HSQSLDRLMNPLIDQYLYYLARTQSNPGGTAGNRELQFYQGGPSTMAEQAKNWLPGPCFRQ |
| | QRVSKTLDQNNNSNFAWTGATKYHLNGRNSLVNPGVAMATHKDDEDRFFPSSGVLIFGKTG |
| | ATNKTTLENVLMTNEEEIRPTNPVATEEYGIVSSNLQAANTAAQTQVVNNQGALPGMVWQN |
| | RDVYLQGPIWAKIPHTDGNFHPSPLMGGFGLKHPPPQILIKNTPVPADPPTTFNQSKLNSF |
| | ITQYSTGQVSVEIEWELQKENSKRWNPEIQYTSNFEKQTGVDFAVNTEGVYSEPRPIGTRY |
| | LTRNL |
| 151 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGATNDNTYFGYSTPWGYFDFNRFHCHFSPRDWQRLINNN |
| | WGFRPKRLNFKLFNIQVKEVTQNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTQTTGGTANTQTLQFSQAGPSSMANQARNWVPGPCYRQQ |
| | RVSTTTGQNNNSNFAWTAATKYHLNGRDSLMNPGVAMASHKDDEERFFPSNGVLIFGKQNA |
| | ARDNADYSDVMITNEEEIKTTNPVATEEYGAVADNLQQQNTAPQIGLVHNQGVLPGMVWQN |
| | RDVYLQGPIWAKIPHTDGNFHPSPLMGGFGLKHPPPQILIKNTPVPADPPTTFNQAKLNSF |
| | ITQYSTGQVSVEIEWELQKENSKRWNPEIQYTSNYYKSTSVDFAVNTEGVYSEPRPIGTRF |
| | LTRNL |
| 152 | MAADGYLPDWLEDNLSEGIREWWDLKPGAPKPKANQQKQDDGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPGKKRPVEQSPQEPDSSSGIGKTGQQPAKKRLNFGQTGDSESVP |
| | DPQPLGEPPAAPSGVGSNTMASGGGAPMADNNEGADGVGNSSGNWHCDSTWLGDRVITTST |
| | RTWALPTYNNHLYKQISNGTSGGATNDNTYFGYSTPWGYFDFNRFHCHFSPRDWQRLINNN |
| | WGFRPKRLNFKLFNIQVKEVTQNEGTKTIANNLTSTVQVFTDSEYQLPYVLGSAHQGCLPP |
| | FPADVFMIPQYGYLTLNNGSQAVGRSSFYCLEYFPSQMLRTGNNFQFSYTFEDVPFHSSYA |
| | HSQSLDRLMNPLIDQYLYFLSRTQTTGGTANTQTLQFSQGGPNTMANQARNWVPGPCYRQQ |
| | RVSTTTGQNNNSNFAWTAATKYHLNGRDSLMNPGVAMASHKDDEERFFPSNGVLIFGKQNA |
| | ARDNADYSDVMITNEEEIKTTNPVATEEYGAVADNLQQQNTAPQIGLVHNQGVLPGMVWQN |
| | RDVYLQGPIWAKIPHTDGNFHPSPLMGGFGLKHPPPQILIKNTPVPADPPTTFNQAKLNSF |
| | ITQYSTGQVSVEIEWELQKENSKRWNPEIQYTSNYYKSTSVDFAVNTEGVYSEPRPIGTRF |
| | LTRNL |
| 153 | MAADGYLPDWLEDNLSEGIREWWDLKPGAPKPKANQQKQDNGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPAKKRPVEPSPQRSPDSSTGIGKKGQQPARKRLNFGQTGDSESV |
| | PDPQPLGEPPAAPSSVGSGTVAAGGGAPMADNNEGADGVGNASGNWHCDSTWLGDRVITTS |
| | TRTWALPTYNNHLYKQISNGTSGGSTNDNTYFGYSTPWGYFDFNRFHCHFSPRDWQRLINN |
| | NWGFRPKKLRFKLFNIQVKEVTQNEGTKTIANNLTSTIQVFSDSEYQLPYVLGSAHQGCLP |
| | PFPADVFMIPQYGYLTLNNGSQSVGRSSFYCLEYFPSQMLRTGNNFEFSYSFEDVPFHSSY |
| | AHSQSLDRLMNPLIDQYLYYLARTQSTGGTAGTQQLQFYQGGPSTMAEQAKNWLPGPCFRQ |
| | QRVSTTLSQNNNSNFAWTGATKYHLNGRNSLVNPGVAMATHKDDEERFFPSSGVLIFGKQG |
| | AGKDNVDYSSVLMTNEEEIRPTNPVATEEYGIVADNLQQQNAAPIVQVVNNQGALPGMVWQ |
| | NRDVYLQGPIWAKIPHTDGNFHPSPLMGGFGLKHPPPQILIKNTPVPANPPEVFTPAKFAS |
| | FITQYSTGQVSVEIEWELQKENSKRWNPEIQYTSNYYKSTNVDFAVDSQGVYSEPRPIGTR |
| | YLTRNL |
| 154 | MAADGYLPDWLEDNLSEGIREWWDLKPGAPKPKANQQKQDNGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPAKKRPVEPSPQRSPDSSTGIGKKGQQPARKRLNFGQTGDSESV |
| | PDPQPLGEPPAAPSSVGSGTVAAGGGAPMADNNEGADGVGNASGNWHCDSTWLGDRVITTS |
| | TRTWALPTYNNHLYKQISNGTSGGSTNDNTYFGYSTPWGYFDFNRFHCHFSPRDWQRLINN |
| | NWGFRPKKLRFKLFNIQVKEVTQNEGTKTIANNLTSTIQVFSDSEYQLPYVLGSAHQGCLP |
| | PFPADVFMIPQYGYLTLNNGSQSVGRSSFYCLEYFPSQMLRTGNNFEFSYSFEDVPFHSSY |
| | AHSQSLDRLMNPLIDQYLYYLARTQSTGGTAGTQQLQFYQAGPNNMSAQAKNWLPGPCFRQ |
| | QRVSTTLSQNNNSNFAWTGATKYHLNGRNSLVNPGVAMATHKDDEERFFPSSGVLIFGKQG |
| | AGKDNVDYSSVLMTNEEEIRPTNPVATEEYGIVADNLQQQNAAPIVQVVNNQGALPGMVWQ |
| | NRDVYLQGPIWAKIPHTDGNFHPSPLMGGFGLKHPPPQILIKNTPVPANPPEVFTPAKFAS |
| | FITQYSTGQVSVEIEWELQKENSKRWNPEIQYTSNYYKSTNVDFAVDSQGVYSEPRPIGTR |
| | YLTRNL |
| 155 | MAADGYLPDWLEDNLSEGIREWWDLKPGAPKPKANQQKQDNGRGLVLPGYKYLGPFNGLDK |
| | GEPVNAADAAALEHDKAYDQQLKAGDNPYLRYNHADAEFQERLQEDTSFGGNLGRAVFQAK |
| | KRVLEPLGLVEEGAKTAPAKKRPVEPSPQRSPDSSTGIGKKGQQPARKRLNFGQTGDSESV |
| | PDPQPLGEPPAAPSSVGSGTVAAGGGAPMADNNEGADGVGNASGNWHCDSTWLGDRVITTS |
| | TRTWALPTYNNHLYKQISNGTSGGSTNDNTYFGYSTPWGYFDFNRFHCHFSPRDWQRLINN |
| | NWGFRPKKLRFKLFNIQVKEVTQNEGTKTIANNLTSTIQVFSDSEYQLPYVLGSAHQGCLP |
| | PFPADVFMIPQYGYLTLNNGSQSVGRSSFYCLEYFPSQMLRTGNNFEFSYSFEDVPFHSSY |
| | AHSQSLDRLMNPLIDQYLYYLSRTQSTGGTAGTQQLLFSQAGPNNMSAQAKNWLPGPCYRQ |
| | QRVSTTLSQNNNSNFAWTGATKYHLNGRDSLVNPGVAMATHKDDEERFFPSSGVLMFGKQG |
| | AGKDNVDYSSVMLTSEEEIKTTNPVATEQYGVVADNLQQQNAAPIVGAVNSQGALPGMVWQ |
| | NRDVYLQGPIWAKIPHTDGNFHPSPLMGGFGLKHPPPQILIKNTPVPANPPEVFTPAKFAS |
| | FITQYSTGQVSVEIEWELQKENSKRWNPEIQYTSNYYKSTNVDFAVDSQGVYSEPRPIGTR |
| | YLTRNL |
| 156 | MAADGYLPDWLEDNLSEGIREWWALKPGAPQPKANQQHQDNARGLVLPGYKYLGPGNGLDK |
| | GEPVNAADAAALEHDKAYDQQLKAGDNPYLKYNHADAEFQERLKEDTSFGGNLGRAVFQAK |
| | KRLLEPLGLVEEAAKTAPGKKRPVEQSPQEPDSSAGIGKSGAQPAKKRLNFGQTGDTESVP |
| | DPQPIGEPPAAPSGVGSLTMASGGGAPVADNNEGADGVGSSSGNWHCDSQWLGDRVITTST |
| | RTWALPTYNNHLYKQISNGTSGGSTNDNAYFGYSTPWGYFDFNRFHCHFSPRDWQRLINNN |
| | WGFRPKRLNFKLFNIQVKEVTQNEGTKTIANNLTSTVQVFTDSDYQLPYVLGSAHEGCLPP |
| | FPADVFMIPQYGYLTLNNGSQAVGRSSFYCLEYFPSQMLRTGNNFQFSYEFENVPFHSSYA |
| | HSQSLDRLMNPLIDQYLYYLSKTISTGGTAGTQQLKFSVAGPSNMAVQGRNYIPGPSYRQQ |
| | RVSTTLSQNNNSNFAWTGASSWALNGRNSLMNPGPAMATHKDDEERFFPSSGSLIFGKQGA |
| | GKDNVDYSSVMITNEEEIKTTNPVATESYGQVADNLQQQNAAPIVGWVQNQGILPGMVWQD |
| | RDVYLQGPIWAKIPHTDGNFHPSPLMGGFGMKHPPPQILIKNTPVPADPPTAFNKDKLNSF |
| | ITQYSTGQVSVEIEWELQKENSKRWNPEIQYTSNYYKSTNVDFAVNTEGVYSEPRPIGTRY |
| | LTRNL |
| 157 | MAADGYLPDWLEDNLSEGIREWWALKPGAPQPKANQQHQDNARGLVLPGYKYLGPGNGLDK |
| | GEPVNAADAAALEHDKAYDQQLKAGDNPYLKYNHADAEFQERLKEDTSFGGNLGRAVFQAK |
| | KRLLEPLGLVEEAAKTAPGKKRPVEQSPQEPDSSAGIGKSGAQPAKKRLNFGQTGDTESVP |
| | DPQPIGEPPAAPSGVGSLTMASGGGAPVADNNEGADGVGSSSGNWHCDSQWLGDRVITTST |
| | RTWALPTYNNHLYKQISNGTSGGSTNDNAYFGYSTPWGYFDFNRFHCHFSPRDWQRLINNN |
| | WGFRPKRLNFKLFNIQVKEVTQNEGTKTIANNLTSTVQVFTDSDYQLPYVLGSAHEGCLPP |
| | FPADVFMIPQYGYLTLNNGSQAVGRSSFYCLEYFPSQMLRTGNNFQFSYEFENVPFHSSYA |
| | HSQSLDRLMNPLIDQYLYYLSRTQSTGGTAGTQQLLFSQAGPNNMSAQAKNWLPGPCYRQQ |
| | RVSTTLSQNNNSNFAWTGATKYHLNGRDSLVNPGVAMATHKDDEERFFPSSGVLMFGKQGA |
| | GKDNVDYSSVMLTSEEEIKTTNPVATEQYGVVADNLQQQNAAPIVGAVNSQGALPGMVWQD |
| | RDVYLQGPIWAKIPHTDGNFHPSPLMGGFGMKHPPPQILIKNTPVPADPPTAFNKDKLNSF |
| | ITQYSTGQVSVEIEWELQKENSKRWNPEIQYTSNYYKSTNVDFAVNTEGVYSEPRPIGTRY |
| | LTRNL |

Numerous modifications and variations in the disclosure as set forth in the above illustrative examples are expected to occur to those skilled in the art. Consequently only such limitations as appear in the appended claims should be placed on the disclosure.

1. A method of treating a subject with multiple doses of a recombinant adeno-associated virus (rAAV) vector, the method comprising:
administering to a subject a first rAAV vector comprising a first capsid and a transgene encoding a therapeutic molecule, and
administering to the subject a second rAAV vector comprising a second capsid and a transgene encoding a therapeutic molecule,
wherein the transgene in the second rAAV vector encodes the same therapeutic molecule or a different therapeutic molecule as the transgene in the first rAAV vector.

2. A method of treating a disease or disorder in a subject in need thereof with multiple doses of a recombinant adeno-associated virus (rAAV) vector, the method comprising:
administering to a subject a first rAAV vector comprising a first capsid and a transgene encoding a therapeutic molecule useful for treating the disease or disorder, and
administering to the subject a second rAAV vector comprising a second capsid and a transgene encoding a therapeutic molecule useful for treating the disease or disorder,
wherein the transgene in the second capsid encodes the same therapeutic molecule or a different therapeutic molecule useful to treat the disease or disorder as the transgene in the first rAAV vector.

3. The method of claim 1, wherein the first and second capsids are phylogenetically distinct.

4. The method of claim 3, wherein the phylogenetic difference is based on a threshold level of sequence homology.

5. The method of claim 1, wherein the first and second capsids have amino acid sequence homology that is less than or equal to about 90%.

6. The method of claim 1, wherein the first and second capsids have less than or equal to about 85% homology in a VP1 capsid protein and/or less than or equal to about 85% homology in a VP3 capsid protein.

7. The method of claim 1, wherein the first capsid and/or second capsid exhibit low pre-existing immunity in the subject.

8. The method of claim 1, wherein the subject is human.

9. The method of claim 1, wherein the subject is human and is immunologically naïve to the first and/or second AAV vector.

10. The method of claim 9, wherein the subject has less than 1:2, 1:5, 1:10, 1:20, 1:50, 1:100, 1:200 or 1:300, 1:500, 1:1000 neutralizing antibody to the first or second AAV vector in serum.

11. The method of claim 10, wherein the neutralizing antibody levels are measured in a neutralizing antibody assay.

12. The method of claim 1, wherein the first capsid or second capsid is selected from the group consisting of AAV1, AAV2, AAV3, AAV4, AAV5, AAV6, AAV7, AAV8, AAV9, AAV10, AAV11, AAV12, AAV13, AAV3b, LK03, rh74.j, rh10, bovine, AAVGoat, Bba.41, Bba.47, Bba.49, Bba.33, Bba.45, Bba.46, Bba.50, Bba.51, RN35, Anc110_9VR, AAV_go.1, AAVs listed in Table 4, AAV listed in Table 5, and/or variants thereof.

13. (canceled)

14. (canceled)

15. The method of claim 1, wherein the first capsid and/or second capsid is an engineered or chimeric capsid.

16. The method of claim 1, wherein the first or second capsid comprises a chimeric capsid protein having a VP1 amino acid sequence of a recipient backbone AAV capsid comprising variable regions I, II, III, IV, V, VI, VII, VIII and IX, except wherein one or more of variable regions I, II, III, IV, V, VI, VII, VIII, or IX is replaced by the corresponding variable region from one or more donor AAV capsids.

17. (canceled)

18. The method of claim 16, wherein the VP1 amino acid sequence of the recipient backbone AAV capsid is any of SEQ ID NOS:1-89 or 158-164 and the corresponding variable region from one or more donor AAV capsids is from SEQ ID NOS:1-89 or 158-164, and the recipient backbone AAV capsid sequence and the donor AAV capsid sequences are different.

19. The method of claim 15, wherein the chimeric capsid comprises the amino acid sequence of any one of SEQ ID NOS:90-157.

20. The method of claim 1, wherein the first or second capsid comprises an amino acid sequence that is at least 95% identical to (i) any one of SEQ ID NOs:15-89 or 158-164, (ii) the VP2 region of any one of SEQ ID NOs: 15-89 or 158-164, or (iii) the VP3 region of any one of SEQ ID NOs: 15-89 or 158-164.

21. The method of claim 1, wherein the first or second capsid comprises the amino acid sequence of (i) any one of SEQ ID NOS:15-89, (ii) the VP2 region of any one of SEQ ID NOS:15-89, or (iii) the VP3 region of any one of SEQ ID NOS:15-89.

22. The method of claim 1, wherein the first or second rAAV vector comprises a nucleic acid sequence encoding an adeno-associated virus (AAV) capsid protein having an amino acid sequence that is at least 95% identical to (i) any one of SEQ ID NOS:15-89 or 158-164, (ii) the VP2 region of any one of SEQ ID NOS:15-89 or 158-164, or (iii) the VP3 region of any one of SEQ ID NOS:15-89 or 158-164.

23. The method of claim 22, wherein the nucleic acid sequence is operably linked to a heterologous regulatory element that controls expression of the transgene in a host cell.

24. The method of claim 23, wherein the host cell is a liver cell or muscle cell.

25. The method of claim 1, wherein the first capsid is selected from the group consisting of AA5, Bba.49, Bb47 and bovine and the second capsid is selected from the group consisting of AA5, Bba.49, Bb47 and bovine.

26. The method of claim 1, wherein the first capsid is selected from the group consisting of LK03, AA5, Bba.49 and bovine, and the second capsid is selected from the group consisting of LK03, AAV5, Bba.49 and bovine.

27. The method of claim 1, wherein the first capsid is selected from the group consisting of rh10, AA5, Bba.49 and bovine, and the second capsid is selected from the group consisting of rh10, AAV5, Bba.49 and bovine.

28. The method of claim 1, wherein the first capsid is selected from the group consisting of AAV8, AA5, Bba.49 and bovine, and the second capsid is selected from the group consisting of AAV8, AAV5, Bba.49 and bovine.

29. The method of claim 1, wherein the first capsid or second capsid is AAV9 or Bba.41.

30. The method of claim 1, wherein the transgene expresses a heterologous protein that is maintained in the subject at a therapeutically effective level.

31. The method of claim 30, wherein the heterologous protein is selected from the group consisting of Factor VIII, Factor IX, ATP7B protein, C1 esterase inhibitor (C1-INH), alpha 1 antitrypsin, and galactose-1-phosphate uridyl transferase (GALT), dystrophin, a mini-dystrophin, microdystrophin, phenylalanine hydroxylase (PAH), alpha-galactosidase A, and glucocerebrosidase.

32. The method of claim 31, wherein expression of the heterologous protein is sufficient to treat a disorder or disease selected from the group consisting of hemophilia A, hemophilia B, Wilson's disease, hereditary angioedema (HAE), alpha 1 antitrypsin deficiency, galactosemia, Duchenne's Muscular Dystrophy or other muscular dystrophies, phenylketonuria (PKU), Fabry Disease, and Gaucher Disease.
